(19) United States
(12) Patent Application Publication
(10) Pub. No.: US 2012/0258975 A1
(43) Pub. Date: Oct. 11, 2012

(54) Potent Small Molecule Inhibitors of Autophagy, and Methods of Use Thereof

(75) Inventors: Junying Yuan, Newton, MA (US); Dawei Ma, Shanghai (CN); Junli Liu, Shanghai (CN); Lihong Zhang, Shanghai (CN)

(73) Assignees: Shanghai Institute of Organic Chemistry, Shanghai (CN); President and Fellows of Harvard College, Cambridge, MA (US)

(21) Appl. No.: 13/382,572

(22) PCT Filed: Jul. 21, 2010
(86) PCT No.: PCT/US2010/042759

§ 371 Date: Jun. 19, 2012

Related U.S. Application Data
(60) Provisional application No. 61/227,164, filed on Jul. 21, 2009.
(60) Provisional application No. 61/296,735, filed on Jan. 20, 2010.

(51) Int. Cl. A61K 31/517; A61P 35/00; A61P 29/00; A61P 31/00; C07D 471/04; A61P 1/18; A61P 31/04; A61P 31/12; C12N 9/99; A61K 31/519; C07D 239/94; A61P 25/28

(52) U.S. Cl. 514/264.11; 544/293; 514/266.4; 435/184; 514/266.24; 544/279

(57) ABSTRACT

Certain aspects of the invention relates to small molecule autophagy inhibitors of the formula (I), and their use for treatment and prevention of cancers and acute pancreatitis. As disclosed herein, a small molecule inhibitor of autophagy was been identified from an image-based screen in a known bioactive library. It was found that this autophagy inhibitor functions by promoting the degradation of type III PI3 kinase complex which is required for initiating autophagy. Medicinal chemistry studies led to small molecular autophagy inhibitors with improved potency and selectivity. (I)

### RELATED APPLICATIONS

This application claims the benefit of priority to U.S. Provisional Patent Application Ser. No. 61/296,735, filed Jan. 20, 2010 and U.S. Provisional Patent Application Ser. No. 61/227,164, filed Jul. 21, 2009; the contents of both of which are hereby incorporated by reference in their entireties.

### GOVERNMENT SUPPORT

This invention was made with government support under PO1 AG027916, R37 AG012859 and DP1 OD000580 awarded by the National Institutes of Health. The government has certain rights in the invention.

### BACKGROUND

Vps34 (vacuolar protein sorting 34), a type III PtdIns3 kinase (phosphatidylinositol 3-kinase), was first identified as a regulator of vacuolar hydrolase sorting in yeast (Herman and Emr, 1990). Vps34 specifically phosphorylates the D-3 position on the inositol ring of phosphatidylinositol (PtdIns) to produce PtdIns3P (Schu, P. V., Takegawa, K., Fry, M. J., Stack, J. H., Waterfield, M. D., and Emr, S. D. (1993) Phosphatidylinositol 3-kinase encoded by yeast VPS34 gene essential for protein sorting. Science 260, 88-91). PtdIns3P has been implicated in the control of multiple key intracellular membrane trafficking pathways, including endosome to lysosome transport, retrograde endosome to Golgi traffic, multivesicular body formation and autophagy (Herman, P. K., and Emr, S. D. (1990). Characterization of VPS34, a gene required for vacuolar protein sorting and vacuole segregation in Saccharomyces cerevisiae. Mol Cell Biol 10, 6742-6754; Kihara, A., Noda, T., Ishihara, N., and Ohsumi, Y. (2001). Two distinct Vps34 phosphatidylinositol 3-kinase complexes function in autophagy and carboxypeptidase Y sorting in Saccharomyces cerevisiae. J Cell Biol 152, 519-530). PtdIns3P is required for the initiation of autophagy, an evolutionarily conserved catabolic mechanism involved in the turnover of intracellular organelles and large protein complexes.

Vps34 is present in two complexes in yeast: complex I (Vps34, Vps15, Vps30/Atg6, and Atg14) involved in autophagy, and complex II (Vps34, Vps15, Vps30/Atg6, and Vps38) in the vacuolar protein sorting pathway (Kihara et al., 2001, cited above). In mammalian cells, Vps34 is found in at least two protein complexes, Vps34 complex I and Vps34 complex II, that may function similarly to their homologous complexes in yeast. The two mammalian Vps34 complexes share the core components of Vps34, Beclin1 and p150, which are homologous to yeast Vps34, Vps30/Atg6 and Vps15, respectively. In addition, the complex I contains Atg14L, the mammalian orthologue of yeast Atg14, which localizes to the isolation membrane/phagophore during starvation and is essential for autophagosome formation; while the complex II contains UVRAG, a homologue of Vps38 in yeast, which primarily localizes to late endosomes (Itakura, E., Kishi, C., Inoue, K., and Mizushima, N. (2008). Beclin 1 forms two distinct phosphatidylinositol 3-kinase complexes with mammalian Atg14 and UVRAG. Mol Biol Cell 19, 5360-5372; Liang, C., Feng, P., Ku, B., Dotan, I., Canaani, D., Oh, B. H., and Jung, J. U. (2006). Autophagic and tumour suppressor activity of a novel Beclin1-binding protein UVRAG. Nat Cell Biol 8, 688-699; Matsunaga, K., Saitoh, T., Tabata, K., Omori, H., Satoh, T., Kurotori, N., Maejima, I., Shirahama-Noda, K., Ichimura, T., Isobe, T., et al. (2009). Two Beclin 1-binding proteins, Atg14L and Rubicon, reciprocally regulate autophagy at different stages. Nat Cell Biol 11, 385-396.; Zhong, Y., Wang, Q. J., Li, X., Yan, Y., Backer, J. M., Chait, B. T., Heintz, N., and Yue, Z. (2009). Distinct regulation of autophagic activity by Atg14L and Rubicon associated with Beclin 1-phosphatidylinositol-3-kinase complex. Nat Cell Biol 11(4), 468-476). Interestingly, the stabilities of different components of Vps34 complexes are co-dependent upon each other as knockdown of one component often reduces the levels of others in the complexes (Itakura et al., 2008, cited above). However, we still know every little about the mechanisms that regulate the stability of Vps34 complexes which may play an important role in regulating multiple vesicular trafficking pathways.

Autophagy is a catabolic process mediating the turnover of intracellular constituents in a lysosome-dependent manner (Levine, B., and Klionsky, D. J. (2004). Development by self-digestion: molecular mechanisms and biological functions of autophagy. Dev Cell 6, 463-477). Autophagy is initiated by the formation of an isolation membrane, which expands to engulf portion of cytoplasm, including large protein complexes and defective organelles, by forming a double membrane vesicle, termed autophagosome. The contents of an autophagosome are degraded by lysosomal hydrolases after its fusion with a lysosome to form an autolysosome. Autophagy has been studied extensively in unicellular eukaryotes as a strategy to survive starvation conditions, as products of autophagic degradation such as free amino acids, fatty acids and nucleotides, can be used by the cell as building blocks or a source of energy in order to help survive under nutrient limiting conditions (Levine, B., and Klionsky, D. J. (2004). Development by self-digestion: molecular mechanisms and biological functions of autophagy. Dev Cell 6, 463-477; and Levine, B., and Kroemer, G. (2008). Autophagy in the pathogenesis of disease. Cell 132, 27-42).

The core molecular machinery of autophagy is controlled by the protein products encoded by a group of ATG genes evolutionarily conserved from yeast to mammals. Nucleation of autophagic vesicles requires PtdIns3P, the product of type III PI3 kinase complex including Beclin 1 (mammalian homolog of yeast Atg6) and Vps34, as well as two ubiquitin-like molecules, Atg12 and LC3 (homolog of Atg8), which function sequentially in mediating the formation of autophagosomes. In the first ubiquitination-like reaction, Atg12 is conjugated to Atg5 and forms a large multimeric protein complex, which plays a key role in determining the nucleation of autophagosome. In the second reaction, LC3 is conjugated to phosphatidyl-ethanolamine, resulting in membrane translocation important for the elongation and closure of autophagosome (Fujita, N., Itoh, T., Omori, H., Fukuda, M., Noda, T., and Yoshimori, T. (2008). The Atg16L Complex Specifies the Site of LC3 Lipidation for Membrane Biogenesis in Autophagy. Mol Biol Cell 19, 2092-2100; and Levine, B., and Kroemer, G. (2008). Autophagy in the pathogenesis of disease. Cell 132, 27-42).

In metazoans, autophagy functions as an essential intracellular catabolic mechanism involved in cellular homeostasis by mediating the turnover of malfunctioning, aged or damaged proteins and organelles (Levine, B., and Kroemer, G. (2008). Autophagy in the pathogenesis of disease. Cell 132, 27-42). Down-regulation of autophagy contributes to neurodegeneration by increasing the accumulation of misfolded proteins (Hara, T., Nakamura, K., Matsui, M., Yamamoto, A., Nakahara, Y., Suzuki-Migishima, R., Yokoyama, M., Mishima, K., Saito, I., Okano, H., et al. (2006). Suppression of basal autophagy in neural cells causes neurodegenerative disease in mice. Nature 441, 885-889; and Komatsu, M., Waguri, S., Chiba, T., Murata, S., Iwata, J., Tanida, I., Ueno, T., Koike, M., Uchiyama, Y., Kominami, E., et al. (2006). Loss of autophagy in the central nervous system causes neurodegeneration in mice. Nature 441, 880-884). Autophagy can also be activated in response to many forms of cellular stress beyond nutrient starvation, including DNA damage, ER stress and invasion by intracellular pathogens, and has been shown to participate in both innate and acquired immunity (Schmid, D., Dengjel, J., Schoor, O., Stevanovic, S., and Munz, C. (2006). Autophagy in innate and adaptive immunity against intracellular pathogens. J Mol Med 84, 194-202) as well as in tumor suppression (Liang, X. H., Jackson, S., Seaman, M., Brown, K., Kempkes, B., Hibshoosh, H., and Levine, B. (1999). Induction of autophagy and inhibition of tumorigenesis by beclin 1. Nature 402, 672-676). Mechanisms that regulate autophagy in mammalian cells are just beginning to be explored.

Autophagy plays an important role in regulating cellular homeostasis and contributes to cell survival, growth, differentiation and host defense responses. Dysregulation of autophagy has been implicated in multiple human diseases including cancer, neurodegeneration, inflammatory diseases and infectious diseases. Most of the currently knowledge on autophagy were derived from elegant genetic studies in yeast which led to the identification of autophagy “Atg” genes . Recent studies have demonstrated the evolutionary conservation of the core autophagy genes from yeast to mammal; however, the mechanism and regulation of mammalian autophagy have shown significant increases in the complexity which we still know very little.

Autophagy has been proposed to play complex roles in development and treatment of cancers. Activation of autophagy may promote tumor cell survival under metabolic stress and function as a tumor suppression mechanism by preventing necrotic cell death and subsequent inflammation which favors tumor growth (White, E. (2008). Autophagic cell death unraveled: Pharmacological inhibition of apoptosis and autophagy enables necrosis. Autophagy 4, 399-401). On the other hand, inhibition of autophagy may lead to genome instability through unknown mechanisms which might explain the increased frequency of beclin 1 heterozygosity in multiple lines of cancers (Qu, X., Yu, J., Bhagat, G., Furuya, N., Hibshoosh, H., Troxel, A., Rosen, J., Eskelinen, E. L., Mizushima, N., Ohsumi, Y., et al. (2003). Promotion of tumorigenesis by heterozygous disruption of the beclin 1 autophagy gene. J Clin Invest 112, 1809-1820; and Yue, Z., Jin, S., Yang, C., Levine, A. J., and Heintz, N. (2003). Beclin 1, an autophagy gene essential for early embryonic development, is a haploinsufficient tumor suppressor. Proc Natl Acad Sci USA 100, 15077-15082) and decreased expression of autophagy-related proteins in malignant epithelial ovarian cancer (Shen, Y., Li, D. D., Wang, L. L., Deng, R., and Zhu, X. F. (2008). Decreased expression of autophagy-related proteins in malignant epithelial ovarian cancer. Autophagy 4, 1067-8). Thus, chronic suppression of autophagy may stimulate tumorigenesis.

The proposed role of autophagy in anticancer therapy is opposite to that during tumorigenesis. Once a tumor is formed, acute inhibition of autophagy might be beneficial for the therapeutic goal by promoting radiosensitization and chemosensitization (Amaravadi, R. K., and Thompson, C. B. (2007). The roles of therapy-induced autophagy and necrosis in cancer treatment. Clin Cancer Res 13, 7271-7279). In an animal model of cancer therapy, inhibition of therapy-induced autophagy either with shRNA against a key autophagy gene ATG5 or with anti-malarial drug chloroquine enhanced cell death and tumor regression of Myc-driven tumors in which either activated p53 or alkylating chemotherapy was used to drive tumor cell death (Amaravadi, R. K., Yu, D., Lum, J. J., Bui, T., Christophorou, M. A., Evan, G. I., Thomas-Tikhonenko, A., and Thompson, C. B. (2007). Autophagy inhibition enhances therapy-induced apoptosis in a Myc-induced model of lymphoma. J Clin Invest 117, 326-336). Chloroquine causes a dose-dependent accumulation of large autophagic vesicles and enhances alkylating therapy-induced cell death to a similar degree as knockdown of ATG5. In another example, resistance to TRAIL was found to be reversed by a common approach of targeting specific components of autophagic process, such as Beclin1 or Vps34, for inhibition (Hou, W., Han, J., Lu, C., Goldstein, L. A., and Rabinowich, H. (2008). Enhancement of tumor-TRAIL susceptibility by modulation of autophagy. Autophagy 4, 940-943). In the case of chronic myelogenous leukemia (CML), inhibition of autophagy by chloroquine markedly enhanced death of a CML cell line, K562, induced by imatinib. Furthermore, imatinib-resistant cell lines, BaF3/T315I and BaF3/E255K, can be induced to die by co-treatment with imatinib and chloroquine. Thus, inhibition of autophagy sensitizes tumor cells to imatinib-induced cell death. The block of autophagy has been proposed to be a new strategy for the treatment of CML (Mishima, Y., Terui, Y., Taniyama, A., Kuniyoshi, R., Takizawa, T., Kimura, S., Ozawa, K., and Hatake, K. (2008). Autophagy and autophagic cell death are next targets for elimination of the resistance to tyrosine kinase inhibitors. Cancer Sci 99, 2200-8). These studies suggest that autophagy can promote resistance to DNA-damaging therapy. Since chloroquine is a blocker of lysosomes, it will be interesting to see if specific inhibitors targeting different steps of autophagy process also have the same effect in enhancing the effect of chemotherapies in cell-based assays and animal models.

In addition, autophagy has also been shown to play an important role in mediating cellular damage induced by acute pancreatitis. Autodigestion of the pancreas by its own prematurely activated digestive proteases is thought to be an important event in the onset of acute pancreatitis. A conditional knockout mouse that lacks the autophagy-related (Atg) gene Atg5 in the pancreatic acinar cells has shown significantly reduced severity of acute pancreatitis induced by cerulein (Ohmuraya, M., and Yamamura, K. (2008). Autophagy and acute pancreatitis: a novel autophagy theory for trypsinogen activation. Autophagy 4, 1060-1062). Thus autophagy exerts a detrimental effect in pancreatic acinar cells by activation of trypsinogen to trypsin. Inhibitors of autophagy may provide important new therapeutics for acute pancreatitis.

Further, small molecule inhibitors are important tools in exploring the cellular mechanisms in mammalian cells. However, the only available small molecule inhibitor of autophagy is 3-methyladenine (3-MA), which has a working concentration of about 10 mM and is highly non-specific. Therefore, there is an urgent need to develop highly specific small molecule tools that can be used to facilitate the studies of autophagy in mammalian cells.

### SUMMARY

The invention relates to in part to compounds that are inhibitors of autophagy, compositions comprising such compounds, and methods of using such compounds and compositions.

One aspect of the invention relates to a compounds of formula I:

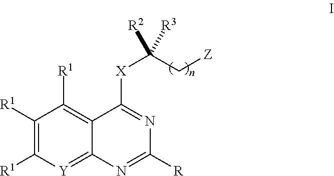

or a pharmaceutically acceptable salt, biologically active metabolite, solvate, hydrate, prodrug, enantiomer or stereoisomer thereof, wherein

n is 0, 1, 2, 3 or 4;

Y is —C(R¹)═ or —N═;

R is —H, lower alkyl, —NO₂, —OH, —NH₂, —NH(lower alkyl), —N(lower alkyl)₂, or lower alkynyl;

R¹ is independently selected for each occurrence from the group consisting of —H, —F, —Cl, —Br, —I, —NO₂, —OH, —NH₂, —NH(lower alkyl), —N(lower alkyl)₂, —CH₃, —CF₃, —C(═O)(lower alkyl), —CN, —O(lower alkyl), —O(lower fluoroalkyl), —S(═O)(lower alkyl), —S(═O)₂(lower alkyl) and —C(═O)O(lower alkyl);

R² and R³ are independently selected from the group consisting of —H, lower alkyl, lower fluoroalkyl, lower alkynyl and lower hydroxyalkyl;

X is —O—, —S—, —N(H)—, —N(lower alkyl)-, —CH₂—, —CH₂CH₂—, —CH₂CH₂CH₂—, —CH₂CH₂CH₂CH₂—, —CH₂CH₂CH₂CH₂CH₂— or —CH₂CH₂CH₂CH₂CH₂CH₂—; and

Z is phenyl, pyridyl, vinyl, morphinyl, phenanthrolinyl, naphthyl, furyl or benzo[d]thiazolyl; and optionally substituted with one or more substitutents selected from the group consisting of —CH₃, lower alkyl, fluoroalkyl, —OCH₃, —OCF₃, lower fluoroalkoxy, —F, —Cl, —Br, —I, —NO₂, lower alkyoxy, —NH(lower alkyl), —N(lower alkyl)₂, —CF₃, and 3,4-methylene dioxy.

Another aspect of the invention relates to a pharmaceutical composition comprising an compound of formula I, or a pharmaceutically acceptable salt, biologically active metabolite, solvate, hydrate, prodrug, enantiomer or stereoisomer thereof, and one or more pharmaceutically acceptable carriers, alone or in combination with another therapeutic agent. Such pharmaceutical compositions of the invention can be administered in accordance with a method of the invention, typically as part of a therapeutic regimen for treatment or prevention of conditions and disorders related to cancer or pancreatitis.

Another aspect of the invention relates to a method of treating or preventing cancer, pancreatitis or disease caused by an intracellular pathogen, comprising administering to a subject in need thereof a therapeutically effective amount of one or more compounds or pharmaceutical compositions of the invention.

### BRIEF DESCRIPTION OF THE FIGURES

FIG. 1 relates to identification of a small molecule inhibitor of autophagy by an image-based screen. A, structure of MBCQ. B, Quantitative analysis of LC3-GFP spot number per cell (a), spot size per cell (b), spot intensity per cell (c). The data are expressed as % of control vehicle treated cells. H4-LC3 cells were seeded in 96 well-plates and incubated with vehicle control (1% DMSO), 0.2 μM rapamycin with or without 10 μM MBCQ for indicated time, fixed with 4% paraformaldehyde and stained with 4,6-diamidino-2-phenylindole (DAPI, 3 μg/ml). Images of 1000 cells for each compound treatment were analyzed by ArrayScan HCS 4.0 Reader with a 20× objective (Cellomics, Pittsburgh, Pa.).

FIG. 2 depicts results relating to MBCQ inhibition of autophagy induced by starvation. Quantitative measurement of LC3-GFP spot number per cell (a), spot size per cell (b) and spot intensity per cell (c) using HCS and expressed as % of control. 3-MA (10 mM) or wortmannin (0.1 μM) were used as a positive control.

FIG. 3 depicts electron microscopy analysis of the effect of MBCQ on autophagy. H4 cells were treated with 0.1% DMSO (vehicle), rapamycin (0.2 μM), MBCQ (10 μM), or MBCQ and rapamycin for 4 h. The cells were processed and imaged by EM.

FIG. 4 depicts approaches to the generation of MBCQ derivatives.

FIG. 5 depicts results related to showing that active derivatives of MBCQ reduce the levels of LC3II in MEF cells. A, MEF cells were treated with DMSO (1‰), rapamycin (0.2 μM) alone, or together with MBCQ (10 μM), C43 (spautin) (10 μM) or C71 (10 μM), for 4 h. The cell lysates were collected for western blotting using anti-LC3 antibody. B, Electron microscopy confirmation of the autophagy inhibitory effects of C43 (spautin) on MEF cells. MEF cells were treated with vehicle control (1‰ DMSO), and other indicated compounds for 4 h. Rapamycin (0.2 μM) and C43 (spautin) (10 μM). Then the cells were fixed with glutaraldehyde and prepared the sample for EM assay. Bar, 1:11,000. Arrows indicate double and multi-membrane autophagosomic vesicles. N: nucleus.

FIG. 6 depicts results showing that MBCQ has little effect on H4 cell growth. A, H4 cells were treated with MBCQ (5 μM) for 5 days and harvested daily for cell number counting in the presence of trypan blue; B, H4 cells were treated with MBCQ (5 μM) for 24 h and 48 h, and then cells were fixed with 70% ethanol, stained with propidium iodide (40 μg/mL) and incubated with RNase (200 μg/mL solution for 30 min. The cell cycle profile and possible apoptotic cell death were analyzed by flow cytometer.

FIG. 7 depicts results showing that MBCQ and C43 (spautin) partially inhibit cell death of bax/bak DKO cells induced by etoposide. A-C, Bax/bak DKO cells were treated with MBCQ (10 μM), or 3-MA (10 mM) in the presence of or absent etoposide (8 μM) for 8 h or 24 h. A, Cell survival as demonstrated by images. B, cell survival as demonstrated by MTT assay. C, cells were collected for western blotting using anti-LC3 antibody. α-tubulin was used as a control. D-F, Bax/bak DKO cells were treated with spautin (10 μM) or indicated concentration, in the presence of or absent etoposide (8 μM) for 8 h or indicated time. D, Cell survival as demonstrated by images and E, MTT assay. F, Cells were collected for western blotting using anti-LC3 antibody. α-tubulin was used as a control.

FIG. 8 depicts results showing that MBCQ and C43 (spautin) reduce FYVE-RFP spots, but have no effect on the protein levels of FYVE-RFP. H4-FYVE cells were treated with DMSO (0.1%), MBCQ (10 μM) or C43 (spautin) (10 μM) for indicated time. A, The images were analyzed by fluorescence microscopy and quantified by HCS after fixing in 4% paraformaldehyde and stained with 4,6-diamidino-2-phenylindole (DAPI, 3 μg/mL). Images of 1000 cells for each compound treatment were analyzed by ArrayScan HCS 4.0 Reader with a 20× objective (Cellomics, Pittsburgh, Pa.). B, H4-FYVE cells were treated with DMSO (0.1%), RAPA (0.2 μM) alone, MBCQ (10 μM) or C43 (spautin) (10 μM) with or without RAPA (0.2 uM) for 8 h. The cell lysates were collected for western blotting using anti-RFP and anti-tubulin as a loading control.

FIG. 9 depicts results showing that MBCQ and C43 (spautin) selectively reduce the cellular levels of PtdIns3P. MEF cells were treated with DMSO (0.1%), RAPA (0.2 μM) alone, A, MBCQ (10 μM) or B, C43 (spautin) (10 μM) with or without RAPA (0.2 μM) for 3 h. The cellular PtdIns species were extracted and applied onto polyvinylidene fluoride membrane. The levels of PtdIns3P were detected using GST-PX domain protein and anti-GST antibody.

FIG. 10 depicts results showing that C43 (SPAYTIN) and its active derivatives selectively promote the degradation of Beclin1/Vps34/p150 complex. A, C43 (spautin) is not a direct inhibitor of Vps34 enzymatic activity. The exogenous HA-Vps34 complex immunoprecipitated using anti-HA from 293T was incubated with PtdIns in the presence of ³²P-ATP in the absence or presence of indicated concentrations of C43 (spautin) and wortmannin (10 uM) for 10 min at room temperature. The product was analyzed by thin layer chromatography and autoradiography. In lane 1, reaction buffer was used as negative control instead of Vps34/Beclin-1 complex. B, Treatment of MBCQ, C29 and C43 (spautin) reduced the levels of exogenous Vps34 and Beclin1. 293T cells were transfected with HA-Vps34 and flag-Beclin1 expression vectors. Twenty-four hours after the transfection, the cells were treated with indicated compounds for 12 h. The cell lysates were analyzed by western blotting using anti-HA, anti-flag or anti-tubulin. C, MBCQ and C43 (spautin) reduce the levels of GFP-P150 protein. 293T cells were transfected with GFP-P150 vector. Twenty-four h after the transfection, the cells were treated with MBCQ (10 μM), C43 (spautin) (10 μM) for an additional 4 h. The cell lysates were analyzed by western blotting using anti-GFP or anti-tubulin. D, MBCQ and C43 (spautin) reduce the levels of myc-Atg14 protein. 293T cells were transfected with myc-Atg14 vector. Twenty-four h after the transfection, the cells were treated with MBCQ (10 μM), C43 (spautin) (10 μM) for an additional 4 h. The cell lysates were analyzed by western blotting using anti-myc or anti-tubulin. E, H4 cells were treated with Rapamycin (0.2 μM) with or without C43 (spautin) (10 μM) or 3-MA (10 mM) for 4 hrs, and DMSO (1‰) was used as negative control. The cell lysates were harvested and analyzed by western blotting using: anti-Beclin1, anti-Atg14, anti-Vps34 and anti-UVRAG. Anti-α-tubulin was used as loading controls. F, 293T cells were treated with MBCQ or spautin in the presence of CHX to inhibit protein synthesis for indicated hrs and the cell lysates were analyzed by western blotting using anti-Beclin1. CHX (5 μM), MBCQ (10 μM), C43 (spautin) (10 μM). G, H4 cells were treated with Rapamycin (0.2 μM) with or without spautin (10 μM) or 3-MA (10 mM) for 4 hrs, and DMSO (1‰) was used as negative control. The cell lysates were harvested and analyzed by western blotting using: anti-Beclin1 and anti-LC3. Anti-α-tubulin was used as loading controls. H-M, 293T cells were transfected with indicated vectors. Twenty-four h after the transfection, the cells were treated with MBCQ (10 μM), C43 (spautin) (10 μM) or Rapamycin (0.2 μM) for an additional 4 h. The cell lysates were analyzed by western blotting using indicated antibodies.

FIG. 11 depicts results showing that selected cancer cell lines are sensitive to MBCQ and its active derivatives under glucose free condition. BT549 cells were treated with indicated concentrations of C43 for 24 h in normal DMEM (A) or under serum free condition (B). The cell viability was assayed by MTT or harvested for western blotting assay with anti-LC3 (C). MCF-7 cells were treated with DMSO (1‰), C43 (10 μM) in DMEM with (D) or without (E) glucose, for 12 h. The cell viability was assayed by MTT or images (F). And the cell lysates were analyzed by western blotting using anti-LC3 and α-tubulin was used as a loading control (G). Bcap-37 cells were treated with indicated concentrations of C43 for 24 h in normal DMEM (H) or under serum free condition (I). The cell viability was assayed by MTT or images (J) And the cell lysates treated with C43 for indicated time were analyzed by western blotting using anti-PARP (L) or anti-LC3 (M) and α-tubulin was used as a loading control. (K) Cell cycle profile of Bcap-37 treated with C43. Bcap-37 cells were treated with DMSO (0.1%) (left figure), C43 (10 μM) (right figure) for 12 h. The cells were then fixed with 70% ethanol, stained with propidium iodide (PI, 40 μg/mL) and treated with RNase enzyme (200 μg/mL) solution for 30 min in dark. Cell cycle profile and possible apoptotic death were statistics analyzed by flow cytometer.

FIG. 12 depicts the results showing of experiments showing that spautin does not induce apoptosis in non-cancer cells. A-B, MDCK cells were treated with DMSO (1‰) and spautin at indicated concentration in DMEM with or without glucose for 24 h. Cell survival as demonstrated by images (A) and MTT assay (B). C-D, Hs578Bst cells were treated with DMSO (1‰) and C43 as indicated concentration in DMEM with or without glucose for 24 h. Cell survival as demonstrated by images (C) and MTT assay (D).

FIG. 13 depicts results showing the effect of MBCQ and derivatives in vivo. (A) Mice were injected with rapamycin (10 mg/kg) alone as a positive control, or with C43 or MBCQ (40 mg/kg) intraperitoneally every hour for 4 h and then sacrificed at 5 th h. The autophagy levels in liver were analyzed by western blotting using anti-LC3 antibody. (B) C43 reduces the levels of autophagy induced by cerulein. Rats were injected intraperitoneally with cerulein (50 μg/kg) alone or with C43 (40 mg/kg) hourly for 4 times. The rats were sacrificed at one h after the last injection and the pancreas were isolated for western blotting analysis using anti-LC3 and anti-tubulin (as a control).

FIG. 14 depicts MBCQ derivatives that can inhibit autophagy. To calculate EC₅₀, H4-LC3 cells were seeded in 96 well-plates and cultured in the presence of compounds in different concentration for 24 h, and then fixed with polyformate and stained with 4,6-diamidino-2-phenylindole (DAPI, 3 μg/ml). Images data were collected with an ArrayScan HCS 4.0 Reader with a 20× objective (Cellomics, Pittsburgh, Pa.) for DAPI labeled nuclei and GFP-LC3, a marker for autophagy. The Spot Detector Bio-Application was used to acquire and analyze the images after optimization. Images of 1000 cells for each compound treatment were analyzed to obtain average cell number per field, fluorescence spot number, area and intensity per cell. DMSO and rapamycin were used as negative or positive control, respectively. The percentages of changes of LC3-GFP were calculated by dividing with that of DMSO treated samples. Each treatment was done in triplicate for mean and SD. The images were also analyzed using a conventional fluorescence microscope for visual inspection. The experiments were repeated three times

FIG. 15 depicts MBCQ derivatives with reduced or no ability to inhibit autophagy. To calculate EC₅₀, H4-LC3 cells were seeded in 96 well-plates and cultured in the presence of compounds in different concentration for 24 h, and then fixed with polyformate and stained with 4,6-diamidino-2-phenylindole (DAPI, 3 μg/ml). Images data were collected with an ArrayScan HCS 4.0 Reader with a 20× objective (Cellomics, Pittsburgh, Pa.) for DAPI labeled nuclei and GFP-LC3, a marker for autophagy. The Spot Detector Bio-Application was used to acquire and analyze the images after optimization. Images of 1000 cells for each compound treatment were analyzed to obtain average cell number per field, fluorescence spot number, area and intensity per cell. DMSO and rapamycin were used as negative or positive control, respectively. The percentages of changes of LC3-GFP were calculated by dividing with that of DMSO treated samples. Each treatment was done in triplicate for mean and SD. The images were also analyzed using a conventional fluorescence microscope for visual inspection. The experiments were repeated three times.

FIG. 16 depicts results of experiments showing that spautin promotes the degradation of Beclin1 through proteasomal pathway. A, 293T cells were transfected with GFP-Beclin1 and 24 hr after the transfection, the cells were treated with indicated compounds for an additional 24 hr. DMSO (1‰), MBCQ (10 μM), spautin (10 μM), NH4Cl (10 mM), MG132 (5 μM). The cell lysates were analyzed by western blotting using anti-GFP. B, 293T cells were transfected with GFP-Beclin1 and HA-Ub expression vectors. Twenty-four hours after the transfection, the cells were treated with MG132 or spautin for 24 hours. The cell lysates were immunoprecipitated with anti-GFP antibody and the immunocomplexes were analyzed by western blotting using anti-HA antibody.

FIG. 17 depicts the results of experiments demonstrating the effect of siRNA knockdown of USP3, USP10, USP13, USP16 and USP18 on the stability of selected autophagy proteins. H4 cells were transfected with indicated siRNAs for 72 hrs or treated with rapamycin (0.2 μM) or spautin (10 μM) for 4 hrs, and non-target siRNA (N. T. siRNA) was used as negative control. The cell lysates were harvested and analyzed by western blotting using (Left): antibodies specific for the indicated proteins. Anti-α-tubulin was used as loading controls.

FIG. 18 depicts the results of experiments demonstrating the effect of siRNA knockdown of USP3, USP10, USP13, USP16 and USP18 on the stability of USP proteins. H4 cells were transfected with indicated siRNAs for 72 hrs or treated with rapamycin (0.2 μM) or spautin (10 μM) for 4 hrs, and non-target siRNA (N. T. siRNA) was used as negative control. The cell lysates were harvested and analyzed by western blotting using (Left): antibodies specific for the indicated proteins. Anti-α-tubulin was used as loading controls.

FIG. 19 depicts the results of experiments demonstrating the effect of siRNA knockdown of USP3, USP10, USP13, USP16, USP18 and Beclin1 on the stability of P53. H4 cells were transfected with the indicated siRNAs (3 for each USP) and treated with Rapamycin (0.2 μM) for 4 hrs and DMSO (1%) was used as a negative control. The cell lysates were harvested and analyzed by western blotting using: anti-p53 antibody or other indicated antibody. Anti-α-tubulin was used as loading controls.

FIG. 20 depicts the results of experiments demonstrating that GFP-USP10 and Myc-USP13 could indeed interact and that the interaction was inhibited in spautin-treated cells. 293T cells were transfected with GFP-USP10 (lane 1-4), Myc-USP13 (lane 2-4), MG132 (lane 3-4) and/or spautin (lane 4). The lysates were immunoprecipitated with anti-GFP antibody and the immunocomplexes were analyzed by western blot with the indicated antibody.

FIG. 21 depicts the results of experiments demonstrating that flag-USP10 and GFP-Beclin1 could indeed interact and that the interaction was inhibited in spautin-treated cells. 293T cells were transfected with GFP-Beclin1 (lane 1), GFP-Beclin1 and Flag-USP10 (lane2-4) plasmids for 12 hours, incubated with MG132 (10 μM) with or without spautin (10 μM) for 4 h, the cell lysates were immunoprecipitated with anti-GFP antibody and the immunocomplexes were analyzed by western blotting using anti-Flag antibody.

FIG. 22 depicts the results of experiments demonstrating that flag-USP10 and GFP-Beclin1 could indeed interact and that the interaction was little effected in spautin-treated cells. 293T cells were transfected with GFP-Beclin1 (lane 1), GFP-Beclin1 and Myc-USP13 (lane2-4) plasmids for 12 hours, incubated with MG132 (10 μM) with or without spautin (10 μM) for 4 h, the cell lysates were immunoprecipitated with anti-GFP antibody and the immunocomplexes were analyzed by western blotting using anti-Myc antibody.

FIG. 23 depicts a ¹H NMR spectra of A9.

FIG. 24 depicts a ¹H NMR spectra of A30.

FIG. 25 depicts a ¹H NMR spectra of A36.

### DETAILED DESCRIPTION

Autophagy, a cellular catabolic process, plays an important role in promoting cell survival under metabolic stress condition by mediating lysosomal-dependent turnover of intracellular constituents for recycling. Inhibition of autophagy has been proposed as a possible new cancer therapy.

In an image-based screen for small molecule regulators of autophagy, an autophagy inhibitor, MBCQ, was identified. Extensive medicinal chemistry modification of MBCQ identified new derivatives, such as C43. It is disclosed that C43 inhibits autophagy with an IC₅₀ of about 0.8 μM in cell-based assays. It certain instances herein C43 is referred to as “spautin” (Specific and Potent AUtophagy Inhibitor). Derivatives of C43 with IC₅₀ of about 30 nM have also been prepared.

In addition, herein is disclosed that MBCQ and spautin can promote the degradation of Vps34 complexes (e.g., the type III PtdIns3 kinase complex involving Beclin1/Vps34/p150, whose product, PtdIns3P, is required for the onset of autophagy). It is further disclosed that ubiquitination and degradation of Vps34 complexes is regulated by a deubiquitinating protease complex which includes USP3, USP10, USP13, USP16 and USP18. The mechanism by which spautin inhibits autophagy is proposed herein to be the disruption of a deubiquitinating protease complex including USP10 and USP13 that is involved in regulating the turnover of Vps34 complexes in mammalian cells.

Further, it is disclosed herein that spautin is largely non-cytotoxic but induces apoptosis of a subset of cancer cells under starvation condition. Furthermore, it is disclosed herein that spautin inhibits autophagy in vivo in an animal model of pancreatitis.

### Definitions

For convenience, certain terms employed in the specification, examples, and appended claims are collected here. All definitions, as defined and used herein, supersede dictionary definitions, definitions in documents incorporated by reference, and/or ordinary meanings of the defined terms.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The definition of each expression, e.g., alkyl, m, n, and the like, when it occurs more than once in any structure, is intended to be independent of its definition elsewhere in the same structure.

It will be understood that “substitution” or “substituted with” includes the implicit proviso that such substitution is in accordance with permitted valence of the substituted atom and the substituent, and that the substitution results in a stable compound, e.g., a compound which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein below. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds. When “one or more” substituents are indicated, there may be, for example, 1, 2, 3, 4 or 5 substituents.

The term “lower” when appended to any of the groups listed below indicates that the group contains less than seven carbons (i.e., six carbons or less). For example “lower alkyl” refers to an alkyl group containing 1-6 carbons.

For purposes of this invention, the chemical elements are identified in accordance with the Periodic Table of the Elements, CAS version, Handbook of Chemistry and Physics, 67th Ed., 1986-87, inside cover.

The term “alkyl” means an aliphatic or cyclic hydrocarbon radical containing from 1 to 20, 1 to 15, or 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 2-methylcyclopentyl, and 1-cyclohexylethyl. The term “fluoroalkyl” means an alkyl wherein one or more hydrogens are replaced with fluorines.

The term “alkyoxy” means an alkyl group bound to the parent moiety through an oxygen. The term “fluoroalkoxy” means a fluoroalkyl group bound to the parent moiety through an oxygen.

### Selected Autophagy Inhibitors

One aspect of the invention relates to a compound represented by formula I:

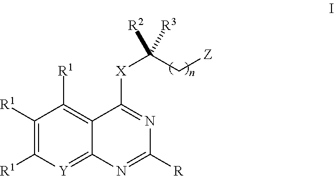

or a pharmaceutically acceptable salt, biologically active metabolite, solvate, hydrate, prodrug, enantiomer or stereoisomer thereof, wherein

n is 0, 1, 2, 3 or 4;

Y is —C(R¹)═ or —N═;

R is —H, lower alkyl, —CH₃, lower fluoroalkyl, —CH₂F, —CHF₂, —CF₃, —NO₂, —OH, —NH₂, —NH(lower alkyl), —N(lower alkyl)₂, or lower alkynyl;

R¹ is independently selected for each occurrence from the group consisting of —H, —F, —Cl, —Br, —I, —NO₂, —OH, —NH₂, —NH(lower alkyl), —N(lower alkyl)₂, —CH₃, —CF₃, —C(═O)(lower alkyl), —CN, —O(lower alkyl), —O(lower fluoroalkyl), —S(═O)(lower alkyl), —S(═O)₂(lower alkyl) and —C(═O)O(lower alkyl);

R² and R³ are independently selected from the group consisting of —H, lower alkyl, lower fluoroalkyl, lower alkynyl and hydroxyalkyl;

X is —O—, —S—, —N(H)—, —N(lower alkyl)-, —CH₂—, —CH₂CH₂—, —CH₂CH₂CH₂—, —CH₂CH₂CH₂CH₂—, —CH₂CH₂CH₂CH₂CH₂— or —CH₂CH₂CH₂CH₂CH₂CH₂—; and

Z is phenyl, pyridyl, vinyl, morphinyl, phenanthrolinyl, naphthyl, furyl or benzo[d]thiazolyl; and optionally substituted with one or more substitutents selected from the group consisting of —CH₃, lower alkyl, fluoroalkyl, —OCH₃, —OCF₃, lower fluoroalkoxy, —F, —Cl, —Br, —I, —NO₂, lower alkyoxy, —NH(lower alkyl), —N(lower alkyl)₂, —CF₃, and 3,4-methylene dioxy.

In certain embodiments, the invention relates to any of the aforementioned compounds and attendant definitions, provided that the compound is not

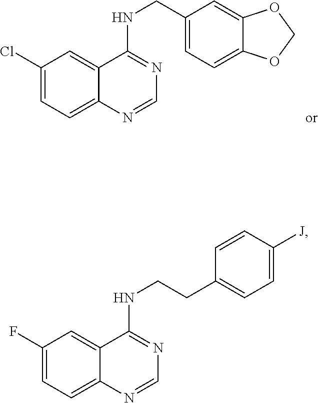

wherein J is Cl, OCHF₂, OCH₂CH₃, OCH₂CF₃, O(CH₂)₂CH₃, OCH(CH₃)₂, O(CH₂)₃CH₃, or O(cyclopentyl).

In certain embodiments, the invention relates to any of the aforementioned compounds and attendant definitions, wherein n is 0. In certain embodiments, the invention relates to any of the aforementioned compounds and attendant definitions, wherein n is 1. In certain embodiments, the invention relates to any of the aforementioned compounds and attendant definitions, wherein n is 2. In certain embodiments, the invention relates to any of the aforementioned compounds and attendant definitions, wherein n is 3. In certain embodiments, the invention relates to any of the aforementioned compounds and attendant definitions, wherein n is 4.

In certain embodiments, the invention relates to any of the aforementioned compounds and attendant definitions, wherein Y is —C(R¹)═.

In certain embodiments, the invention relates to any of the aforementioned compounds and attendant definitions, wherein Y is —C(H)═.

In certain embodiments, the invention relates to any of the aforementioned compounds and attendant definitions, wherein R is —N═.

In certain embodiments, the invention relates to any of the aforementioned compounds and attendant definitions, wherein R is —H.

In certain embodiments, the invention relates to any of the aforementioned compounds and attendant definitions, wherein R is lower alkyl or lower fluoroalkyl.

In certain embodiments, the invention relates to any of the aforementioned compounds and attendant definitions, wherein R is —CH₃.

In certain embodiments, the invention relates to any of the aforementioned compounds and attendant definitions, wherein R is —CH₂F, —CHF₂ or —CF₃. In certain embodiments, the invention relates to any of the aforementioned compounds and attendant definitions, wherein at only one R¹ is —H. In certain embodiments, the invention relates to any of the aforementioned compounds and attendant definitions, wherein only two R¹ are —H. In certain embodiments, the invention relates to any of the aforementioned compounds and attendant definitions, wherein only three R¹ are —H.

In certain embodiments, the invention relates to any of the aforementioned compounds and attendant definitions, wherein at least one R¹ is —NH₂, —Cl, —NO₂, —I, or —OMe. In certain embodiments, the invention relates to any of the aforementioned compounds and attendant definitions, wherein at one R¹ is —NH₂, —Cl, —NO₂, —I, or —OMe; and at least two R¹ are —H.

In certain embodiments, the invention relates to any of the aforementioned compounds and attendant definitions, wherein R² is —CH₃. In certain embodiments, the invention relates to any of the aforementioned compounds and attendant definitions, wherein R² is —H. In certain embodiments, the invention relates to any of the aforementioned compounds and attendant definitions, wherein R² is hydroxyalkyl.

In certain embodiments, the invention relates to any of the aforementioned compounds and attendant definitions, wherein R³ is —CH₃. In certain embodiments, the invention relates to any of the aforementioned compounds and attendant definitions, wherein R³ is —H. In certain embodiments, the invention relates to any of the aforementioned compounds and attendant definitions, wherein R³ is hydroxyalkyl.

In certain embodiments, the invention relates to any of the aforementioned compounds and attendant definitions, wherein R² is —CH_{3;} and R³ is H. In certain embodiments, the invention relates to any of the aforementioned compounds and attendant definitions, wherein R² is —H; and R³ is —H.

In certain embodiments, the invention relates to any of the aforementioned compounds and attendant definitions, wherein X is —O—, —S—, —N(H)—, —N(lower alkyl)- or —CH₂—. In certain embodiments, the invention relates to any of the aforementioned compounds and attendant definitions, wherein X is —N(H)— or —N(lower alkyl)-. In certain embodiments, the invention relates to any of the aforementioned compounds and attendant definitions, wherein X is —N(H)—.

In certain embodiments, the invention relates to any of the aforementioned compounds and attendant definitions, wherein n is 0 or 1; X is —N(H)—; R² is —H; R³ is —H; and R is —H.

In certain embodiments, the invention relates to any of the aforementioned compounds and attendant definitions, wherein Z is 4-pyridyl optionally substituted with one or more substitutents selected from the group consisting of —CH₃, lower alkyl, fluoroalkyl, —OCH₃, —OCF₃, lower fluoroalkoxy, —F, —Cl, —Br, —I, —NO₂, lower alkyoxy, —NH(lower alkyl), —N(lower alkyl)₂, —CF₃, and 3,4-methylene dioxy.

In certain embodiments, the invention relates to any of the aforementioned compounds and attendant definitions, wherein Z is morphinyl optionally substituted with one or more substitutents selected from the group consisting of —CH₃, lower alkyl, fluoroalkyl, —OCH₃, —OCF₃, lower fluoroalkoxy, —F, —Cl, —Br, —I, —NO₂, lower alkyoxy, —NH(lower alkyl), —N(lower alkyl)₂, —CF₃, and 3,4-methylene dioxy.

In certain embodiments, the invention relates to any of the aforementioned compounds and attendant definitions, wherein Z is 2-furyl, optionally substituted with one or more substitutents selected from the group consisting of —CH₃, lower alkyl, fluoroalkyl, —OCH₃, —OCF₃, lower fluoroalkoxy, —F, —Cl, —Br, —I, —NO₂, lower alkyoxy, —NH(lower alkyl), —N(lower alkyl)₂, —CF₃, and 3,4-methylene dioxy.

In certain embodiments, the invention relates to any of the aforementioned compounds and attendant definitions, wherein Z is 1-naphthyl or 2-napthyl optionally substituted with one or more substitutents selected from the group consisting of —CH₃, lower alkyl, fluoroalkyl, —OCH₃, —OCF₃, lower fluoroalkoxy, —F, —Cl, —Br, —I, —NO₂, lower alkyoxy, —NH(lower alkyl), —N(lower alkyl)₂, —CF₃, and 3,4-methylene dioxy.

In certain embodiments, the invention relates to any of the aforementioned compounds and attendant definitions, wherein Z is benzo[d]thiazol-5-yl or benzo[d]thiazol-6-yl optionally substituted with one or more substitutents selected from the group consisting of —CH₃, lower alkyl, fluoroalkyl, —OCH₃, —OCF₃, lower fluoroalkoxy, —F, —Cl, —Br, —I, —NO₂, lower alkyoxy, —NH(lower alkyl), —N(lower alkyl)₂, —CF₃, and 3,4-methylene dioxy. In certain embodiments, the invention relates to any of the aforementioned compounds and attendant definitions, wherein Z is phenyl optionally substituted with one or more substitutents selected from the group consisting of —CH₃, lower alkyl, fluoroalkyl, —OCH₃, —OCF₃, lower fluoroalkoxy, —F, —Cl, —Br, —I, —NO₂, lower alkyoxy, —NH(lower alkyl), —N(lower alkyl)₂, —CF₃, and 3,4-methylene dioxy.

In certain embodiments, the invention relates to any of the aforementioned compounds and attendant definitions, wherein n is 0 or 1; and Z is phenyl optionally substituted with one or more substitutents selected from the group consisting of —CH₃, lower alkyl, fluoroalkyl, —OCH₃, —OCF₃, lower fluoroalkoxy, —F, —Cl, —Br, —I, —NO₂, lower alkyoxy, —NH(lower alkyl), —N(lower alkyl)₂, —CF₃, and 3,4-methylene dioxy.

In certain embodiments, the invention relates to any of the aforementioned compounds and attendant definitions, wherein n is 0 or 1; X is —N(H)—; and Z is phenyl optionally substituted with one or more substitutents selected from the group consisting of —CH₃, lower alkyl, fluoroalkyl, —OCH₃, —OCF₃, lower fluoroalkoxy, —F, —Cl, —Br, —I, —NO₂, lower alkyoxy, —NH(lower alkyl), —N(lower alkyl)₂, —CF₃, and 3,4-methylene dioxy.

In certain embodiments, the invention relates to any of the aforementioned compounds and attendant definitions, wherein n is 0 or 1; X is —N(H)—; R² is —H; R³ is —H; and Z is phenyl optionally substituted with one or more substitutents selected from the group consisting of —CH₃, lower alkyl, fluoroalkyl, —OCH₃, —OCF₃, lower fluoroalkoxy, —F, —Cl, —Br, —I, —NO₂, lower alkyoxy, —NH(lower alkyl), —N(lower alkyl)₂, —CF₃, and 3,4-methylene dioxy.

In certain embodiments, the invention relates to any of the aforementioned compounds and attendant definitions, wherein n is 0 or 1; X is —N(H)—; R² is —H; R³ is -H; R is —H; and Z is phenyl optionally substituted with one or more substitutents selected from the group consisting of —CH₃, lower alkyl, fluoroalkyl, —OCH₃, —OCF₃, lower fluoroalkoxy, —F, —Cl, —Br, —I, —NO₂, lower alkyoxy, —NH(lower alkyl), —N(lower alkyl)₂, —CF₃, and 3,4-methylene dioxy.

One aspect of the invention relates to a compound represented by formula II:

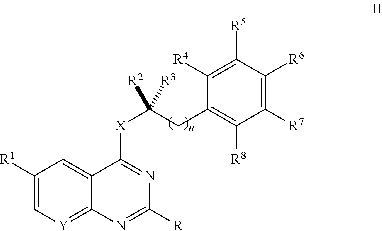

or a pharmaceutically acceptable salt, biologically active metabolite, solvate, hydrate, prodrug, enantiomer or stereoisomer thereof, wherein

n is 0, 1, 2, 3 or 4;

Y is —C(R¹)═ or —N═;

R is —H, lower alkyl, —CH₃, lower fluoroalkyl, —CH₂F, —CHF₂, or —CF₃;

R¹ is independently selected for each occurrence from the group consisting of —H, —CH₃, —F, —Cl, —Br, —I or —NO₂;

R² and R³ are independently selected from the group consisting of —H, —CH₃, —CH₂CH₃, —CH₂CH₂CH₃ or —CH(CH₃)₂;

R⁴, R⁵ and R⁸ are independently selected from the group consisting of —H, —CH₃, —CF₃, —OCH₃, —OCF₃, —F, —Cl, —Br or —I; and

R⁶ and R⁷ are independently selected from the group consisting of —H, —CH₃, —CF₃, —OCH₃, —OCF₃, —F, —Cl, —Br or —I; or R⁶ and R⁷ taken together are —OCH₂O—.

In certain embodiments, the invention relates to any of the aforementioned compounds and attendant definitions, provided that the compound is not

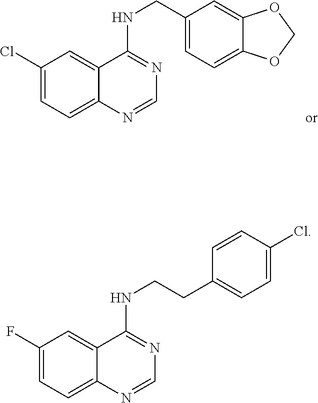

In certain embodiments, the invention relates to any of the aforementioned compounds and attendant definitions, wherein n is 0. In certain embodiments, the invention relates to any of the aforementioned compounds and attendant definitions, wherein n is 1. In certain embodiments, the invention relates to any of the aforementioned compounds and attendant definitions, wherein n is 2. In certain embodiments, the invention relates to any of the aforementioned compounds and attendant definitions, wherein n is 3. In certain embodiments, the invention relates to any of the aforementioned compounds and attendant definitions, wherein n is 4.

In certain embodiments, the invention relates to any of the aforementioned compounds and attendant definitions, wherein Y is —C(R¹)═.

In certain embodiments, the invention relates to any of the aforementioned compounds and attendant definitions, wherein Y is —C(H)═.

In certain embodiments, the invention relates to any of the aforementioned compounds and attendant definitions, wherein R is —N═.

In certain embodiments, the invention relates to any of the aforementioned compounds and attendant definitions, wherein R is —H.

In certain embodiments, the invention relates to any of the aforementioned compounds and attendant definitions, wherein R is lower alkyl or lower fluoroalkyl.

In certain embodiments, the invention relates to any of the aforementioned compounds and attendant definitions, wherein R is —CH₃.

In certain embodiments, the invention relates to any of the aforementioned compounds and attendant definitions, wherein R is —CH₂F, —CHF₂ or —CF₃.

In certain embodiments, the invention relates to any of the aforementioned compounds and attendant definitions, wherein R¹ is —F. In certain embodiments, the invention relates to any of the aforementioned compounds and attendant definitions, wherein R¹ is —Cl. In certain embodiments, the invention relates to any of the aforementioned compounds and attendant definitions, wherein R¹ is —Br. In certain embodiments, the invention relates to any of the aforementioned compounds and attendant definitions, wherein R¹ is —I. In certain embodiments, the invention relates to any of the aforementioned compounds and attendant definitions, wherein R¹ is —NO₂. In certain embodiments, the invention relates to any of the aforementioned compounds and attendant definitions, wherein R¹ is —CH₃.

In certain embodiments, the invention relates to any of the aforementioned compounds and attendant definitions, wherein R² is —H. In certain embodiments, the invention relates to any of the aforementioned compounds and attendant definitions, wherein R² is —CH₃, —CH₂CH₃, —CH₂CH₂CH₃ or —CH(CH₃)₂. In certain embodiments, the invention relates to any of the aforementioned compounds and attendant definitions, wherein R² is —CH₃.

In certain embodiments, the invention relates to any of the aforementioned compounds and attendant definitions, wherein R³ is —H. In certain embodiments, the invention relates to any of the aforementioned compounds and attendant definitions, wherein R³ is —CH₃, —CH₂CH₃, —CH₂CH₂CH₃ or —CH(CH₃)₂. In certain embodiments, the invention relates to any of the aforementioned compounds and attendant definitions, wherein R³ is —CH₃.

In certain embodiments, the invention relates to any of the aforementioned compounds and attendant definitions, wherein R⁴ is —H. In certain embodiments, the invention relates to any of the aforementioned compounds and attendant definitions, wherein R⁴ is —F. In certain embodiments, the invention relates to any of the aforementioned compounds and attendant definitions, wherein R⁴ is —Cl. In certain embodiments, the invention relates to any of the aforementioned compounds and attendant definitions, wherein R⁴ is —CH₃. In certain embodiments, the invention relates to any of the aforementioned compounds and attendant definitions, wherein R⁴ is —OCH₃.

In certain embodiments, the invention relates to any of the aforementioned compounds and attendant definitions, wherein R⁵ is —H. In certain embodiments, the invention relates to any of the aforementioned compounds and attendant definitions, wherein R⁵ is —F. In certain embodiments, the invention relates to any of the aforementioned compounds and attendant definitions, wherein R⁵ is —Cl. In certain embodiments, the invention relates to any of the aforementioned compounds and attendant definitions, wherein R⁵ is —CH₃. In certain embodiments, the invention relates to any of the aforementioned compounds and attendant definitions, wherein R⁵ is —OCH₃.

In certain embodiments, the invention relates to any of the aforementioned compounds and attendant definitions, wherein R⁶ is —H, —F, —Cl, —Br or —I. In certain embodiments, the invention relates to any of the aforementioned compounds and attendant definitions, wherein R⁶ is —H. In certain embodiments, the invention relates to any of the aforementioned compounds and attendant definitions, wherein R⁶ is —F. In certain embodiments, the invention relates to any of the aforementioned compounds and attendant definitions, wherein R⁶ is —Cl. In certain embodiments, the invention relates to any of the aforementioned compounds and attendant definitions, wherein R⁶ is —Br. In certain embodiments, the invention relates to any of the aforementioned compounds and attendant definitions, wherein R⁶ is —CH₃. In certain embodiments, the invention relates to any of the aforementioned compounds and attendant definitions, wherein R⁶ is —CF₃. In certain embodiments, the invention relates to any of the aforementioned compounds and attendant definitions, wherein R⁶ is —OCH₃. In certain embodiments, the invention relates to any of the aforementioned compounds and attendant definitions, wherein R⁶ is —OCF₃.

In certain embodiments, the invention relates to any of the aforementioned compounds and attendant definitions, wherein R⁶ and R⁷taken together are —OCH₂O—.

In certain embodiments, the invention relates to any of the aforementioned compounds and attendant definitions, wherein R⁷ is —H, —F, —Cl, —Br or —I. In certain embodiments, the invention relates to any of the aforementioned compounds and attendant definitions, wherein R⁷ is —H.

In certain embodiments, the invention relates to any of the aforementioned compounds and attendant definitions, wherein R⁸ is —H.

One aspect of the invention relates to a compound, or a pharmaceutically acceptable salt, biologically active metabolite, solvate, hydrate, prodrug, enantiomer or stereoisomer thereof, selected from the group consisting of

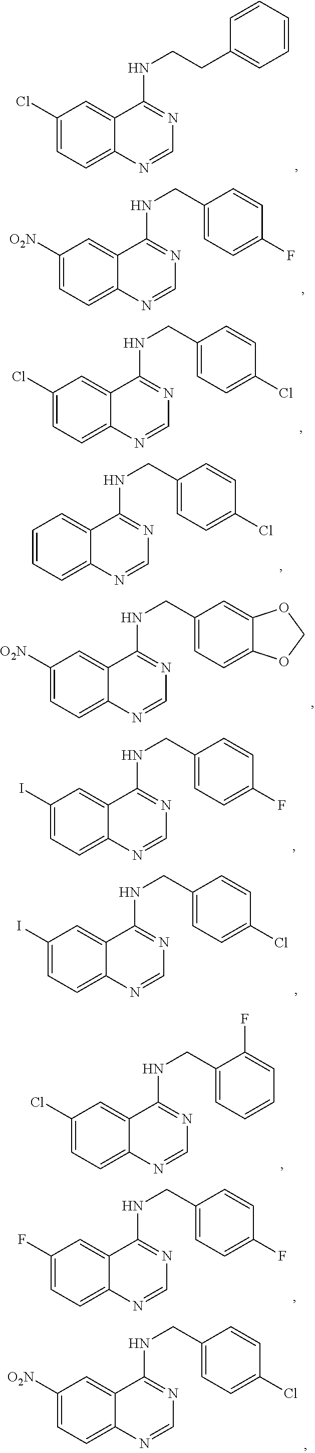

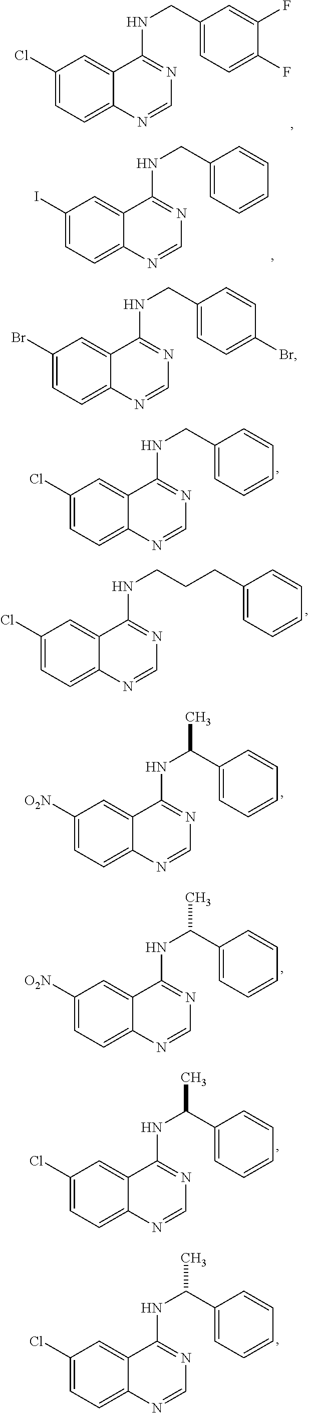

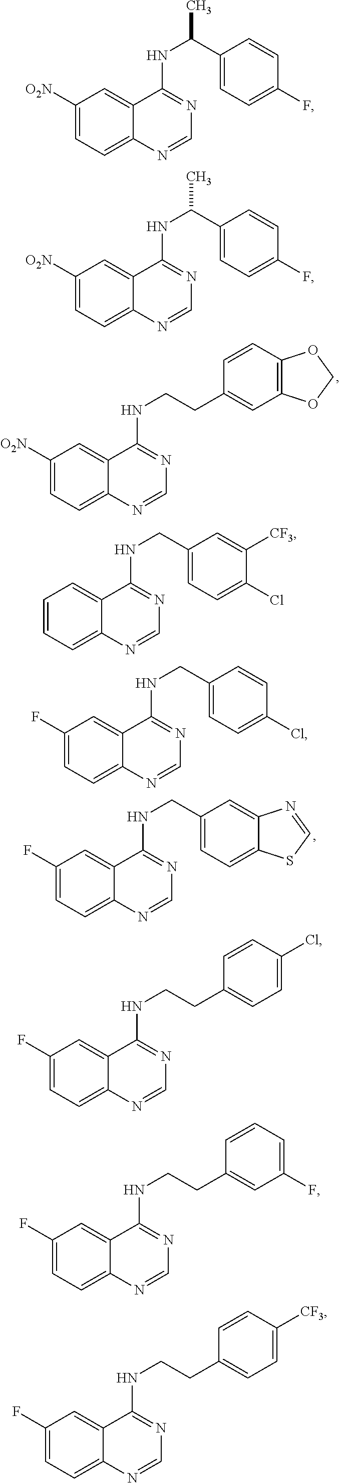

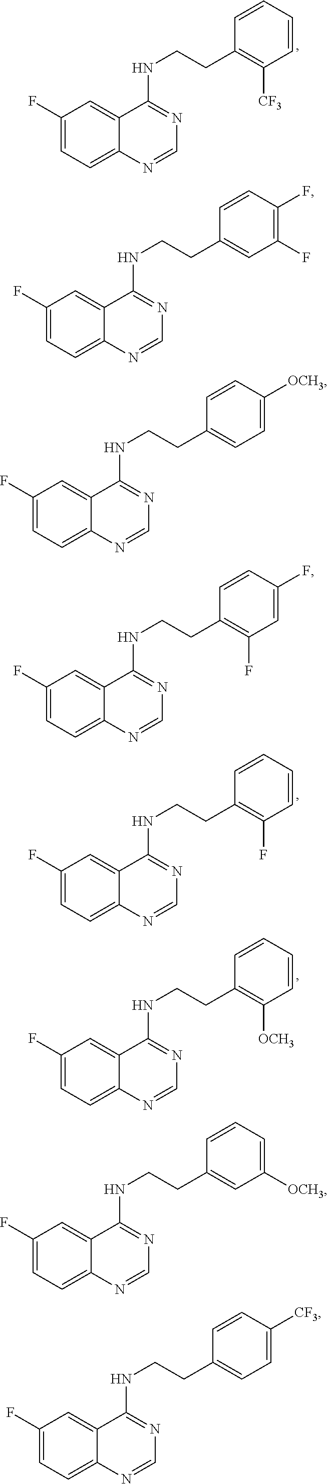

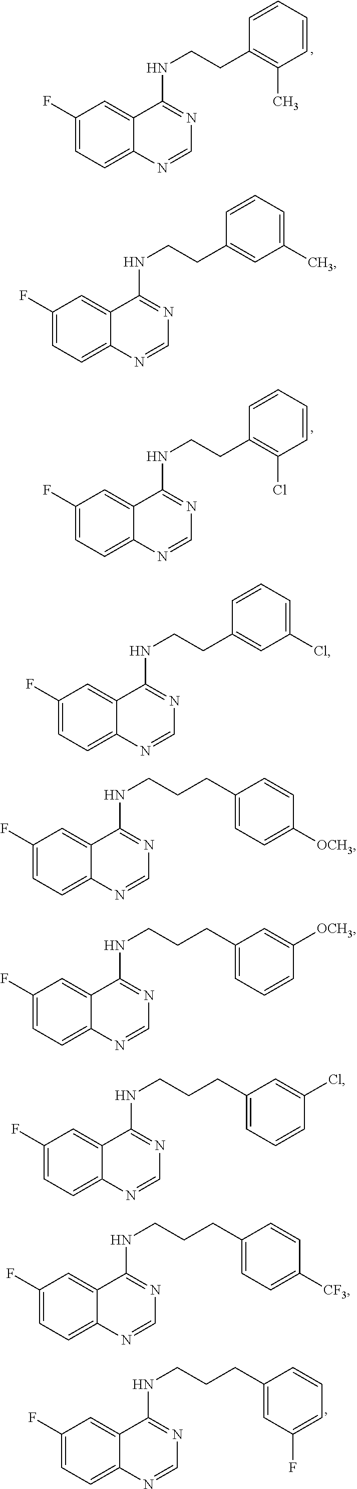

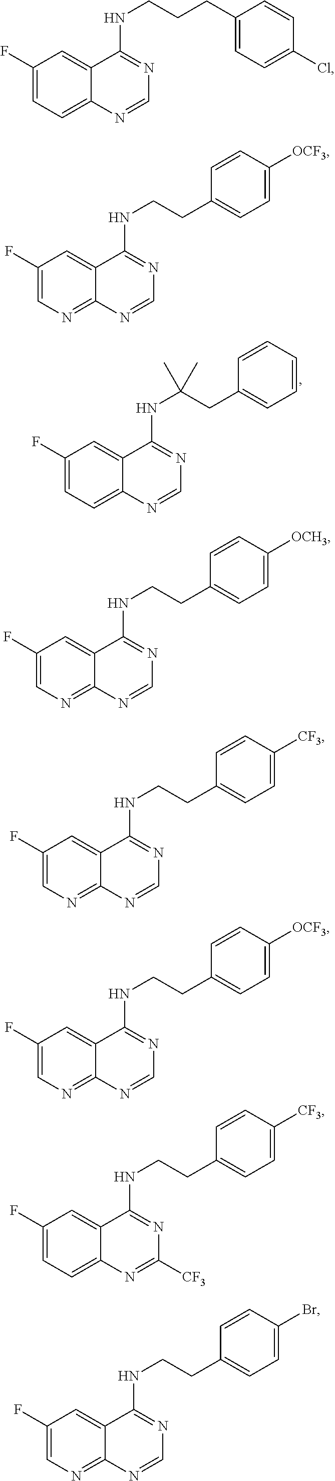

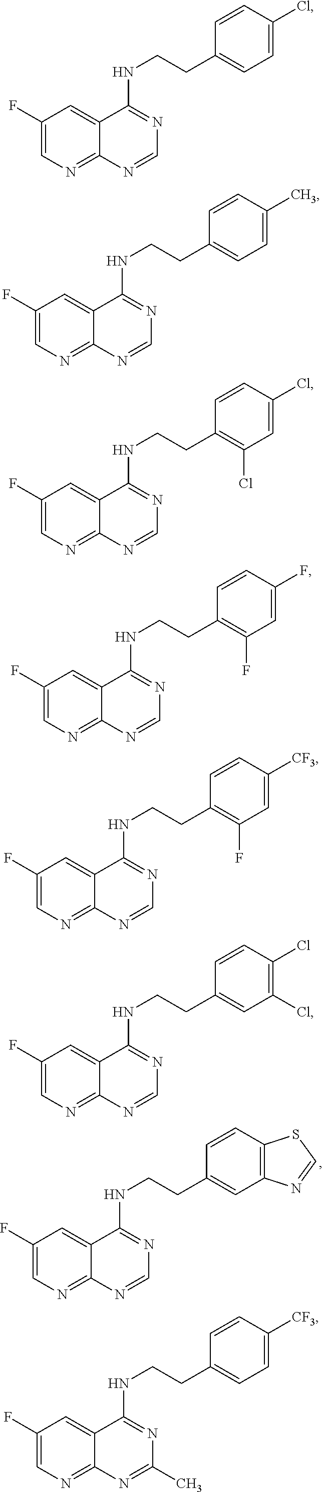

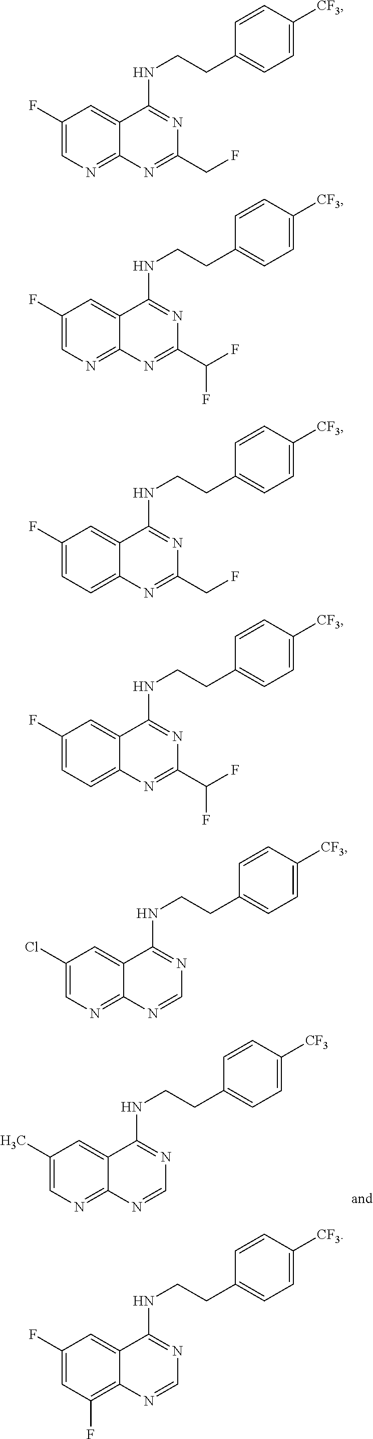

One aspect of the invention relates to

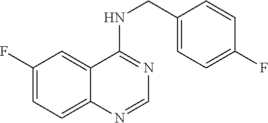

or a pharmaceutically acceptable salt, biologically active metabolite, solvate, hydrate, prodrug, enantiomer or stereoisomer thereof.

In certain embodiments, the invention relates to any of the aforementioned compounds and attendant definitions, wherein the compound is an autophagy inhibitor; and the EC₅₀ of the autophagy inhibitor is less than about 100 nM.

In certain embodiments, the invention relates to any one of the aforementioned compounds, wherein the compound inhibits autophagy with an IC₅₀ of less than about 10 μM. In certain embodiments, the invention relates to any one of the aforementioned compounds, wherein the compound inhibits autophagy with an IC₅₀ of less than about 5 μM. In certain embodiments, the invention relates to any one of the aforementioned compounds, wherein the compound inhibits autophagy with an IC₅₀ of less than about 1 μM. In certain embodiments, the invention relates to any one of the aforementioned compounds, wherein the compound inhibits autophagy with an IC₅₀ of less than about 750 nM. In certain embodiments, the invention relates to any one of the aforementioned compounds, wherein the compound inhibits autophagy with an IC₅₀ of less than about 500 nM. In certain embodiments, the invention relates to any one of the aforementioned compounds, wherein the compound inhibits autophagy with an IC₅₀ of less than about 250 nM. In certain embodiments, the invention relates to any one of the aforementioned compounds, wherein the compound inhibits autophagy with an IC₅₀ of less than about 100 nM.

In certain embodiments, the invention relates to any one of the aforementioned compounds, wherein the compound is an inhibitor of autophagy; and the compound does not inhibit PDE5.

In certain embodiments, the invention relates to any one of the aforementioned compounds, wherein the compound inhibits both autophagy and PDE5 the compound has an autophagy IC₅₀ of between about 0.001 μM and about 10 μM; and the ratio of the PDE5 IC₅₀ to the autophagy IC₅₀ is between about 10 and about 50. In certain embodiments, the invention relates to any one of the aforementioned compounds, wherein the compound inhibits both autophagy and PDE5; the compound has an autophagy IC₅₀ of between about 0.001 μM and about 10 μM; and the ratio of the PDE5 IC₅₀ to the autophagy IC₅₀ is between about 50 and about 100. In certain embodiments, the invention relates to any one of the aforementioned compounds, wherein the compound inhibits both autophagy and PDE5; the compound has an autophagy IC₅₀ of between about 0.001 μM and about 10 μM; and the ratio of the PDE5 IC₅₀ to the autophagy IC₅₀ is between about 100 and about 1,000.

Certain compounds of the invention which have acidic substituents may exist as salts with pharmaceutically acceptable bases. The present invention includes such salts. Examples of such salts include sodium salts, potassium salts, lysine salts and arginine salts. These salts may be prepared by methods known to those skilled in the art.

Certain compounds of the invention and their salts may exist in more than one crystal form and the present invention includes each crystal form and mixtures thereof.

Certain compounds of the invention and their salts may also exist in the form of solvates, for example hydrates, and the present invention includes each solvate and mixtures thereof.

Certain compounds of the invention may contain one or more chiral centers, and exist in different optically active forms. When compounds of the invention contain one chiral center, the compounds exist in two enantiomeric forms and the present invention includes both enantiomers and mixtures of enantiomers, such as racemic mixtures. The enantiomers may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step may be used to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a compound of the invention contains more than one chiral center, it may exist in diastereoisomeric forms. The diastereoisomeric compounds may be separated by methods known to those skilled in the art, for example chromatography or crystallization and the individual enantiomers may be separated as described above. The present invention includes each diastereoisomer of compounds of the invention and mixtures thereof.

Certain compounds of the invention may exist in different tautomeric forms or as different geometric isomers, and the present invention includes each tautomer and/or geometric isomer of compounds of the invention and mixtures thereof.

Certain compounds of the invention may exist in different stable conformational forms which may be separable. Torsional asymmetry due to restricted rotation about an asymmetric single bond, for example because of steric hindrance or ring strain, may permit separation of different conformers. The present invention includes each conformational isomer of compounds of the invention and mixtures thereof.

Certain compounds of the invention may exist in zwitterionic form and the present invention includes each zwitterionic form of compounds of the invention and mixtures thereof.

As used herein the term “pro-drug” refers to an agent which is converted into the parent drug in vivo by some physiological chemical process (e.g., a prodrug on being brought to the physiological pH is converted to the desired drug form). Pro-drugs are often useful because, in some situations, they may be easier to administer than the parent drug. They may, for instance, be bioavailable by oral administration whereas the parent drug is not. The prodrug may also have improved solubility in pharmacological compositions over the parent drug. An example, without limitation, of a pro-drug would be a compound of the present invention wherein it is administered as an ester (the “pro-drug”) to facilitate transmittal across a cell membrane where water solubility is not beneficial, but then it is metabolically hydrolyzed to the carboxylic acid once inside the cell where water solubility is beneficial. Pro-drugs have many useful properties. For example, a pro-drug may be more water soluble than the ultimate drug, thereby facilitating intravenous administration of the drug. A pro-drug may also have a higher level of oral bioavailability than the ultimate drug. After administration, the prodrug is enzymatically or chemically cleaved to deliver the ultimate drug in the blood or tissue.

Exemplary pro-drugs release an amine of a compound of the invention wherein the free hydrogen of an amine is replaced by (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanoyl, arylactyl and α-aminoacyl, or α-aminoacyl-α-aminoacyl wherein said α-aminoacyl moieties are independently any of the naturally occurring L-amino acids found in proteins, —P(O)(OH)₂, —P(O)(O(C₁-C₆)alkyl)₂ or glycosyl (the radical resulting from detachment of the hydroxyl of the hemiacetal of a carbohydrate).

### Pharmaceutical Compositions

One or more compounds of this invention can be administered to a human patient by themselves or in pharmaceutical compositions where they are mixed with biologically suitable carriers or excipient(s) at doses to treat or ameliorate a disease or condition as described herein. Mixtures of these compounds can also be administered to the patient as a simple mixture or in suitable formulated pharmaceutical compositions. For example, one aspect of the invention relates to pharmaceutical composition comprising a therapeutically effective dose of a compound of formula I or II, or a pharmaceutically acceptable salt, biologically active metabolite, solvate, hydrate, prodrug, enantiomer or stereoisomer thereof; and a pharmaceutically acceptable diluent or carrier.

As used herein, a therapeutically effective dose refers to that amount of the compound or compounds sufficient to result in the prevention or attenuation of a disease or condition as described herein. Techniques for formulation and administration of the compounds of the instant application may be found in references well known to one of ordinary skill in the art, such as “Remington's Pharmaceutical Sciences,” Mack Publishing Co., Easton, Pa., latest edition.

Suitable routes of administration may, for example, include oral, eyedrop, rectal, transmucosal, topical, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one may administer the compound in a local rather than a systemic manner, for example, via injection of the compound directly into an edematous site, often in a depot or sustained release formulation.

Furthermore, one may administer the drug in a targeted drug delivery system, for example, in a liposome coated with endothelial cell-specific antibody.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the active compound with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds can be formulated for parenteral administration by injection, e.g., bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly or by intramuscular injection). Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Alternatively, other delivery systems for hydrophobic pharmaceutical compounds may be employed. Liposomes and emulsions are well known examples of delivery vehicles or carriers for hydrophobic drugs. Certain organic solvents such as dimethysulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions (i.e., pharmaceutically acceptable salts). A “pharmaceutically acceptable salt” means any non-toxic salt that, upon administration to a recipient, is capable of providing, either directly or indirectly, a compound or a prodrug of a compound of this invention. A “pharmaceutically acceptable counterion” is an ionic portion of a salt that is not toxic when released from the salt upon administration to a recipient. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free base forms.

Acids commonly employed to form pharmaceutically acceptable salts include inorganic acids such as hydrogen bisulfide, hydrochloric, hydrobromic, hydroiodic, sulfuric and phosphoric acid, as well as organic acids such as para-toluenesulfonic, salicylic, tartaric, bitartaric, ascorbic, maleic, besylic, fumaric, gluconic, glucuronic, formic, glutamic, methanesulfonic, ethanesulfonic, benzenesulfonic, lactic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, .beta.-hydroxybutyrate, glycolate, maleate, tartrate, methanesulfonate, propanesulfonate, naphthalene-1-sulfonate, naphthalene-2-sulfonate, mandelate and the like salts. Preferred pharmaceutically acceptable acid addition salts include those formed with mineral acids such as hydrochloric acid and hydrobromic acid, and especially those formed with organic acids such as maleic acid.

Suitable bases for forming pharmaceutically acceptable salts with acidic functional groups include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N-di alkyl-N-(hydroxy alkyl)-amines, such as N,N-dimethyl-N-(2-hydroxyethyl)amine, or tri-(2-hydroxyethyl)amine; N-methyl-D-glucamine; and amino acids such as arginine, lysine, and the like.

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art.

### Selected Methods of Use

One aspect the invention provides a method for inhibiting autophagy in a subject for whom inhibition of autophagy is beneficial, comprising administering to the subject a compound of the invention such that autophagy activity in the subject is altered and treatment or prevention is achieved. In certain embodiments, the subject is a human.

The term “treating” as used herein, encompasses the administration and/or application of one or more compounds described herein, to a subject, for the purpose of providing prevention of or management of, and/or remedy for a condition. “Treatment” for the purposes of this disclosure, may, but does not have to, provide a cure; rather, “treatment” may be in the form of management of the condition. When the compounds described herein are used to treat unwanted proliferating cells, including cancers, “treatment” includes partial or total destruction of the undesirable proliferating cells with minimal destructive effects on normal cells. A desired mechanism of treatment of unwanted rapidly proliferating cells, including cancer cells, at the cellular level is apoptosis.

The term “preventing” as used herein includes either preventing or slowing the onset of a clinically evident unwanted cell proliferation altogether or preventing or slowing the onset of a preclinically evident stage of unwanted rapid cell proliferation in individuals at risk. Also intended to be encompassed by this definition is the prevention or slowing of metastasis of malignant cells or to arrest or reverse the progression of malignant cells. This includes prophylactic treatment of those at risk of developing precancers and cancers. Also encompassed by this definition is the prevention or slowing of restenosis in subjects that have undergone angioplasty or a stent procedure.

The term “subject” for purposes of treatment includes any human or animal subject who has been diagnosed with, has symptoms of, or is at risk of developing a disorder wherein inhibition of autophagy would be beneficial. For methods of prevention the subject is any human or animal subject. To illustrate, for purposes of prevention, a subject may be a human subject who is at risk of or is genetically predisposed to obtaining a disorder characterized by unwanted, rapid cell proliferation, such as cancer. The subject may be at risk due to exposure to carcinogenic agents, being genetically predisposed to disorders characterized by unwanted, rapid cell proliferation, and so on. Besides being useful for human treatment, the compounds described herein are also useful for veterinary treatment of mammals, including companion animals and farm animals, such as, but not limited to dogs, cats, horses, cows, sheep, and pigs.

One aspect of the invention relates to a method of treating or preventing cancer, comprising the step of administering to a subject in need thereof a therapeutically effective amount of one or more compounds of formula I or II, or a pharmaceutically acceptable salt, biologically active metabolite, solvate, hydrate, prodrug, enantiomer or stereoisomer thereof.

Suppression of autophagy has been proposed to be a new anticancer therapy by promoting radiosensitization and chemosensitization. In an animal model of cancer therapy, inhibition of therapy-induced autophagy either with shRNA against a key autophagy gene ATG5 or with anti-malarial drug chloroquine enhanced cell death and tumor regression of Myc-driven tumors in which either activated p53 or alkylating chemotherapy was used to drive tumor cell death (Amaravadi, R. K., et al., Autophagy inhibition enhances therapy-induced apoptosis in a Myc-induced model of lymphoma. J Clin Invest, 2007. 117(2): p. 326-36). Chloroquine causes a dose-dependent accumulation of large autophagic vesicles and enhances alkylating therapy-induced cell death to a similar degree as knockdown of ATG5. In the case of chronic myelogenous leukemia (CML), chloroquine markedly enhanced death of a CML cell line, K562, induced by imatinib. Furthermore, imatinib-resistant cell lines, BaF3/T315I and BaF3/E255K, can be induced to die by co-treatment with imatinib and chloroquine. These studies suggest that inhibiting autophagy may potentiate conventional chemotherapy.

The National Cancer Institute alphabetical list of cancer includes: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macroglobulinemia; and Wilms' Tumor. The methods of the present invention may be useful to treat such types of cancer.

Another aspect of the invention relates to a method of treating or preventing acute pancreatitis, comprising the step of administering to a subject in need thereof a therapeutically effective amount of one or more compounds of formula I or II, or a pharmaceutically acceptable salt, biologically active metabolite, solvate, hydrate, prodrug, enantiomer or stereoisomer thereof.

Pancreatitis is an inflammation of the pancreas mediated by the release of digestive enzymes that eventually lead to the destruction of the organ itself. Pancreatitis can be a severe, life-threatening illness with many complications. In severe cases, bleeding, tissue damage to the heart, lungs and kidneys, and infection may occur. About 80,000 cases of acute pancreatitis occur annually in the United States; about 20 percent of them are severe. There is no known treatment for pancreatitis. The current approaches for managing pancreatitis involve waiting for it to resolve on its own and the treatment of heart, lungs and kidney complications if that occur.

Autophagy has been shown to play an important role in mediating cellular damage induced by acute pancreatitis. Autodigestion of the pancreas by its own prematurely activated digestive proteases is believed to be important for the onset of acute pancreatitis. Although lysosomal hydrolases are known to play a key role in pancreatic trypsinogen activation, it remains unclear where and how trypsinogen meets these lysosomal enzymes. Recently, autophagy has been proposed to play a key role in the release of pancreatitic digestive enzymes in animal models of pancreatitis (Hashimoto, D., et al., Involvement of autophagy in trypsinogen activation within the pancreatic acinar cells. J Cell Biol, 2008. 181(7): p. 1065-72; and Ohmuraya, M. and K. Yamamura, Autophagy and acute pancreatitis: a novel autophagy theory for trypsinogen activation. Autophagy, 2008. 4(8): p. 1060-2.) In Atg5−/− mice, which are defective for a key autophagy gene Atg5, the severity of acute pancreatitis induced by cerulein is greatly reduced with a significantly decreased level of trypsinogen activation. Thus, activation of autophagy may exert a detrimental effect in pancreatic acinar cells by mediating the activation of trypsinogen to trypsin. Inhibition of autophagy may provide a unique opportunity for blocking trypsinogen activation in acute pancreatitis. Development of an autophagy inhibitor may provide a first-in-class inhibitor for acute pancreatitis.

Another aspect of the invention relates to a method of treating or preventing a disease caused by an intracellular pathogen, comprising the step of administering to a subject in need thereof a therapeutically effective amount of one or more compounds of formula I or II, or a pharmaceutically acceptable salt, biologically active metabolite, solvate, hydrate, prodrug, enantiomer or stereoisomer thereof. See, for example, US Patent Application Publication No. 2009/0111799 to Chen et al. (hereby incorporated by reference in its entirety).

Recent studies have established a role for autophagy in cellular defense against intracellular pathogens including bacteria, such as Mycobacterium tuberculosis, Streptococcus pyogenes, Shigella spp. and Salmonella typhimurium, as well as viruses and protozoa which use autophagosomes to proliferate. The execution of autophagy is regulated by upstream signal transduction systems that are influenced by largely physiological factors such as nutrient status, growth factors/cytokines, and hypoxia. The pharmacological induction of autophagy is a therapeutic strategy in which this effector of innate immunity would be triggered or amplified to defend against intracellular pathogens.

Another aspect of the invention relates to a method of inactivating a deubiquitinating protease complex comprising the step of contacting the deubiquitinating protease complex with one or more compounds of formula I or II; wherein the deubiquitinating protease complex comprises USP3 and USP10. Such methods can be used to ameliorate any condition which is caused by or potentiated by the activity of the deubiquitinating protease complex.

### Combination Therapy

In one aspect of the invention, a compound of the invention, or a pharmaceutically acceptable salt thereof, can be used alone or in combination with another therapeutic agent to treat diseases such cancer and pancreatitis. It should be understood that the compounds of the invention can be used alone or in combination with an additional agent, e.g., a therapeutic agent, said additional agent being selected by the skilled artisan for its intended purpose. For example, the additional agent can be a therapeutic agent that is art-recognized as being useful to treat the disease or condition being treated by the compound of the present invention. The additional agent also can be an agent that imparts a beneficial attribute to the therapeutic composition e.g., an agent that affects the viscosity of the composition.

The combination therapy contemplated by the invention includes, for example, administration of a compound of the invention, or a pharmaceutically acceptable salt thereof, and additional agent(s) in a single pharmaceutical formulation as well as administration of a compound of the invention, or a pharmaceutically acceptable salt thereof, and additional agent(s) in separate pharmaceutical formulations. In other words, co-administration shall mean the administration of at least two agents to a subject so as to provide the beneficial effects of the combination of both agents. For example, the agents may be administered simultaneously or sequentially over a period of time.

It should further be understood that the combinations included within the invention are those combinations useful for their intended purpose. The agents set forth below are illustrative for purposes and not intended to be limited. The combinations, which are part of this invention, can be the compounds of the present invention and at least one additional agent selected from the lists below. The combination can also include more than one additional agent, e.g., two or three additional agents if the combination is such that the formed composition can perform its intended function.

For example, one aspect of the invention relates to the use of small molecule autophagy inhibitors (e.g. those of formula I or II) in combination with an anti-angiogenesis inhibitors for the treatment of cancers. It is known that anti-angiogenesis inhibitors have the promise to inhibit tumor growth by suppressing the growth of blood vessels in tumors which are required for supporting tumor survival and growth. For example, the angiostatic agent endostatin and related chemicals can suppress the building of blood vessels and reduce tumor growth. Several hundred clinical trials of anti-angiogenesis drugs are now under way. In tests with patients, anti-angiogenesis therapies are able to suppress tumor growth with relatively few side effects. However, anti-angiogenesis therapy alone may not be insufficient to prolong patient survival; combination with a conventional chemotherapy may therefore be beneficial. Specifically, autophagy inhibitors may provide a new option to work alone or in combination with anti-angiogenesis therapy.

Endostatin has been shown to induce autophagy in endothelial cells by modulating Beclin 1 and beta-catenin levels (Nguyen, T. M., et al., Endostatin induces autophagy in endothelial cells by modulating Beclin 1 and beta-catenin levels. J Cell Mol Med, 2009). As disclosed herein, it has been found that inhibition of autophagy selectively kills a subset of cancer cells under starvation condition. Therefore, it is proposed that anti-angiogenesis therapy may induce additional metabolic stress to sensitize cancer cells to autophagy inhibitors, which are not normally cytotoxic. Thus, a combination of anti-angiogenesis therapy and anti-autophagy therapy may provide a new option for treatment of cancers without cytotoxicity to normal cells (Ramakrishnan, S., et al., Autophagy and angiogenesis inhibition. Autophagy, 2007. 3(5): p. 512-5).

Non-limiting examples of anti-angiogenesis agents with which a compound of the invention of the invention can be combined include, for example, the following: bevacizumab (Avastin®), carboxyamidotriazole, TNP-470, CM101, IFN-α, IL-12, platelet factor-4, suramin, SU5416, thrombospondin, VEGFR antagonists, angiostatic steroids with heparin, Cartilage-Derived Angiogenesis Inhibitory Factor, matrix metalloproteinase inhibitors, angiostatin, endostatin, 2-methoxyestradiol, tecogalan, thrombospondin, prolactin, αVβ3 inhibitors and linomide.

In addition, as described in US Patent Application Publication No. 2008/0269259 to Thompson et al. (hereby incorporated by reference in its entirety), autophagy inhibitors can be used to treat a subject who has been identified as having a glycolysis dependent cancer by combining one or more autophagy inhibitors with one or more anti-cancer compounds which converts glycolysis dependent cancer to cells incapable of glycolysis. Examples of anti-cancer compounds which convert glycolysis dependent cancer to cells incapable of glycolysis: Alkylating Agents; Nitrosoureas; Antitumor Antibiotics; Corticosteroid Hormones; Anti-estrogens; Aromatase Inhibitors; Progestins; Anti-androgens; LHRH agonists; Kinase Inhibitors; and Antibody therapies; for example, busulfan, cisplatin, carboplatin, chlorambucil, cyclophosphamide, ifosfamide, dacarbazine (DTIC), mechlorethamine (nitrogen mustard), melphalan, carmustine (BCNU), lomustine (CCNU), dactinomycin, daunorubicin, doxorubicin (Adriamycin), idarubicin, mitoxantrone, prednisone, dexamethasone, tamoxifen, fulvestrant, anastrozole, letrozole, megestrol acetate, bicalutamide, flutamide. leuprolide, goserelin, gleevac, Iressa, Tarceva, Herceptin, Avastin, L-asparaginase and tretinoin.

### Dosage

As used herein, a “therapeutically effective amount” or “therapeutically effective dose” is an amount of a compound of the invention or a combination of two or more such compounds, which inhibits, totally or partially, the progression of the condition or alleviates, at least partially, one or more symptoms of the condition. A therapeutically effective amount can also be an amount which is prophylactically effective. The amount which is therapeutically effective will depend upon the patient's size and gender, the condition to be treated, the severity of the condition and the result sought. For a given patient, a therapeutically effective amount can be determined by methods known to those of skill in the art.

For any compound used in a method of the present invention, the therapeutically effective dose can be estimated initially from cellular assays. For example, a dose can be formulated in cellular and animal models to achieve a circulating concentration range that includes the IC₅₀ as determined in cellular assays (i.e., the concentration of the test compound which achieves a half-maximal inhibition). In some cases it is appropriate to determine the IC₅₀ in the presence of 3 to 5% serum albumin since such a determination approximates the binding effects of plasma protein on the compound. Such information can be used to more accurately determine useful doses in humans.

A therapeutically effective dose refers to that amount of the compound that results in amelioration of symptoms in a patient. Toxicity and therapeutic efficacy of such compounds can be determined by standard pharmaceutical procedures in cell cultures or experimental animals, e.g., for determining the maximum tolerated dose (MTD) and the ED₅₀ (effective dose for 50% maximal response). The dose ratio between toxic and therapeutic effects is the therapeutic index and it can be expressed as the ratio between MTD and ED₅₀. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p 1). In the treatment of crises, the administration of an acute bolus or an infusion approaching the MTD may be required to obtain a rapid response.

Dosage amount and interval may be adjusted individually to provide plasma levels of the active moiety which are sufficient to maintain the kinase modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using the MEC value. Compounds should be administered using a regimen which maintains plasma levels above the MEC for 10-90% of the time, preferably between 30-90% and most preferably between 50-90% until the desired amelioration of symptoms is achieved. In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

### Kits

The compounds and compositions of the invention (e.g., compounds and compositions of formula I or II) may, if desired, be presented in a kit (e.g., a pack or dispenser device). The pack may for example comprise metal or plastic foil, such as a blister pack. The pack or dispenser device may be accompanied by instructions for use of the compound in any method described herein. Compositions comprising a compound of the invention formulated in a compatible pharmaceutical carrier may also be prepared, placed in an appropriate container, and labelled for treatment of an indicated condition. Instructions for use may also be provided.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### Isolation of a Small Molecule Inhibitors of Autophagy

To explore the mechanism of autophagy and identify additional small molecules that can activate it, a high-throughput image-based screen for autophagy regulators was developed. This system takes advantage of the localization of light chain 3 coupled to GFP (LC3-GFP) to autophagosomal membrane upon induction of autophagy (Zhang, L., Yu, J., Pan, H., Hu, P., Hao, Y., Cai, W., Zhu, H., Yu, A. D., Xie, X., Ma, D., et al. (2007). Small molecule regulators of autophagy identified by an image-based high-throughput screen. Proc Natl Acad Sci USA 104, 19023-19028). Mammalian LC3, the ortholog of yeast ATG8, has been shown to mark autophagosome membrane specifically (Kabeya, Y., Mizushima, N., Ueno, T., Yamamoto, A., Kirisako, T., Noda, T., Kominami, E., Ohsumi, Y., and Yoshimori, T. (2000). LC3, a mammalian homologue of yeast Apg8p, is localized in autophagosome membranes after processing. EMBO J 19, 5720-5728; and Mizushima, N., and Yoshimori, T. (2007). How to interpret LC3 immunoblotting. Autophagy 3, 542-545). The number of LC3-GFP-positive autophagosomes per cell is very low under normal growth conditions but is rapidly increased upon serum starvation or the addition of rapamycin. Compounds that increase cellular levels of LC3-GFP, however, are not necessarily able to increase degradative activity of autophagy. Instead, the increases of LC3-GFP may be associated with cell death or may be a result of lysosomal defect and thus associated with blockage of autophagy.

In a screen of 480 known bioactive compounds, a LC3-GFP-based high throughput image screen was coupled with a low throughput assay for long-lived protein degradation which allowed for the identification compounds which could specifically induce autophagic degradation from those that nonspecifically increase levels of LC3-GFP as a result of causing cellular damage or by blocking downstream lysosomal functions. The results of the screen led to the identification of eight compounds, seven of which were FDA-approved drugs, that can induce autophagy and promote long-lived protein degradation without causing obvious cellular injury (Zhang, L., Yu, J., Pan, H., Hu, P., Hao, Y., Cai, W., Zhu, H., Yu, A. D., Xie, X., Ma, D., et al. (2007). Small molecule regulators of autophagy identified by an image-based high-throughput screen. Proc Natl Acad Sci USA 104, 19023-19028).

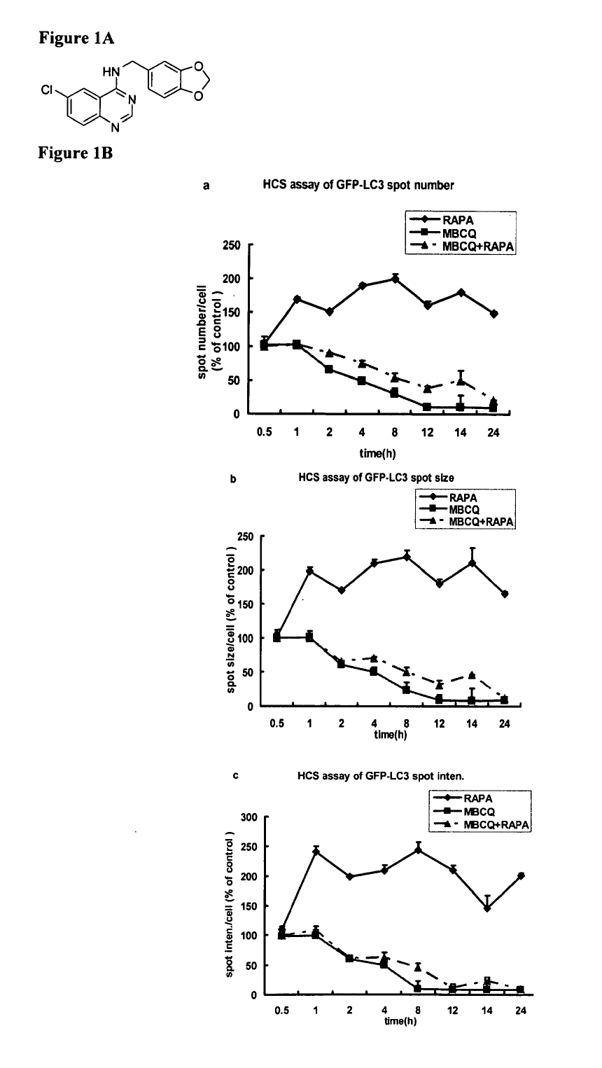

In this screen, a known bioactive compound, MBCQ (FIG. 1A), previously known as a PDE5 inhibitor (MacPherson, J. D., Gillespie, T. D., Dunkerley, H. A., Maurice, D. H., and Bennett, B. M. (2006). Inhibition of phosphodiesterase 5 selectively reverses nitrate tolerance in the venous circulation. J Pharmacol Exp Ther 317, 188-195), was identified as having autophagy inhibitor activity. Stimulation of LC3-GFP-H4 cells with rapamycin (0.2 μM) led to increases in the levels of LC3-GFP as expected. The presence of MBCQ inhibited both basal levels as well as rapamycin stimulated LC3-GFP. The reduction of LC3-GFP dots was obvious at 1 hr after the addition of MBCQ and rapamycin compared to that of rapamycin alone. Quantitative analysis of LC3-GFP dots using high throughput microscopy (FIG. 1B). The treatment of MBCQ reduced the number, spot size as well as spot intensity of LC3-GFP dots compared to the control or to rapamycin treatment alone. The intensity of LC3-GFP was measured both in the presence of both rapamycin and MBCQ together versus that of rapamycin alone, and the IC₅₀ of MBCQ was determined to be 0.788 μm, which is about 10,000 fold more potent than the commonly used type III PtdIns3P kinase inhibitor, 3-methyl-adenine (3-MA), which has the working concentration of 10 mM.

To confirm the inhibition of autophagy by MBCQ, H4-LC3 cells, 293T cells and mouse embryonic fibroblast cells were treated with MBCQ and the levels of endogenous LC3II were measured by western blot. Consistent with the inhibitory activity of MBCQ, the levels of LC3II were consistently reduced in MBCQ and rapamycin co-treated H4-LC3, 293T and MEF cells compared to that of rapamycin alone. Consistent with LC3-GFP analysis (FIG. 1B), the levels of LC3II were significantly lower after treatment with rapamycin and MBCQ for 1 h compared to that of rapamycin alone.

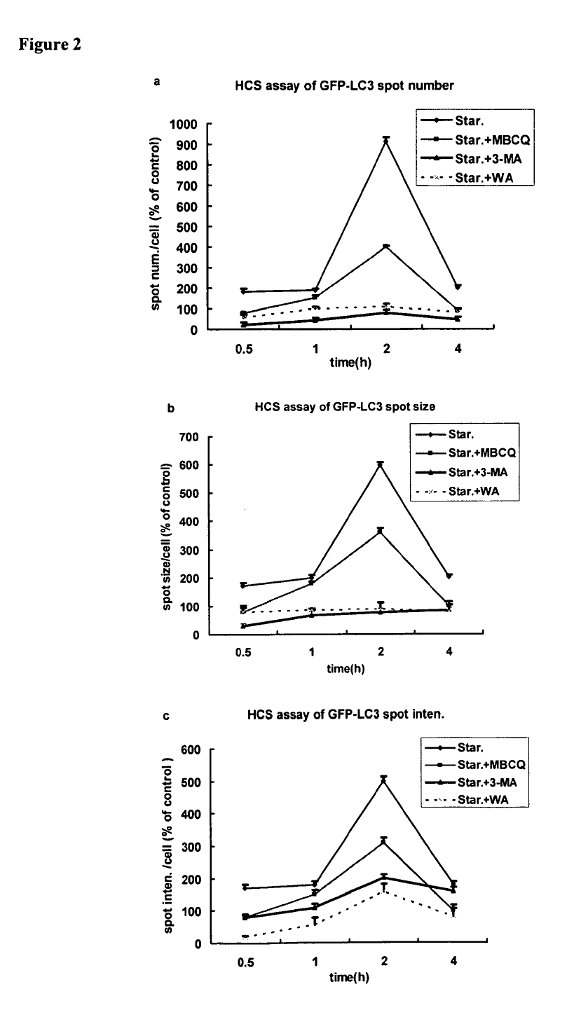

To determine the effect of MBCQ on starvation induced autophagy, H4-LC3-GFP cells were cultured in Hanks buffer for 1 h, which was sufficient to induce autophagy as demonstrated by the increases in the levels of LC3-GFP dots (FIG. 2). In the presence of MBCQ (5 μM), starvation induced autophagy is significantly reduced Quantitative measurement of the LC3-GFP spot number, spot size and spot intensity confirmed that starvation induced autophagy is inhibited by MBCQ (5 μM) or positive controls of 3-MA (10 mM) or wortmannin (0.1 μM).

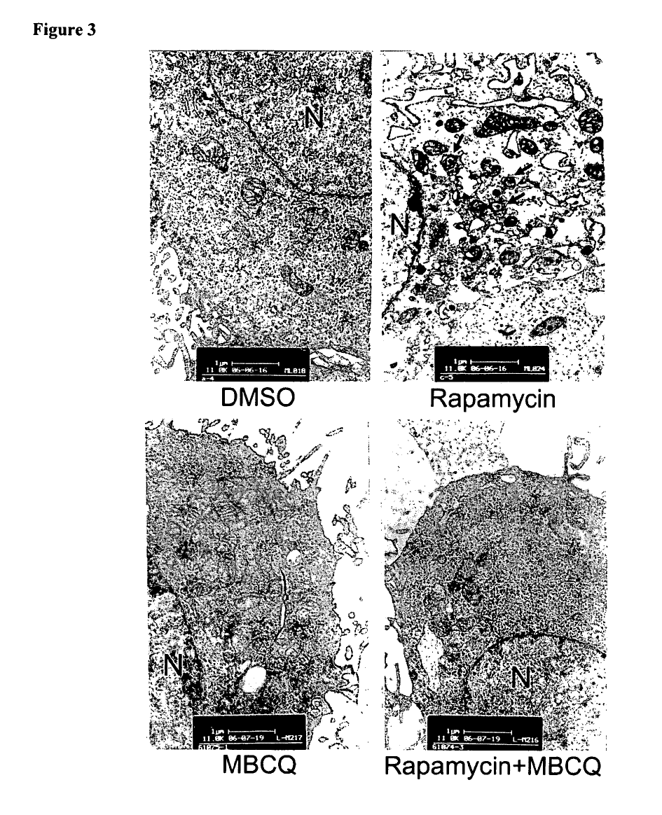

The ultra-structure of cells treated with rapamycin was determined in the presence or absence of MBCQ. It was found that the cells treated with MBCQ alone for 4 h are morphologically similar to that control treated with vehicle (1% DMSO). Treatment of rapamycin led to the formation of a large numbers of autophagosomes with characteristic double membrane. Such double membrane autophagosomes were conspicuously absent in cells treated with rapamycin and MBCQ together (FIG. 3).

### Example 2

### Structure Activity Relationship (SAR) of MBCQ

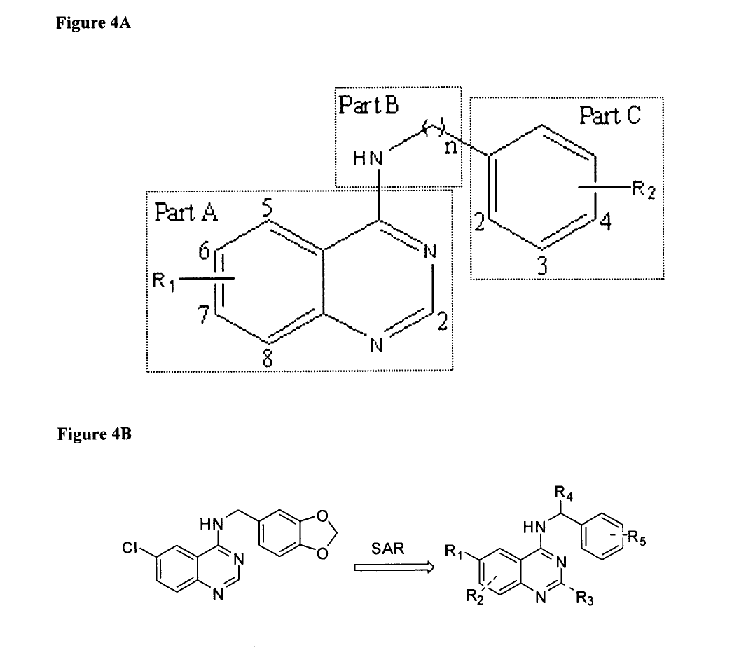

MBCQ is a 4-heteroatom-substituted quinazoline compound. For the purposes of the SAR, the structure of MBCQ was divided into three parts—parts A, B and C—as shown in FIG. 4A.

In part A, different substituents were introduced into 6-position: halogens, electron-deficient groups (e.g., nitro and methyl sulfonyl group), and electron-rich groups (e.g. methoxy and amino group); halogens were introduced into 7-position; halogens were introduced into both 6- and 8-position; and methyl or amino group were introduced into 2-position.

For part B, the nitrogen was replaced with an oxygen or sulfur atom; the methylene chain was extended; and a branch point (i.e. substitution) was added to the methylene chain.

In part C, the effects of different aromatic cycles were investigated, including: 4-pyridinyl, morpholinyl, and substituted and unsubstituted phenyl. Substituted phenyl substituents included both electron-withdrawing groups (e.g., halogen, nitro, and trifluoromethyl group) substituted phenyl 5) and electron-donating groups (e.g. amino, methoxy group).

A total of 194 compounds with above modifications on the MBCQ structure were synthesized and their activities in inhibiting autophagy were analyzed.

The SAR results can be summarized as follows (see also FIG. 4B):

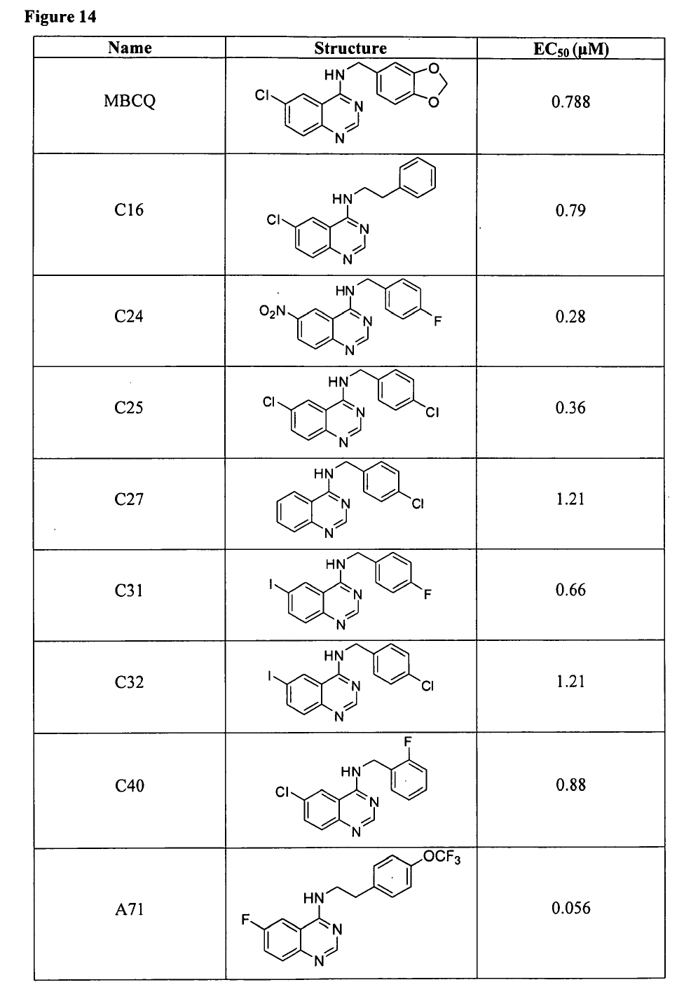

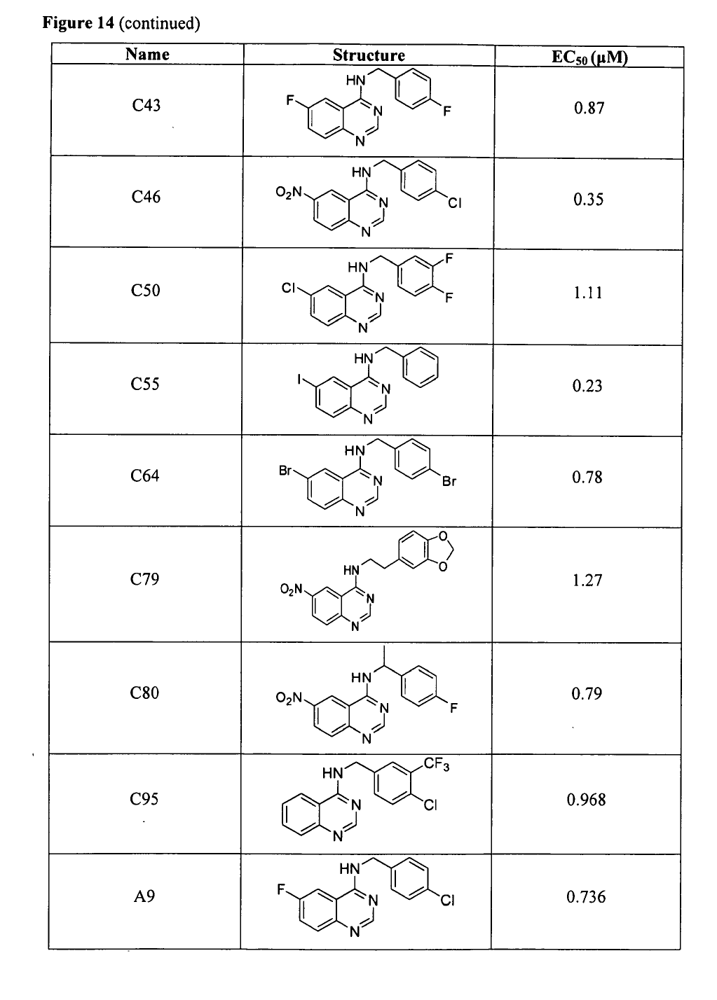

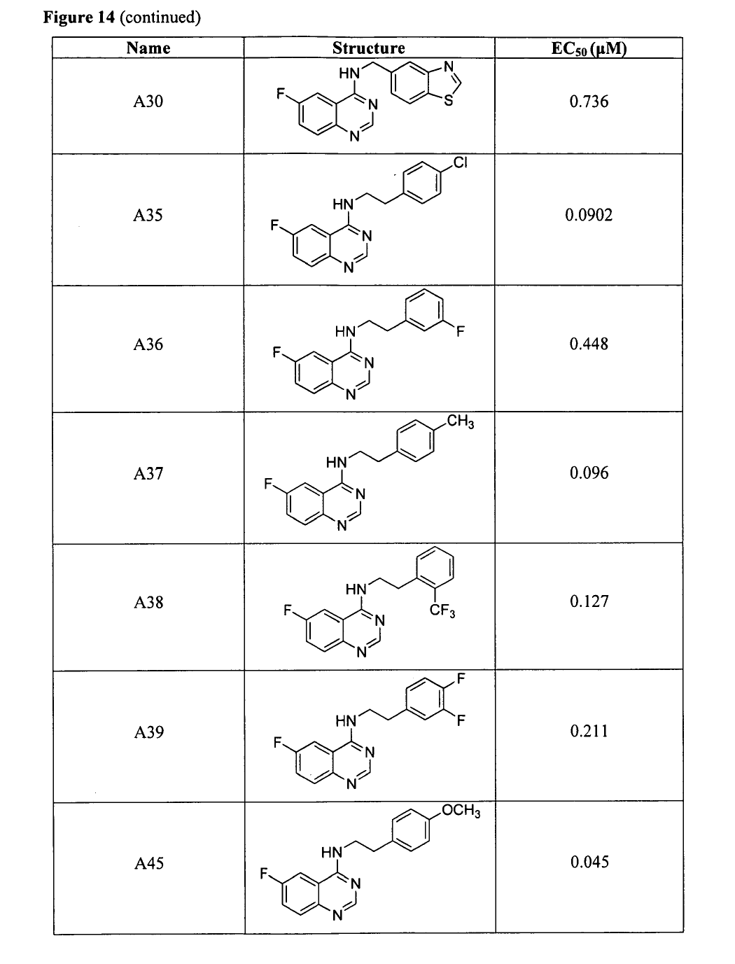

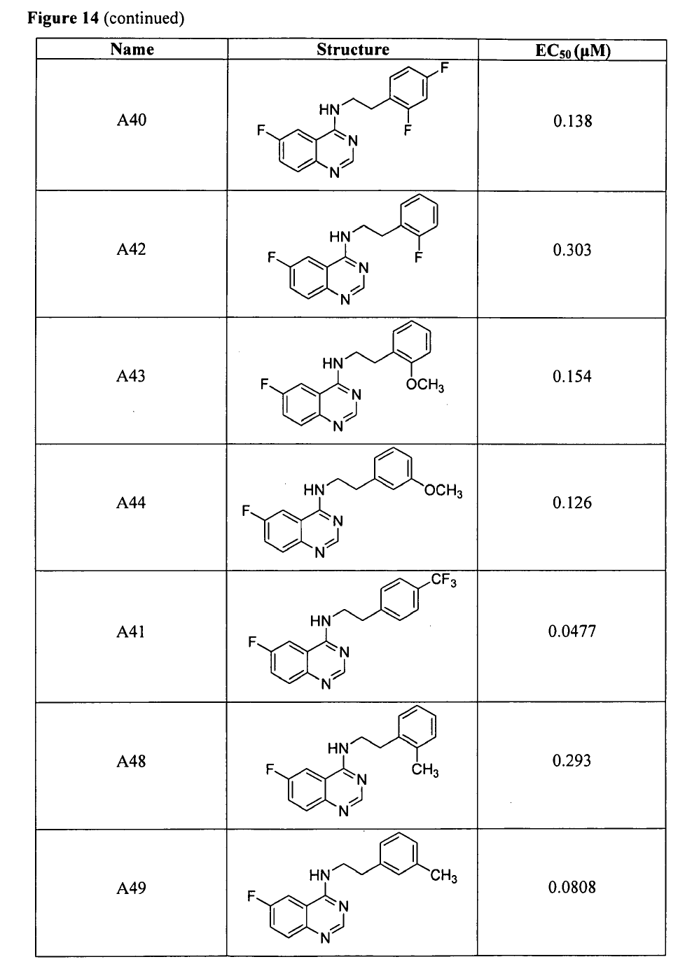

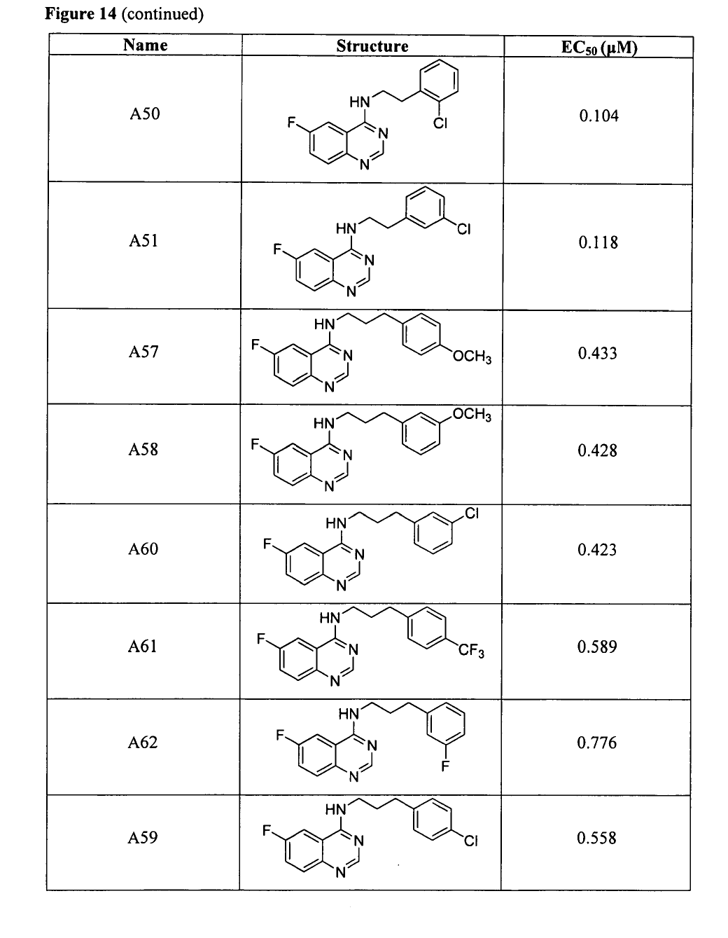

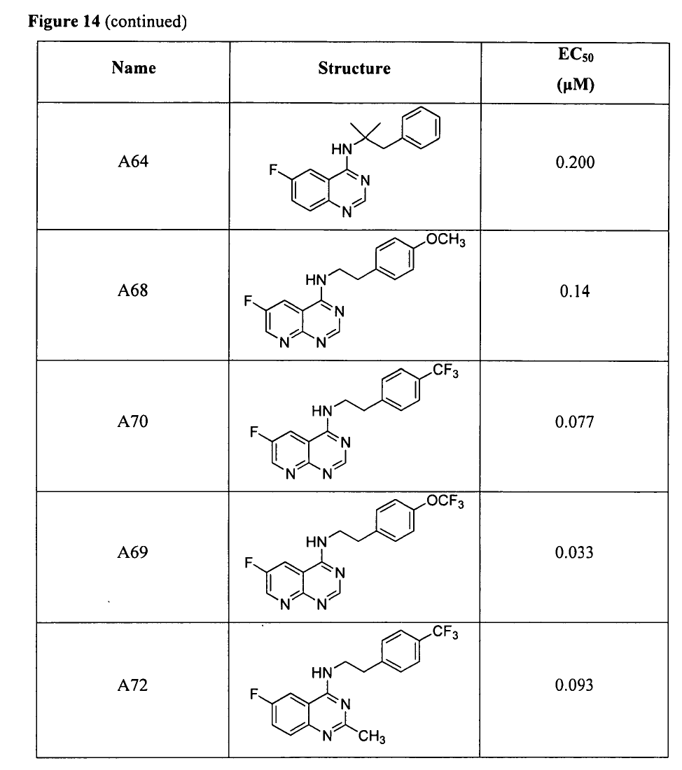

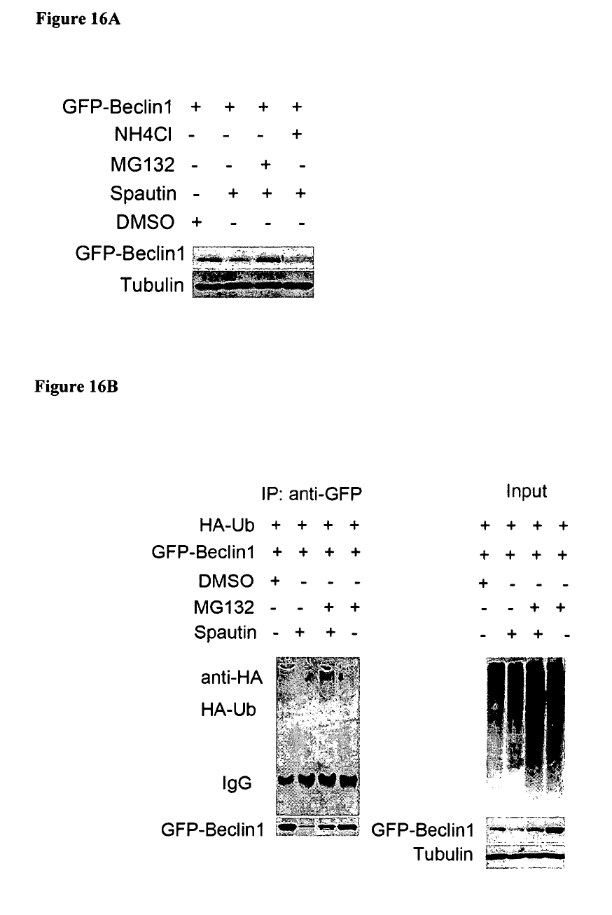

(1) The nature of substituents on 6-position of quinazoline is critical for activity. Electron-withdrawing substituents (e.g. nitro or fluorous group) improves the activity (e.g. C29 in FIG. 16). The compounds with electron-donating substituents (e.g. amino group) on 6-position has no activity (e.g. C71 in FIG. 14). Compounds without substituents on 6-position have moderate activity.

(2) Substituents on 7- and 8-position have negative effect on activity. For example, when the quinazoline is mono-substituted on 7- or 8-position, the compound loses activity (e.g. C83), and the same as compounds that are bis-substitued with chloro group both on 6- and 8-position (e.g. C19, C20).

(3) Steric hindrance on the part A impedes activity (e.g. C68, C01).

(4) When heteroatom in the part B is O or S, no activity was detected (e.g. C101, C45).

(5) Compounds lose activity when the benzene in part C is replaced with morpholine or furan (e.g. C78, C54).

(6) Compounds with 4-CF₃, 4-NO₂ or 4-pyridine in the part C exhibit no activity (e.g. C15). When there are substituents on 3-, 4- and 5-position simultaneously, no activities were detected (e.g. C15).

(7) High activity was observed when heteroatom in the part B was nitrogen, which linked with 1-3 carbons (e.g. C16, C51 and C13). No activity was detected when more than three methylene units are in the chain linking part A and part C (e.g. C30, C49). In addition, bulky substituents on the branch chain leads to no activity (e.g. C81, C86 and C94). Further, no appreciable effect on activity was detected with different optical configuration (R or S) on branch chain (e.g. C69 and C84, C76 and C77).

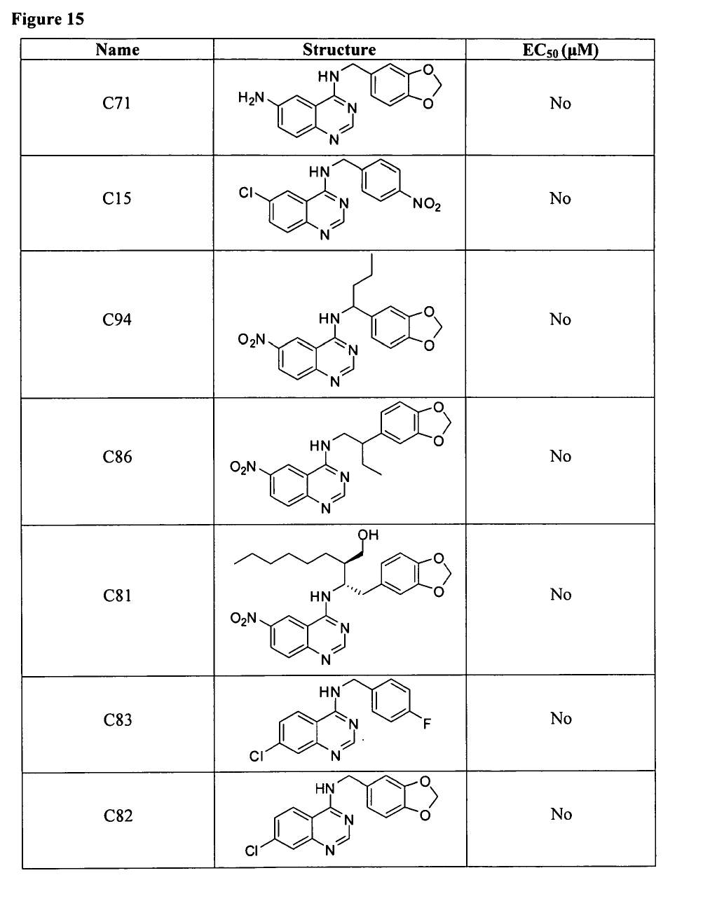

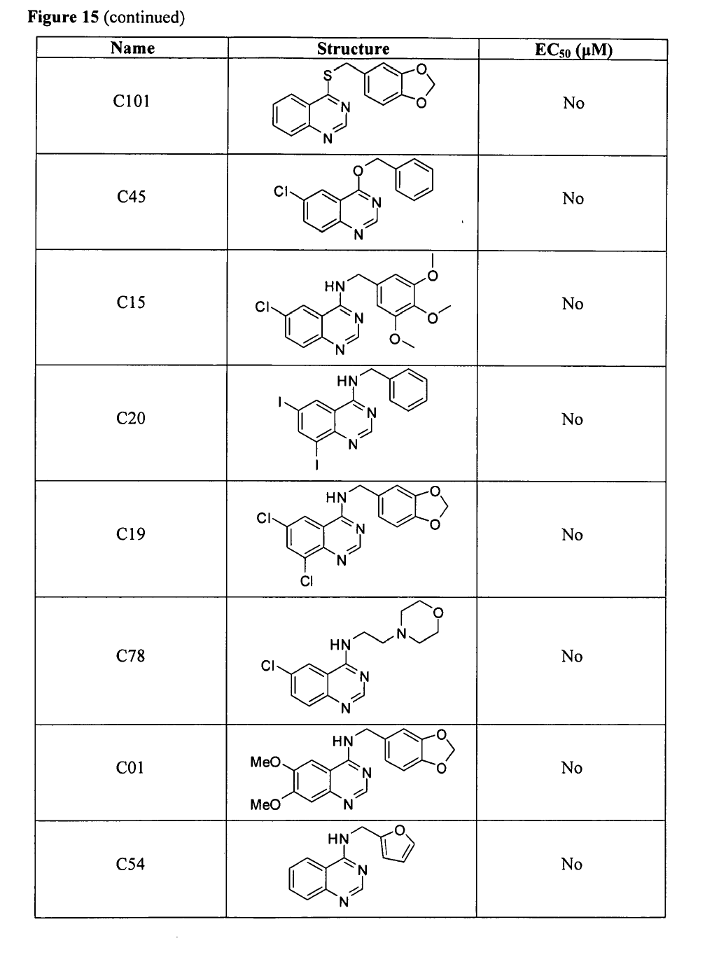

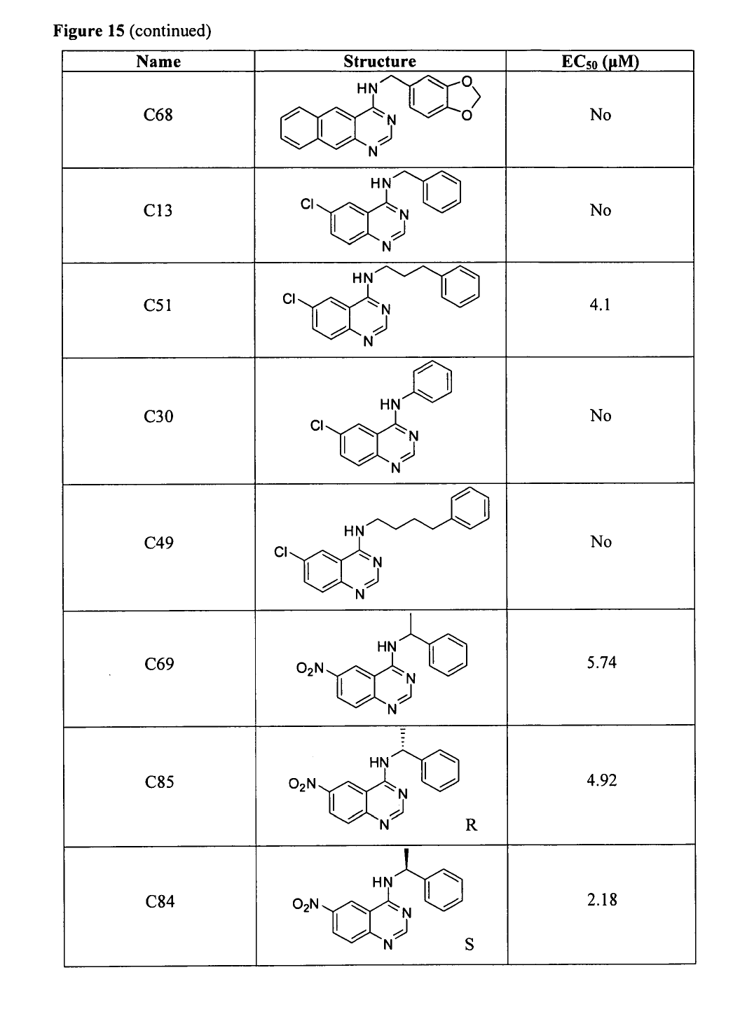

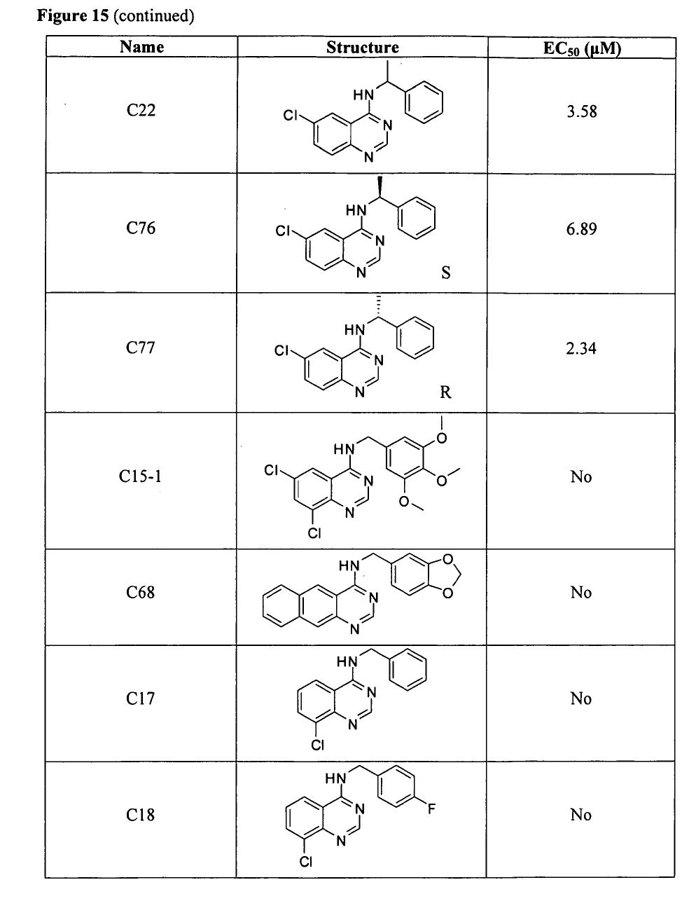

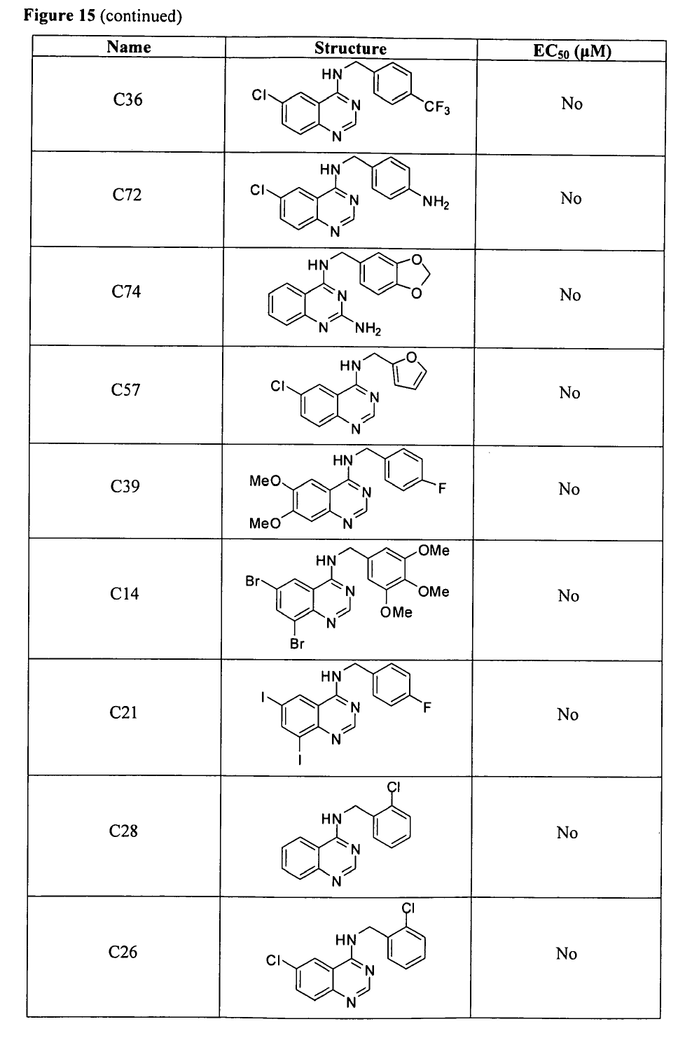

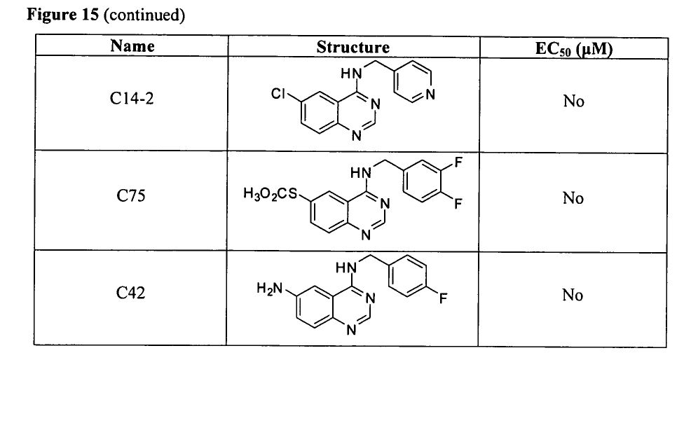

Among the MBCQ derivatives synthesized and analyzed for their autophagy inhibiting activity, 44 compounds exhibited autophagy inhibitory activity similar or above that of MBCQ (FIG. 16). At the same time, a number of compounds were identified, such as C71 and C82, which are similar to MBCQ structurally but have no autophagy inhibitory activity and were used as negative controls in subsequent experiments (FIG. 15).

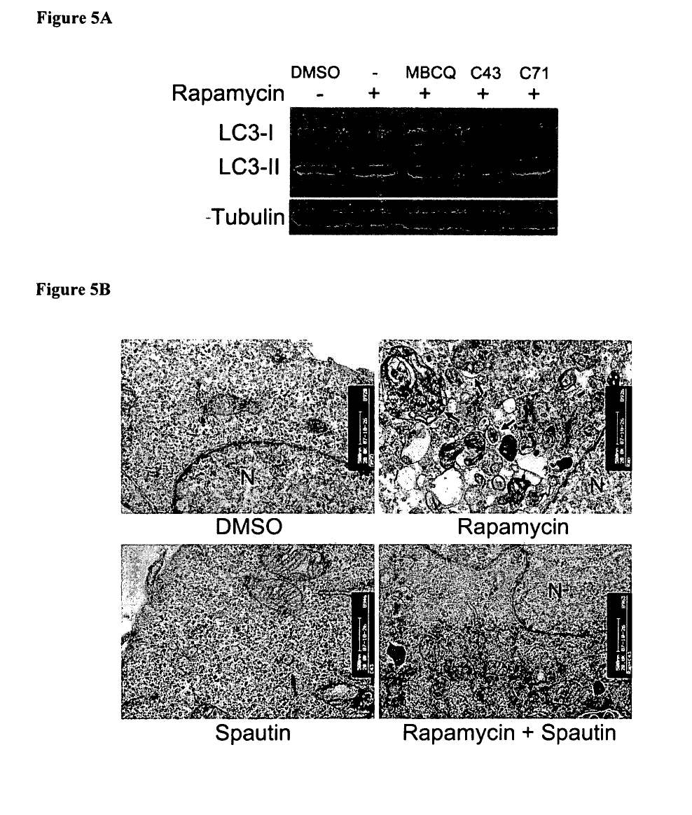

To confirm the inhibitory activity on autophagy, mouse embryo fibroblasts (MEF) cells were treated with C29, C43 or C71 for 4 hours in the presence or absence of rapamycin and the levels of autophagy were determined by LC3 western blotting. The treatment of C43 or C29, but not the negative control C71, inhibited autophagy induced by rapamycin. (FIG. 5A).

The effect of C29 and C43 on autophagy was further confirmed by electron microscopy. In rapamycin treated MEF cells, numerous autophagosome vesicles with double membranes were observed, as well as many vesicles with multi-membrane as expected (FIG. 5B). In cells treated with rapamycin and C29 or C43, autophagosomes are largely absent as that is in vehicle treated cells (FIG. 5).

### Example 3

### MBCQ Inhibits Selective Cell Death Models Involving Autophagy

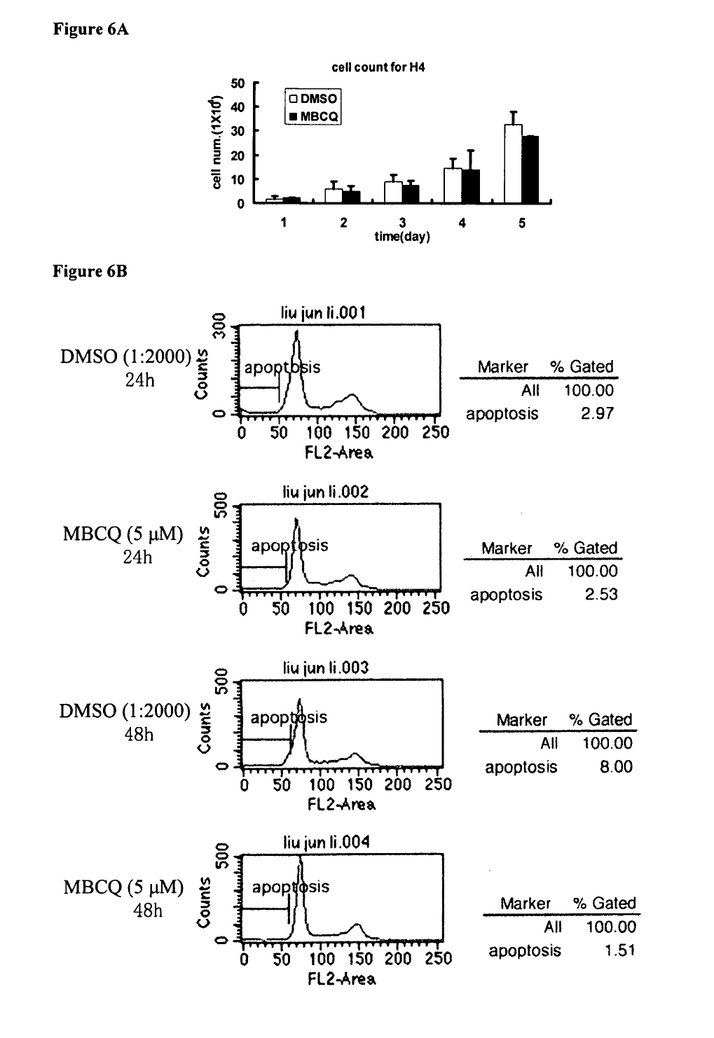

To characterize the effect of MBCQ on cellular activity, the effect of MBCQ on cell survival and cell cycle was determined as outlined below. H4 cells were treated with MBCQ (5 μM) for 5 days and harvested daily for cell number counting in the presence of trypan blue. As shown in FIG. 6A, the treatment of MBCQ had no effect on cell proliferation. The cell cycle profile and possible apoptotic cells in H4 cells treated with MBCQ (5 μM) for 24 h and 48 h was also determined. As shown in FIG. 6B, MBCQ has no detectable effect on cell cycle distribution.

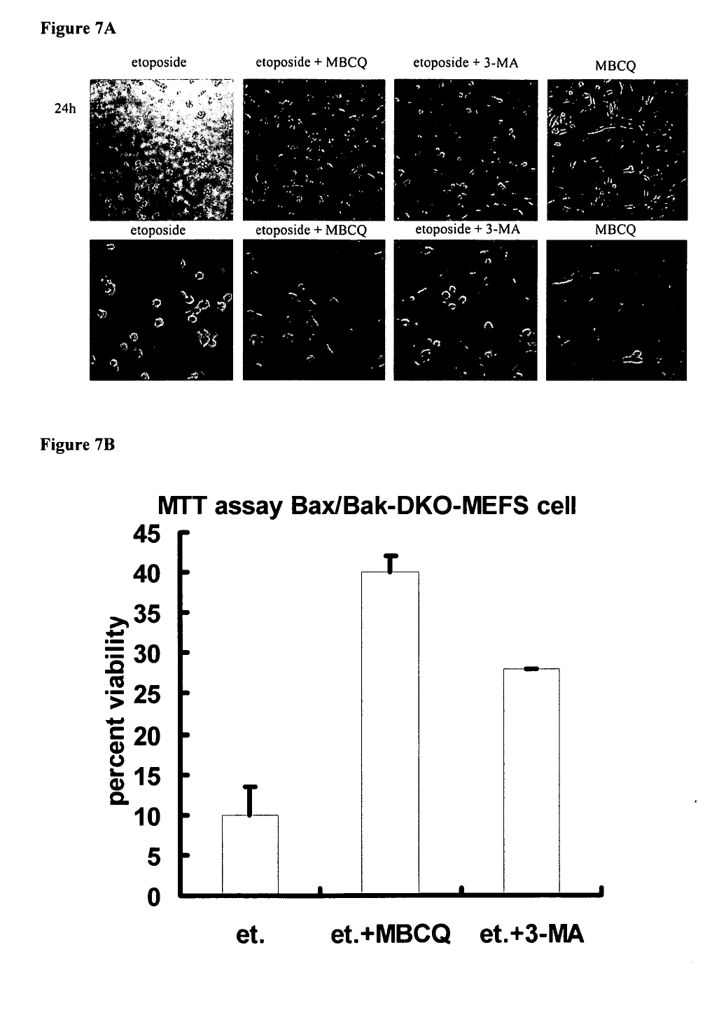

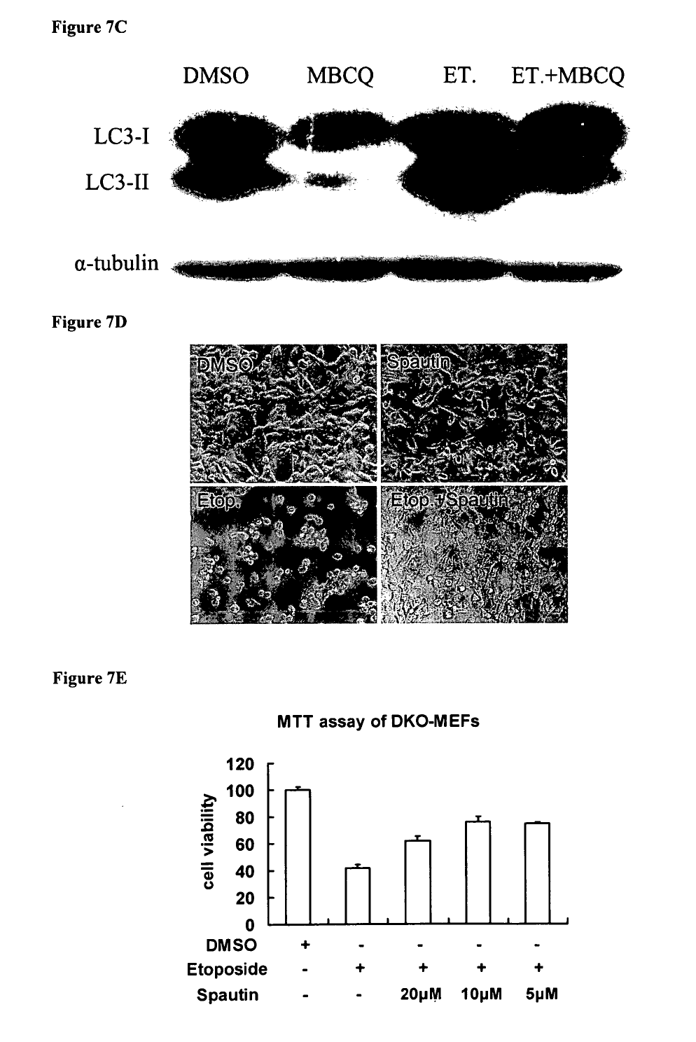

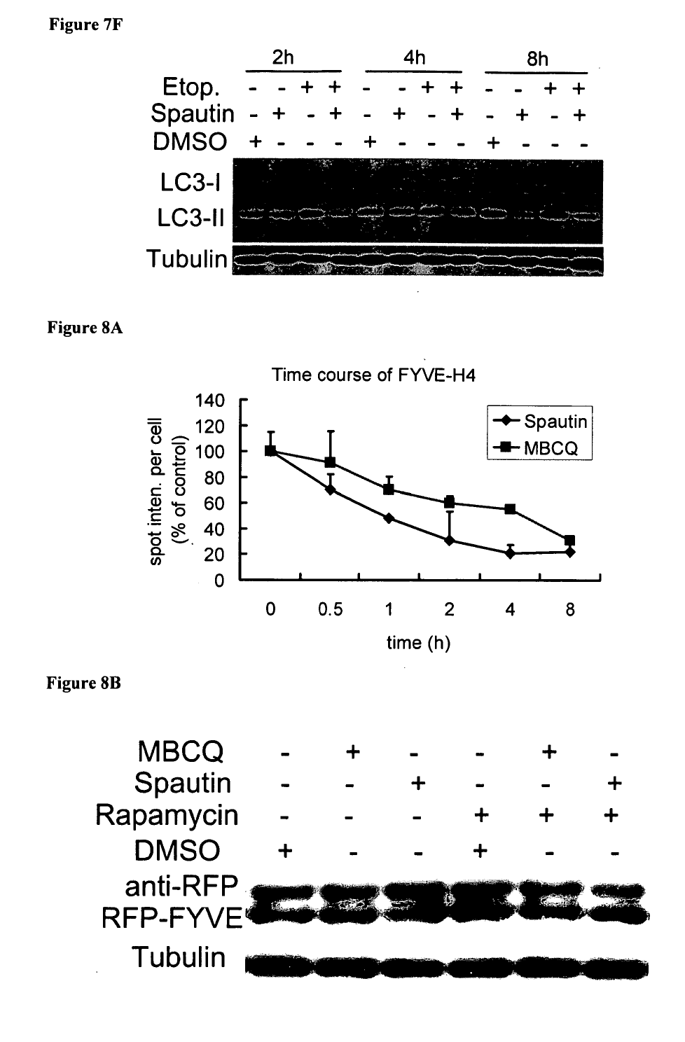

Autophagy has been proposed to contribute to cell death in a number of apoptotic deficient cell types. For example, bax/bak double deficient mouse embryonic fibroblast cells (DKO mefs) are highly resistant to apoptosis (Wei, M. C., Zong, W. X., Cheng, E. H., Lindsten, T., Panoutsakopoulou, V., Ross, A. J., Roth, K. A., MacGregor, G. R., Thompson, C. B., and Korsmeyer, S. J. (2001). Proapoptotic BAX and BAK: a requisite gateway to mitochondrial dysfunction and death. Science 292, 727-730). Stimulation of bax/bak DKO mefs with etoposide has been shown to induce cell death in part through autophagy induction (Shimizu, S., Kanaseki, T., Mizushima, N., Mizuta, T., Arakawa-Kobayashi, S., Thompson, C. B., and Tsujimoto, Y. (2004). Role of Bcl-2 family proteins in a non-apoptotic programmed cell death dependent on autophagy genes. Nat Cell Biol 6, 1221-1228). To test if MBCQ may inhibit cell death of bax/bak DKO cells induced by etoposide, Bax/bak DKO cells were treated with etoposide in the presence of MBCQ (10 μM), or 3-MA (10 mM) as a positive control for 8 h. As shown in FIG. 7A, the presence of MBCQ significantly reduced cell death of bax/bak DKO MEF cells. Furthermore, consistent with inhibition by MBCQ, the levels of LC3II were increased in etoposide treated cells but reduced in the presence of MBCQ (FIG. 7B).

### Example 4

### MBCQ Selectively Reduces the Cellular Levels of PI3P

Since MBCQ inhibits autophagy induced by rapamycin and starvation, it was first determined if MBCQ affects the activity of mTOR. Western blotting assays demonstrated that MBCQ has no effect on the phosphorylation of mTOR and its targets, p70S6K and S6, in control or rapamycin treated cells. Nor does MBCQ have any effect on the phosphorylation of GSK-3α/β, AKT. Since the phosphorylation of AKT is regulated by type I PtdIns3(PI3) kinase, this result also suggests that MBCQ has no effect on type I PI3 kinase. Thus, it was concluded that MBCQ has no effect for the mTOR pathway or type I PI3 kinase.

The effects of MBCQ on early endosomes using immunostaining of EEA1 as a marker, lysosomes using immunostaining of lamp2 as a marker or lysotracker, trans-Golgi using GalT-YFP as a marker was determined. No effect of MBCQ was detected in any of these experiments. Thus, it was concluded that MBCQ does not affect major intracellular organelles.

In addition, the effect of MBCQ on proteasomal degradation pathway using pEGFP-CL1, a GFP fusion with a short-lived peptide was determined (Bence, N. F., Sampat, R. M., and Kopito, R. R. (2001). Impairment of the ubiquitin-proteasome system by protein aggregation. Science 292, 1552-1555). It was found that MBCQ does not affect the levels of pEGFP-CL1, suggesting that MBCQ does not have a general effect on the proteasomal pathway (data not shown). In addition, the treatment of MBCQ has no effect on the general levels of polyubiquitination. Thus, it was concluded that MBCQ does not have a general effect the ubiquitin-proteasomal degradation pathway.

The levels of PtdIns3P (PI3P) are known to play a critical role in mediating autophagy (Levine, B., and Klionsky, D. J. (2004). Development by self-digestion: molecular mechanisms and biological functions of autophagy. Dev Cell 6, 463-477). To ask if MBCQ has an effect on PI3P, H4 cells expressing FYVE-RFP were used. FYVE binds specifically to PI3P and is widely used as a marker for cellular levels for PI3P (Gaullier, J. M., Simonsen, A., D'Arrigo, A., Bremnes, B., Stenmark, H., and Aasland, R. (1998). FYVE fingers bind PtdIns(3)P. Nature 394, 432-433). Interestingly, the treatment of MBCQ rapidly and effectively reduced the levels of FYVE-RFP spots in both basal and rapamycin treated H4 cells while the levels of FYVE-RFP detected by western blotting were not changed (FIG. 8).

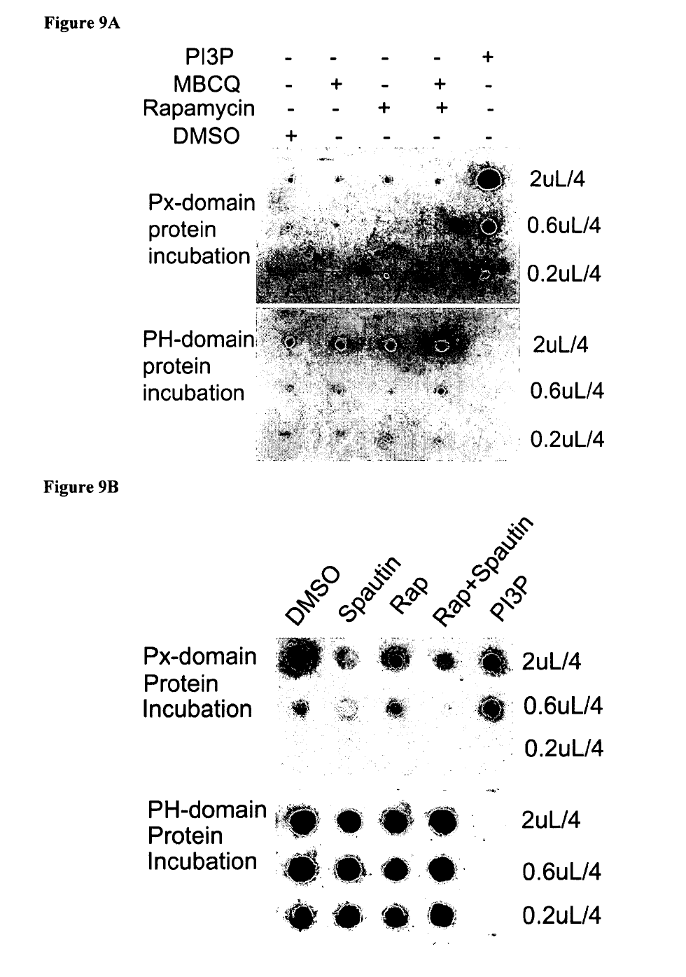

To further determine the effect of MBCQ on the cellular levels of PtdIns3P, a lipid dot blot assay was used. The cellular PtdIns species were extracted and applied onto polyvinylidene fluoride membrane. The levels of PtdIns3P was detected using GST-PX domain protein and anti-GST antibody. As shown in FIG. 9, the treatment of MBCQ and C43 selectively reduced the cellular levels of PtdIns3P in both basal and rapamycin treated cells. Taken together, it was concluded that MBCQ reduces the levels of PtdIns3P.

### Example 5

### MBCQ and its Active Derivatives Selectively Promotes the Degradation of Vps34 Complexes

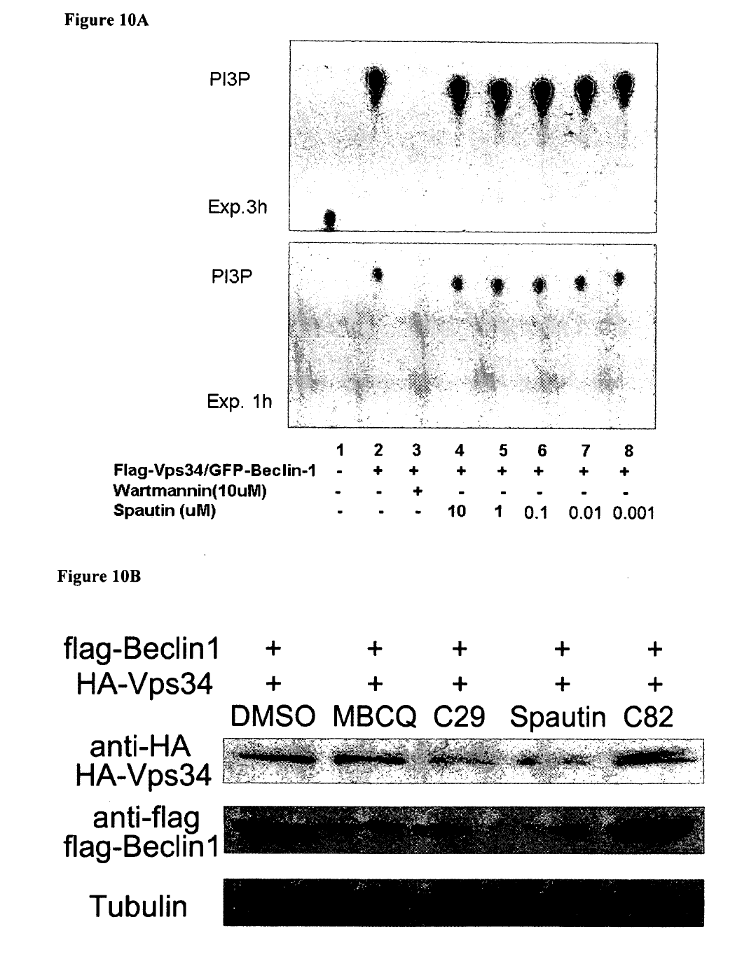

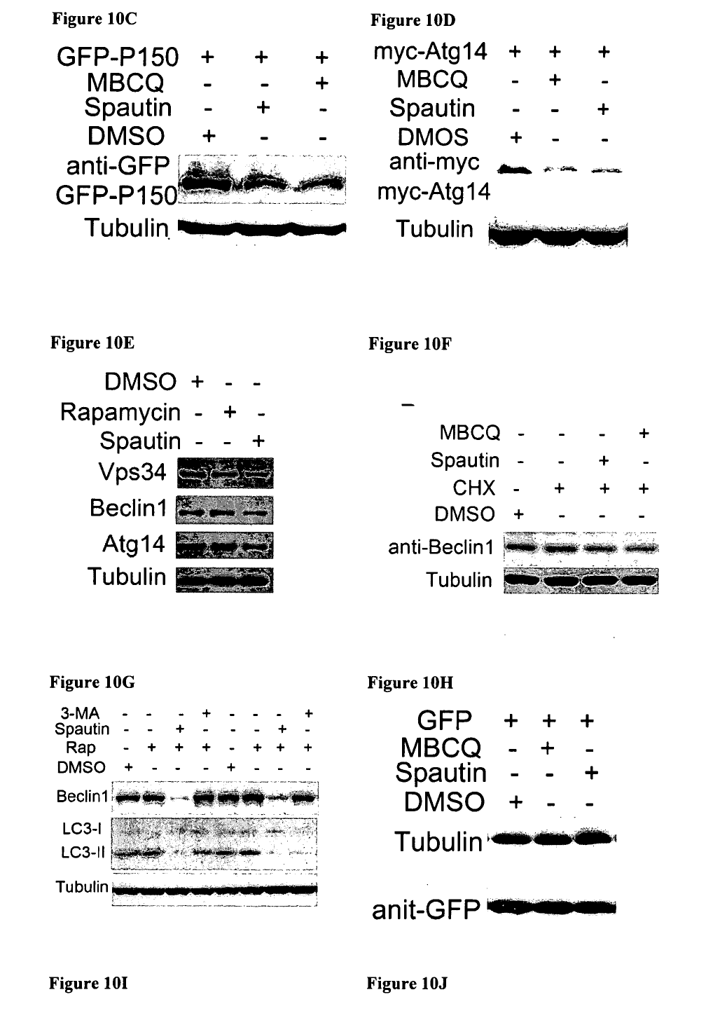

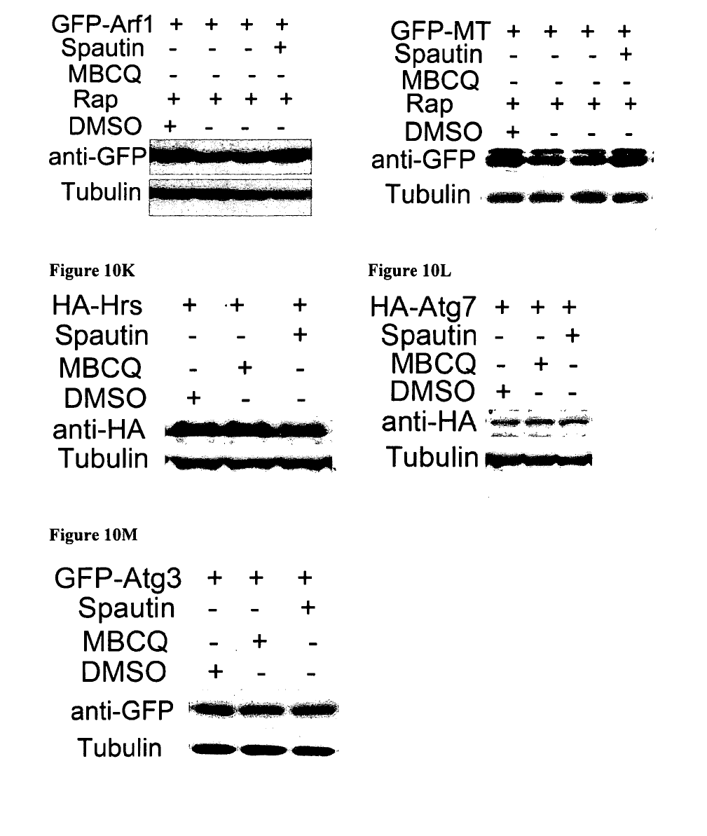

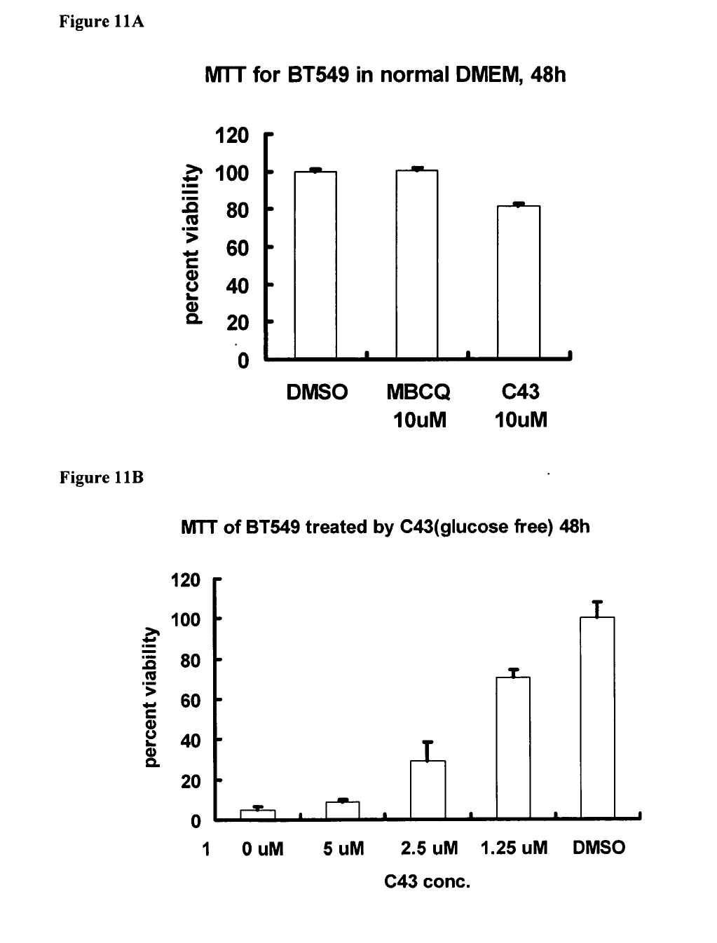

Since the type III PtdIns3 kinase complex, Vps34/Beclin1/p150, is responsible for the phosphorylation of PtdIns to produce PtdIns3P, MBCQ inhibitory activity on the kinase activity of the Vps34 complex was determined. 293T cells were transfected with HA-Vps34/GFP-Beclin1. The Vps34 complex immunoprecipitated using anti-HA was incubated with PtdIns in the presence of γ-32P-ATP. The phosphorylation product was analyzed by thin layer chromatography and followed by autoradiography. As shown in FIG. 10A, the phosphorylation of PtdIns is inhibited by wortmannin but not by MBCQ. Thus, it was concluded that MBCQ is not a direct inhibitor of Vps34 enzymatic activity.

On the other hand, it was noted that the levels of flag-tagged Beclin1 and HA-Vps34 were considerably lower in MBCQ, C29 or C43 treated cells than that of C82, an inactive analog (FIG. 10B). The treatment of MBCQ, C29 and C43, but not C82, also reduced the levels of GFP-p150 and Atg14L (FIGS. 10C-D).

It was also found MBCQ and C43 could reduce the levels of endogenous Beclin1, Vps34 and Atg14L (FIG. 10E) in H4 cells and in 293T cells (FIG. 10F), while the known autophagy inhibitor 3-MA has no effect on endogenous Beclin1 in H4 cells (FIG. 10G).

To determine if MBCQ and C43 have similar effects on endogenous Beclin1, 293T cells were treated with MBCQ or C43 in the presence of CHX to inhibit protein synthesis. The levels of Beclin1 were notably lower in the presence of MBCQ or C43 than with CHX alone after treatment for 6 h (FIG. 10E). Thus, it can be concluded that both MBCQ and C43 may promote the degradation of endogenous Beclin1.

To explore the mechanism by which MBCQ and C43 reduce the levels of Vsp34 complexes, 293T cells were treated with C43 with proteasomal inhibitor MG132 or NH₄Cl to inhibit lysosomal degradation. It was found that the presence of MG132 but not NH₄Cl inhibited the reduction of GFP-Beclin1. This result suggests that the treatment of C43 promotes the degradation of Beclin1 through the proteasomal pathway. It was therefore concluded that C43 inhibits autophagy by selectively promoting the degradation of type III PI3 kinase complexes including Vps34/Beclin1/p150/Atg14L/UVRAG.

### Example 6

### MBCQ and its Active Derivatives Enhances Starvation-Induced Apoptosis

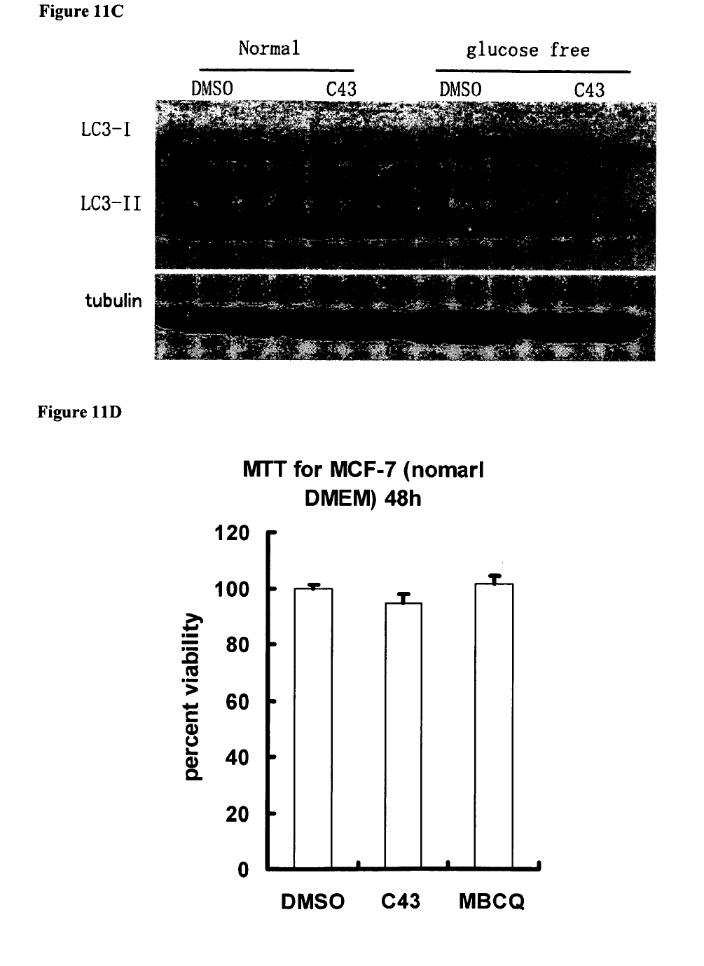

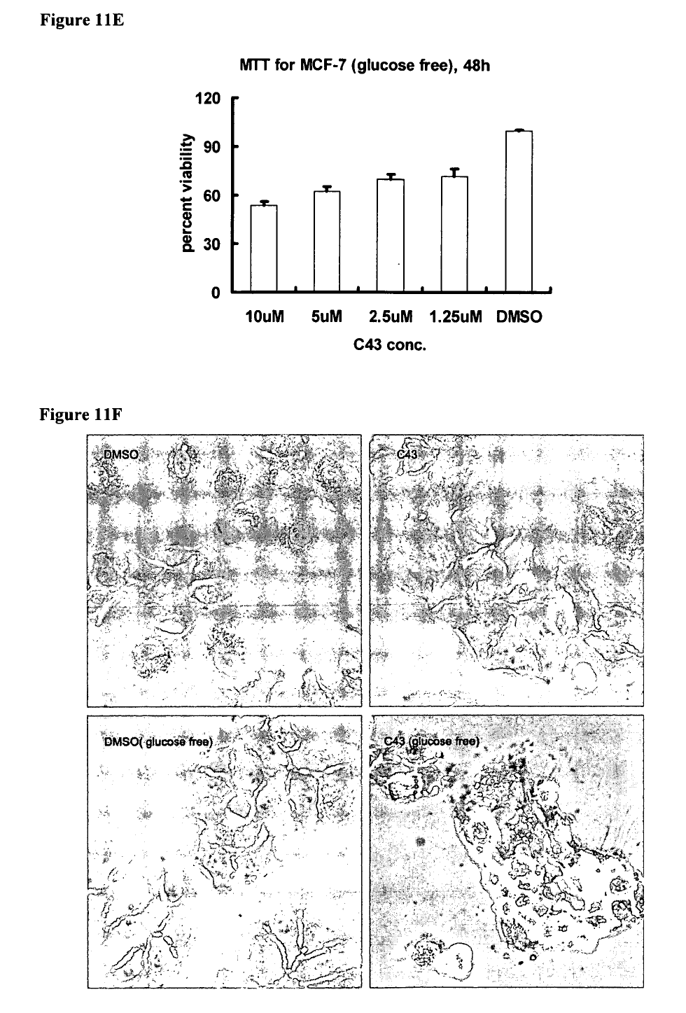

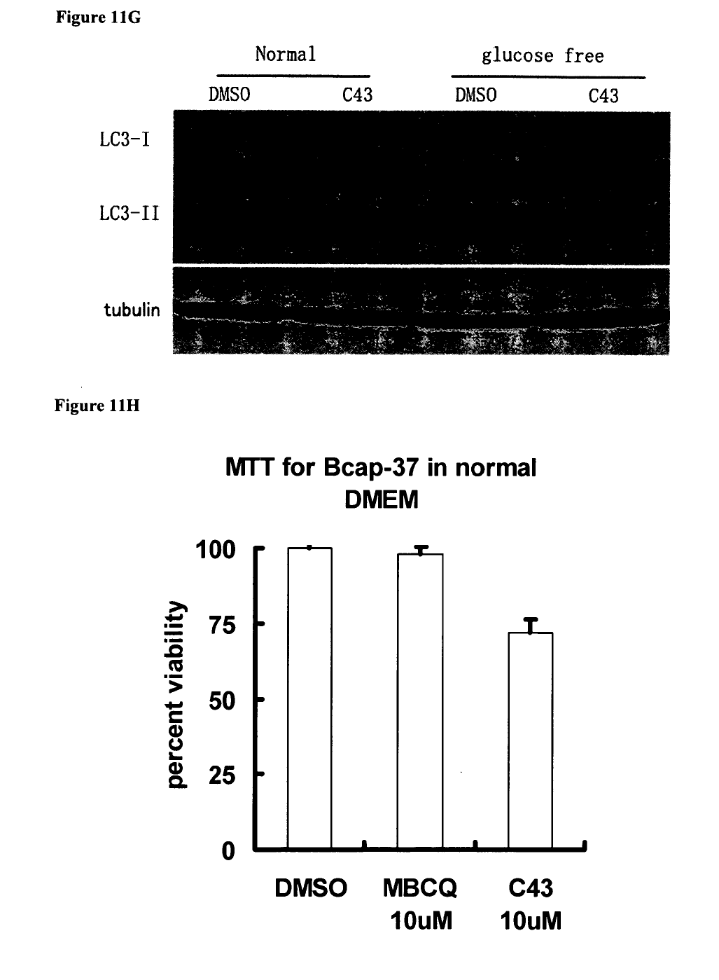

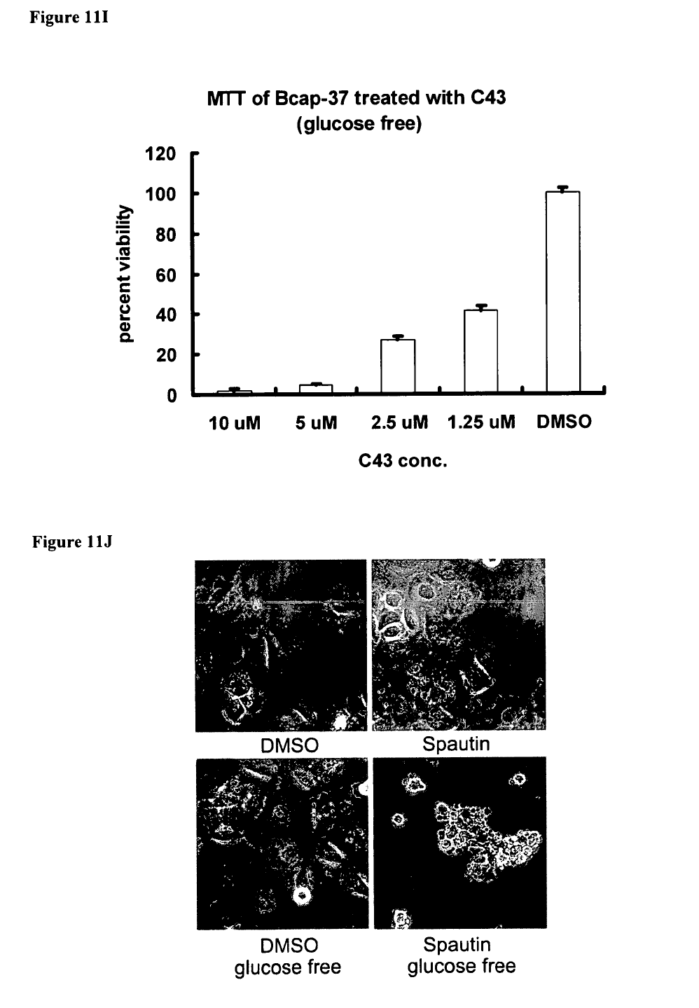

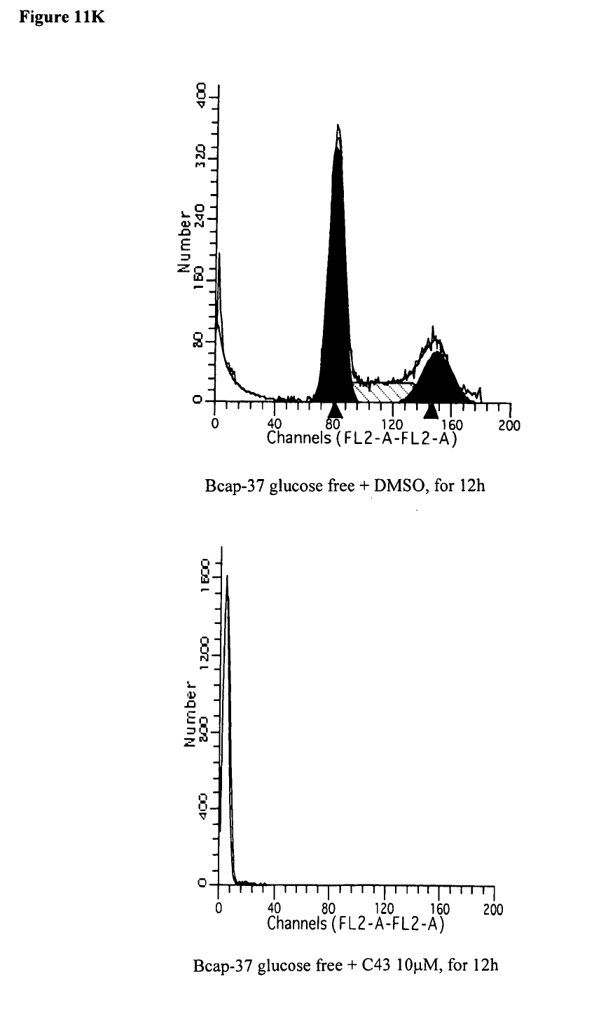

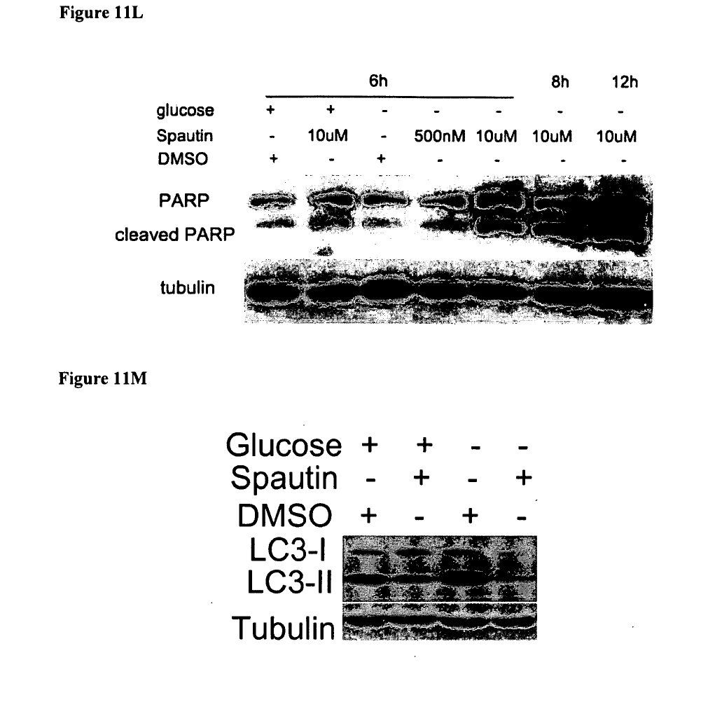

Since autophagy is activated under metabolic stress conditions to support cell survival, compounds were tested to determine if they promote cell death under starvation condition. Indeed, it was found that C43 reduced the survival of MDA-MB-231 cells under serum free condition (FIG. 11A) and MCF-7 cells under glucose-free condition (FIG. 11B). Western blot analysis confirmed that the treatment of C43 inhibited autophagy in MCF-7 cells under both basal and glucose-free condition.

In addition, it was found that C43 inhibited the proliferation of Bcap-37 cells, a breast cancer cell line, in the presence of 10% bovine serum (FIG. 11C). Further, Mcap-37 cells became highly sensitive to C43 under glucose free condition (FIG. 11D). Western blot analysis of Bcap-37 cells cultured under control and glucose-free condition confirmed that the treatment of C43 inhibited autophagy under both basal and glucose-free conditions.

To explore the mechanism by which C43 induces the death of Bcap-37 cells, the DNA content was analyzed by FACS. It was found that the treatment of Bcap-37 cells under glucose-free condition induced a peak of sub-diploid DNA, consistent with apoptotic DNA fragmentation (FIG. 11E). Furthermore, cleavage of PARP, a hallmark of caspase activation, was also detected in Bcap-37 cells treated with C43 under glucose-free condition for 6 h (FIG. 11F). Another breast cancer cell line, BT549, also demonstrated a similar response towards the treatment of C43.

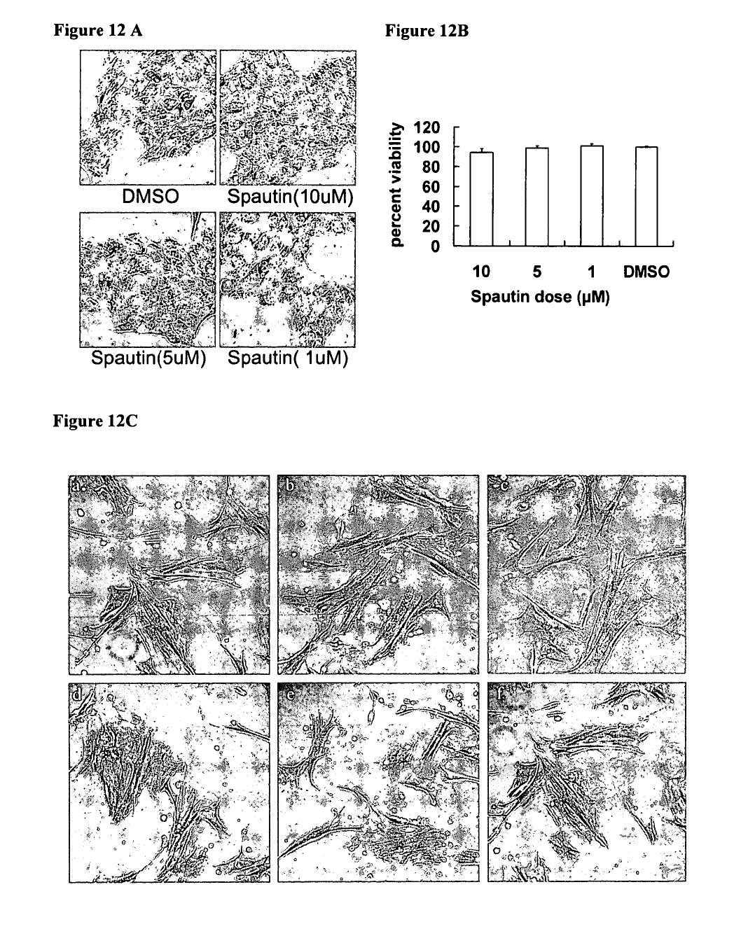

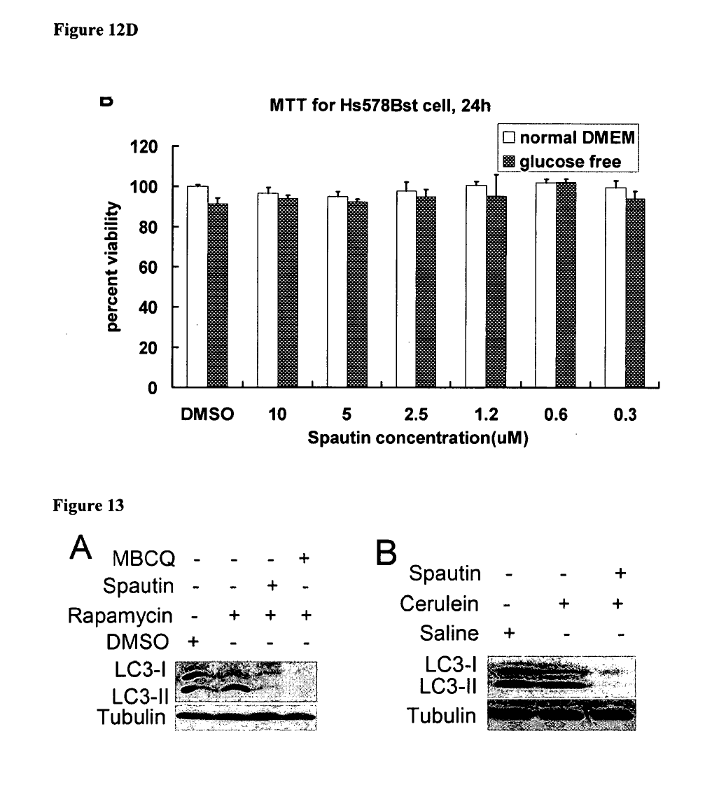

In contrast to the above cancer cell lines analyzed, the treatment of MDCK cells, which derived from the Madin-Darby canine kidney, with spautin under glucose-free condition did not induce apoptosis; only ˜25% growth suppression was observed when treated with 10 μM of spautin for 48 hrs (FIGS. 12A and 12B). Hs578Bst cells, established from normal tissue peripheral to the tumor and is myoepithelial in origin, also were not sensitive to spautin (FIGS. 12C and 12D). These results are consistent with the possibility that cancer cells may be under increased metabolic pressure and therefore more sensitive to the inhibition of autophagy than non-cancer cells.

Increased activation of autophagy under apoptotic deficient conditions has been shown to mediate cell death. To test this possibility, Bax-Bak double knockout (DKO) cells were tested with etoposide to induce by DNA damage response in the presence or absence of spautin and it was found that C43, MBCQ and 3-MA inhibits etoposide induced death of Bax-Bak DKO cell

Thus, it was concluded that a subset of cancer cells may be selectively sensitive to inhibition of autophagy.

### Example 7

### Effect of MBCQ Derivatives in Vivo

To begin to test the effect of MBCQ derivatives in vivo, the ability of MBCQ derivatives to inhibit autophagy in rapamycin injected mice was investigated. Mice were injected with rapamycin (10 mg/kg) alone as a positive control, or with C43 or MBCQ (40 mg/kg) intraperitoneally every hour for 4 h and then sacrificed at the fifth hour. The autophagy levels in liver were then analyzed by western blotting using anti-LC3 antibody. As shown in FIG. 13A, administration of C43 or MBCQ significantly reduced the levels of LC3II. Thus, it was determined that C43 and MBCQ are both active in vivo in inhibiting autophagy.

Since autophagy has been proposed to contribute to the tissue damage in pancreatitis, MBCQ derivatives were tested to see if they could reduce tissue damage induced by cerulein injection, a well-established animal model of pancreatitis (Hashimoto, D., Ohmuraya, M., Hirota, M., Yamamoto, A., Suyama, K., Ida, S., Okumura, Y., Takahashi, E., Kido, H., Araki, K., et al. (2008). Involvement of autophagy in trypsinogen activation within the pancreatic acinar cells. J Cell Biol 181, 1065-1072; and Ohmuraya, M., and Yamamura, K. (2008). Autophagy and acute pancreatitis: a novel autophagy theory for trypsinogen activation. Autophagy 4, 1060-1062). Rats were injected intraperitoneally with cerulein (50 ng/kg) alone or with C43 (40 mg/kg) hourly for 4 times. The rats were sacrificed at one hr after the last injection and the pancreas were isolated for western blotting analysis. As shown in FIG. 13B, the injection of cerulein induced autophagy as reported; the co-injection of C43 significantly reduced the levels of autophagy induced by cerulein injection. Taken together, it was concluded that C43 is effective in reducing autophagy induced in cerulein induce pancreatitis.

### Example 8

### Preparation of Compounds

One general approach to the synthesis of compounds of formula I and II is depicted below in Scheme 1.

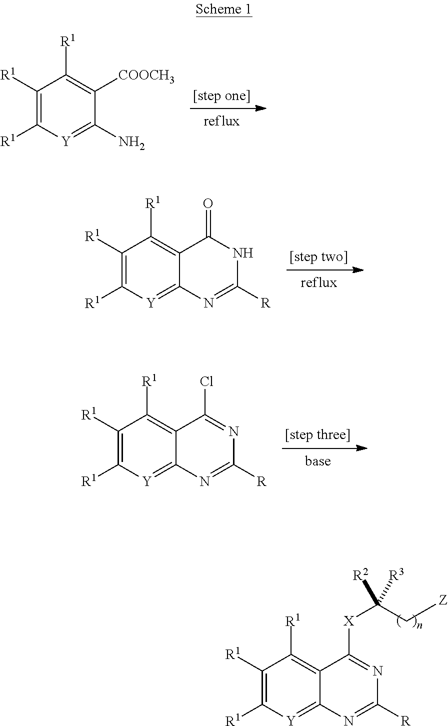

[1] Step one is the formation of a quinazoline-4-ketone (or 8-aza-quinazoline-4-ketone).

In one approach, anthranilic acid methyl ester (or methyl 2-aminonicotinate) is mixed with formamide in a molar ratio of 1:15-20 and heated at about 170-190° C. After the reaction is complete, the mixture is cooled, leached, washed and dried. The resulting crude product is used in the next reaction without further processing.

[2] Step two is the formation of a 4-chloroquinazoline (or 8-aza-4-chloroquinazoline).

In one approach, the crude product from step one is mixed with phosphorus oxychloride in a molar ratio of 1:8.7-10, then heated at about 100-115° C. After the reaction is complete, approximately 10-12 hours, the mixture is cooled and excess phosphorus oxychloride is removed by rotary evaporation. An organic solvent, such as dichloromethane, is added to dissolve the solid, followed by pH adjustment of the resulting solution to about 7-8 by addition of ammonia. The resulting mixture is extracted with dichloromethane, dried and purified by column chromatography.

In another approach, the crude product from step one is mixed with thionyl dichloride in a molar ration of 1:15-20, with catalytic amount of anhydrous DMF (e.g. 0.5-1 mL), then heated at about 80-90° C. After the reaction is complete, approximately 10-12 hours, the mixture is cooled and excessive thionyl dichloride was removed by rotary evaporator. An organic solvent, such as dichloromethane, is added to dissolve the solid, followed by pH adjustment of the resulting solution to about 7-8 by addition of ammonia. The resulting mixture is extracted with dichloromethane, dried and purified by column chromatography.

In another approach, the crude product from step one is mixed with oxalyl chloride under argon and anhydrous DMF is added dropwise, to form a mixture with a molar ratio of 1:1.5:1.5 product of step one:oxalyl chloride:DMF, and then heated to about 85-95° C. After about 7-10 hours the reaction is quenched with saturated disodium hydrogen phosphate. Then the reaction mixture is then extracted with an organic solvent, such as dichloromethane, by column chromatography.

[3] Step three is the formation of an N-substituted-4-amino-quinazoline (or 8-aza-N-substituted-4-amino-quinazoline).

Under argon, the product of step 2, HXC(R²)(R³)(CH₂)ₙZ (as defined herein), and triethylamine are combined in a molar ratio of 1:1.25:1.68, in an organic solvent, such as tetrahydrofuran, and heated to about 75-80° C. After about 12-18 hours, the organic solvent is removed by rotary evaporation. The resulting crude product is purified by column chromatograpy.

For additional illustration, the synthesis of compound A9, A30 and A36 are described in more detail below. As noted above, additional compounds can be prepared by varying the amine which is coupled with optionally substituted 4-chloroquinazoline (such as 9-3 shown below).

### Preparation of A9

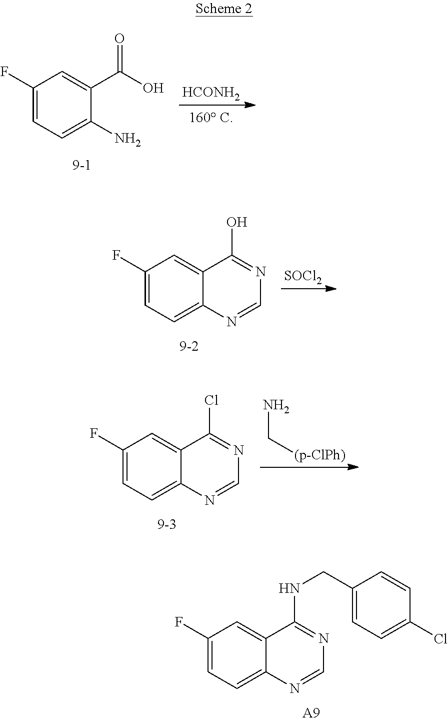

To a suspension of AgNO₂ (448.5 mg, 2.92 mmol) in diethyl ether (5 mL) was added compound 9-1 (500 mg, 2.65 mmol) dropwise in an ice-salt bath under Argon. The mixture was warmed to RT and stirred overnight. The reaction mixture was filtered and the filtrate was concentrated in vacuo. The residue was purified by silica gel chromatography (EA:PE, 1:100) to give three compounds. By ¹H NMR it was difficult to judge which was the desired compound 9-2.

The mixture containing compound 9-2 (150 mg, 0.967 mmol, MC0449-41-2) and KOH (81.4 mg, 1.451 mmol) in CH₃CN/H₂O (1 mL/1 mL) was stirred for 2 h at RT. Then selectfluor (514.0 mg, 1.451 mmol) was added in one portion. The mixture was stirred overnight at RT. The reaction mixture was poured into water (10 mL), extracted with ethyl acetate (2×20 mL). The combined organics were washed with brine (10 mL), dried over MgSO4, concentrated and purified by silica gel chromatography (PE) to afford compound 9-3 as a colorless oil (70 mg, yield: 42%).

To a solution of compound 9-3 (50 mg, 0.27 mmol) and (4-chlorophenyl)methanamine (47 mg, 0.33 mmol) in isopropyl alcohol (5 mL) was added Et₃N (46 μL, 0.33 mmol). The solution was microwaved for 20 min at 150° C. TLC showed the reaction was completed. The mixture was concentrated and purified by flash chromatography to give A9 as a light yellow solid (52 mg, yield: 67%, confirmed by ¹H NMR, and LC-MS). The ¹H NMR is shown in FIG. 23.

### Preparation of A30

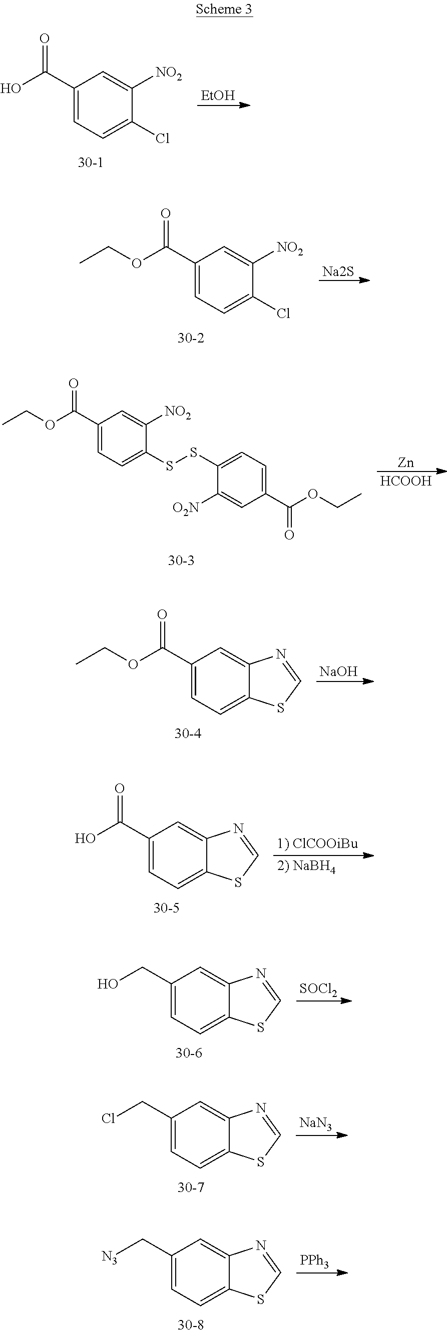

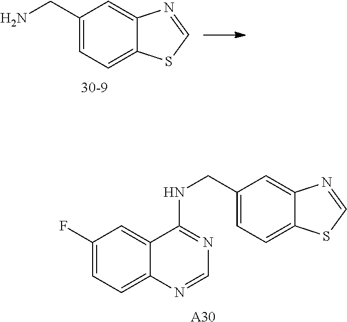

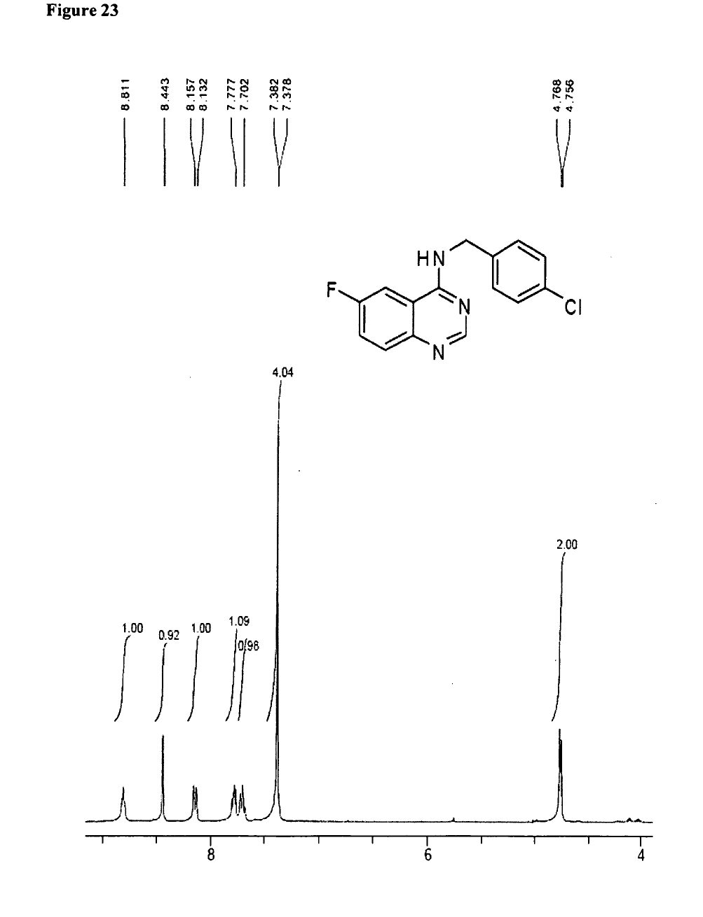

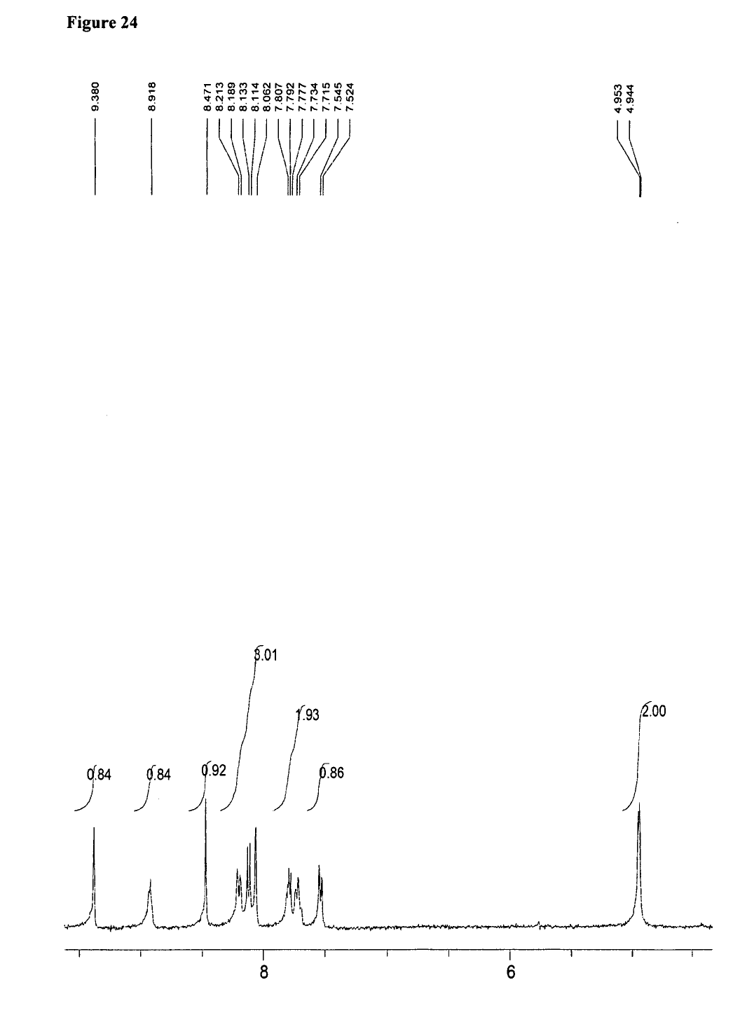

A solution of compound 9-3 (105 mg, 0.573 mmol), 30-9 (94 mg, 0.573 mmol) and NEt₃ (0.22 mL, 1.64 mmol) in isopropanol (4 mL) was microwaved at 150° C. for 20 min. Concentration and purification by column chromatography gave A30 as a yellow solid (80 mg, yield: 45%, confirmed by ¹H NMR). The ¹H NMR is shown in FIG. 24.

### Preparation of A36

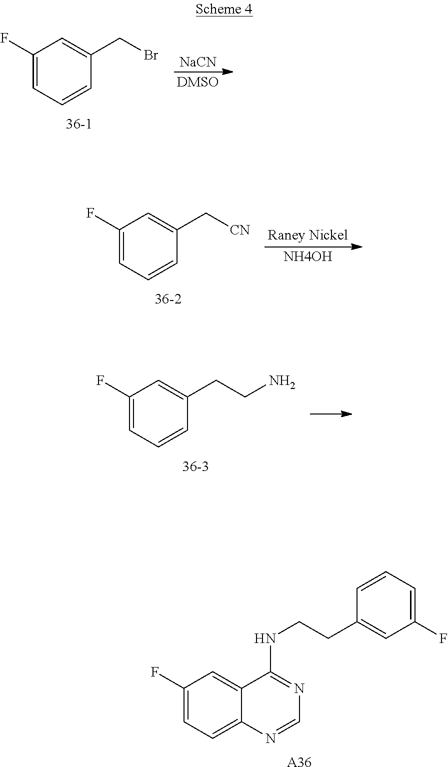

A solution of compound 36-1 (1.0 g, 5.3 mmol) and NaCN (520 mg, 10.6 mmol) in DMSO (10 mL) was stirred at 30° C. overnight. TLC showed the reaction was completed. The mixture was diluted with water (30 mL), and extracted with ethyl acetate (50 mL). The organic layer was washed by water (10 mL×5) and NaHCO₃ (sat., 20 mL), dried over anhydrous Na₂SO₄, and concentrated. The residue was purified by flash chromatography to give 36-2 as colorless oil (360 mg, yield: 50%).

To a solution of compound 36-2 (346 mg, 2.56 mmol) in THF (10 mL) was added Raney Ni. Then the mixture was adjusted to PH=10 with concentrated aqueous ammonia and stirred at 30° C. overnight. TLC showed the reaction was completed. The mixture was filtered through Celite and the filtrate was concentrated to give 36-3 as a yellow oil (120 mg, yield: 34%).

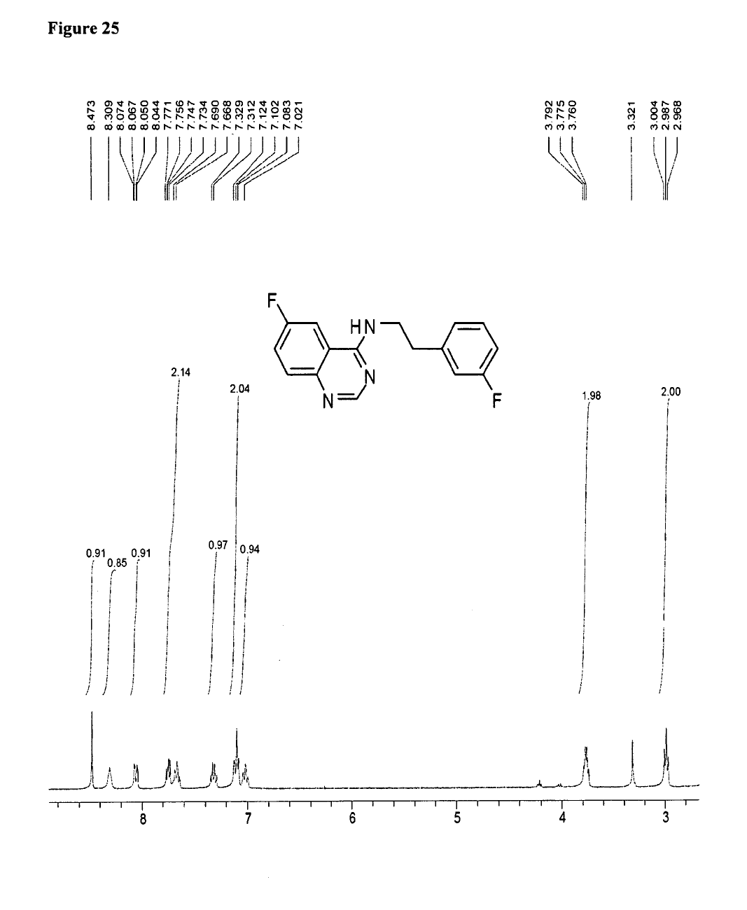

To a solution of compound 9-3 (50 mg, 0.27 mmol) and 36-3 (46 mg, 0.33 mmol) in isopropyl alcohol (5 mL) was added Et₃N (46 uL, 0.33 mmol). The solution was microwaved for 20 min at 150° C. TLC showed the reaction was completed. Concentration and purification by flash chromatography gave A36 as a white solid (48.4 mg, yield: 63%, confirmed by ¹H NMR at 400 MHz in DMSO, and MS). The ¹H NMR is shown in FIG. 25.

### Example 9

### Separating Autophagy-Inhibiting Activity from PDE5-Inhibiting Activity

The structural activity relationship (SAR) of MBCQ derivatives was investigated to determine if its activity in inhibiting autophagy may be separated from its PDE5 inhibitory activity. Among the MBCQ derivatives synthesized and analyzed for their autophagy inhibiting activity, as described above, some compounds exhibited autophagy inhibitory activity similar or above that of MBCQ and others had no anti-autophagy activity and thus can serve as negative controls.

Fourteen MBCQ derivatives were selected and screened for their activities on PDE5 (Wang, H., Yan, Z., Yang, S., Cai, J., Robinson, H., and Ke, H. (2008). Kinetic and structural studies of phosphodiesterase-8A and implication on the inhibitor selectivity. Biochemistry 47, 12760-12768). Among them, C43 (6-fluoro-N-(4-fluorobenzyl)quinazolin-4-amine), an effective autophagy inhibitor with IC₅₀ of 0.87 μM which is comparable to that of MBCQ, was found to have much reduced inhibiting activity towards PDE5 and other PDEs. Thus, the PDE5 inhibiting activity of MBCQ can be chemically separated from that of autophagy inhibiting activity.

TABLE 1
Summary of Determination of % Inhibition of PDE5 activity

| | Concentration = | | Concentration = | | Concentration = | |
|---|---|---|---|---|---|---|
| Target | 20 μM | | 2 μM | | 0.2 μM | |
| I.D. | Average | SD | Average | SD | Average | SD |
| A35 | 36.0 | 10.7 | −17.0 | 4.6 | −12.4 | 1.0 |
| A37 | −2.0 | 8.4 | −13.7 | 10.8 | −19.5 | 11.6 |
| A41 | −9.1 | 4.2 | −18.1 | 6.5 | −8.0 | 5.2 |
| A64 | 7.59 | 7.14 | 1.97 | 1.12 | −3.35 | 8.68 |
| A68 | 0.92 | 7.99 | −2.71 | 9.86 | −14.46 | 3.61 |
| A69 | −1.96 | 3.92 | −2.84 | 6.29 | 1.78 | 11.76 |
| A70 | −8.55 | 6.00 | −2.88 | 2.79 | −11.26 | 7.73 |
| A72 | −2.07 | 7.16 | −0.23 | 0.77 | −0.15 | 9.36 |
| Sildenafil | 101.7 | 2.0 | 98.8 | 2.0 | 96.5 | 3.9 |
| Zaprinast | 101.6 | 3.0 | 88.4 | 2.6 | 38.0 | 3.5 |
| MBCQ | 98.8 | 4.7 | 91.0 | 10.4 | 54.4 | 11.1 |

Consistent with this conclusion, there were a number of other known PDE5 inhibitors in the bioactive library that were screened, including MY-5445, dipyridamole, IBMX and sildenafil, but not recovered as autophagy inhibitors. To further confirm this conclusion, H4-LC3-GFP cells were treated with rapamycin and other PDE5 inhibitors including MY-5445 (30 μM), dipyridamole (80 μM), IBMX (100 μM) or sildenafil (10 μM) using MBCQ as a positive control. None of the PDE5 inhibitors tested, including the most potent PDE5 inhibitor, sildenafil (Viagra) which has an EC₅₀ of 2.5 nM for PDE5, has any activity on autophagy. From these data, it was concluded that the autophagy inhibitory activity of MBCQ is not related to its PDE5 inhibitory activity.

### Example 10

### Identification of a Deubiquitinating Protease Complex for Vps34 Complex I

Ubiquitination represents an essential key step in mediating proteasomal degradation. Experiments were therefore run to determine if ubiquitination of Beclin1 is increased in cells treated with C43. As depicted in FIG. 16, it was found that C43 promoted the ubiquitination of Beclin1.

It was therefore hypothesized that C43 targets a deubiquitinating protease complex (DUB) which normally functions to negatively regulate the ubiquitination of Vps34 complex I. This follows the common finding that a small molecule is more likely to be an inhibitor than an activator. To directly test this hypothesis, a collection of 127 siRNAs targeting Human Deubiquitinating Enzymes from Dharmacon library SMART pools were screened for DUBs that when knockeddown lead to inhibition of autophagy using LC3-GFP-H4 cells as an assay.

siPLK1 was used for validation of transfection effiency, and siVps34 was included in as a positive control. Seventy-two hours post-transfection, cells were treated with DMSO, rapamycin (200 nM) to induce autophagy, or rapamycin (200 nM) and spautin (10 μM), respectively in duplicate for additional 8 h. Cells were counterstained with Hoechst 33342 (0.5 μM) and fixed in 3.8% PFA. The fluorescent images were acquired and quantified using a CellWoRx High Content Cell Analysis System.

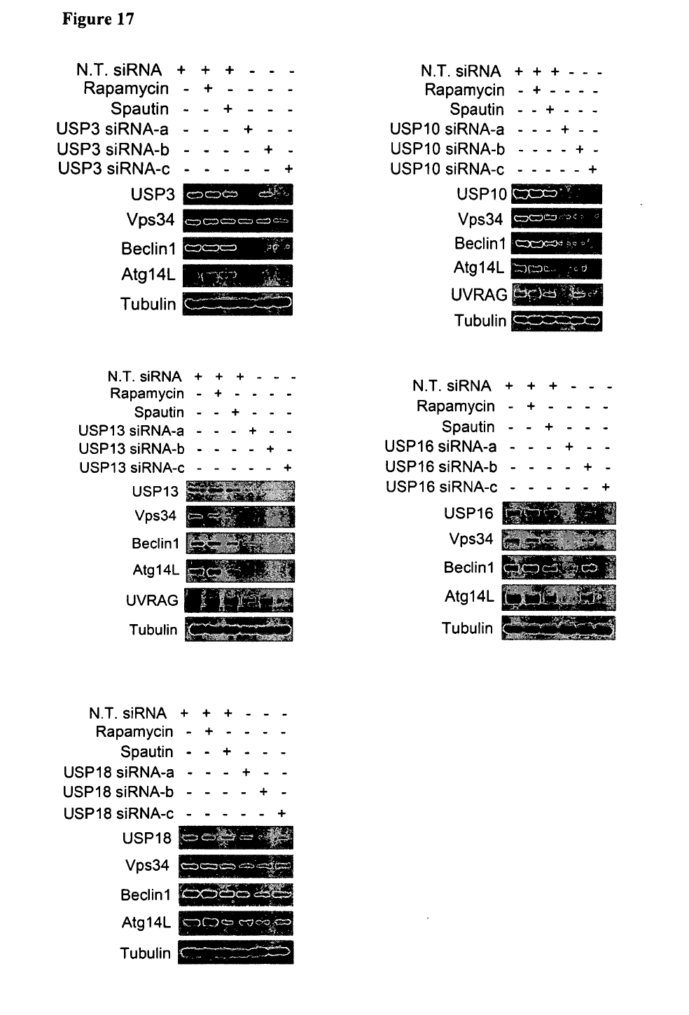

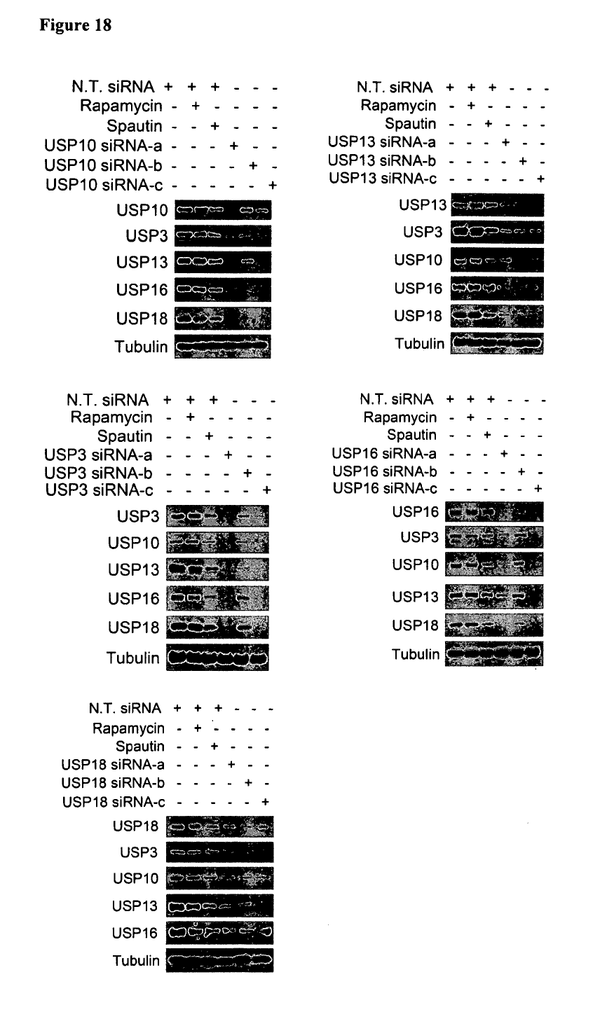

The screen identified USP10, USP13, USP3, USP16 and USP18 as five genes that when knockdown led to a decrease in the levels of autophagy under the basal condition as well as in the presence of rapamycin by at least 1.5 standard deviation from the plate median. The effects of knockdown of these five USPs on the protein expression levels in the Vps34 complexes in H4 cells were analyzed. It was found that knockdown of any of the five USPs reduced the levels of endogenous Vps34, Beclin1, Atg14L and UVRAG (FIG. 17). Furthermore, knockdown of any of the five USPs also led to reductions in the protein levels of the other four USPs (FIG. 18). Interestingly, the treatment of C43 also reduced the levels of these five USPs (FIG. 18). Treatment of spautin also can reduce the levels of USP13 and USP10 in 293T cells and Bcap-37 cells, but have little effect on the levels of USP44, an unrelated USP.

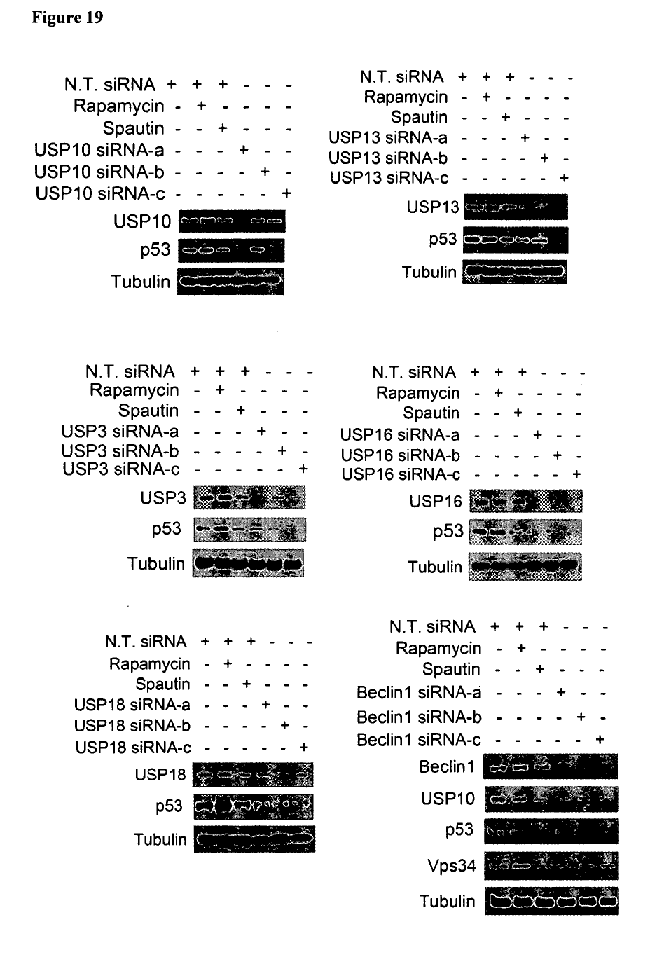

These results suggest that the stabilities of USP3, USP10, USP13, USP16 and USP 18 are co-dependent upon each other which might happen if they exist in a large complex. To test this posibility, GFP-USP10 and Myc-USP13 plasmids were transfected into 293T cells and examined by GFP-USP10 interaction and Myc-USP13 immunoprecipitation. It was found found that GFP-USP10 and Myc-USP13 could indeed interact and importantly, the interaction was inhibited in spautin-treated cells (FIG. 19). Thus, it was concluded that spautin disrupts the USP10 and USP13 interaction which might be needed for appropriately targeting this deubiquitinating protease complex to regulate the ubiquitination status of Vps34 complexes.

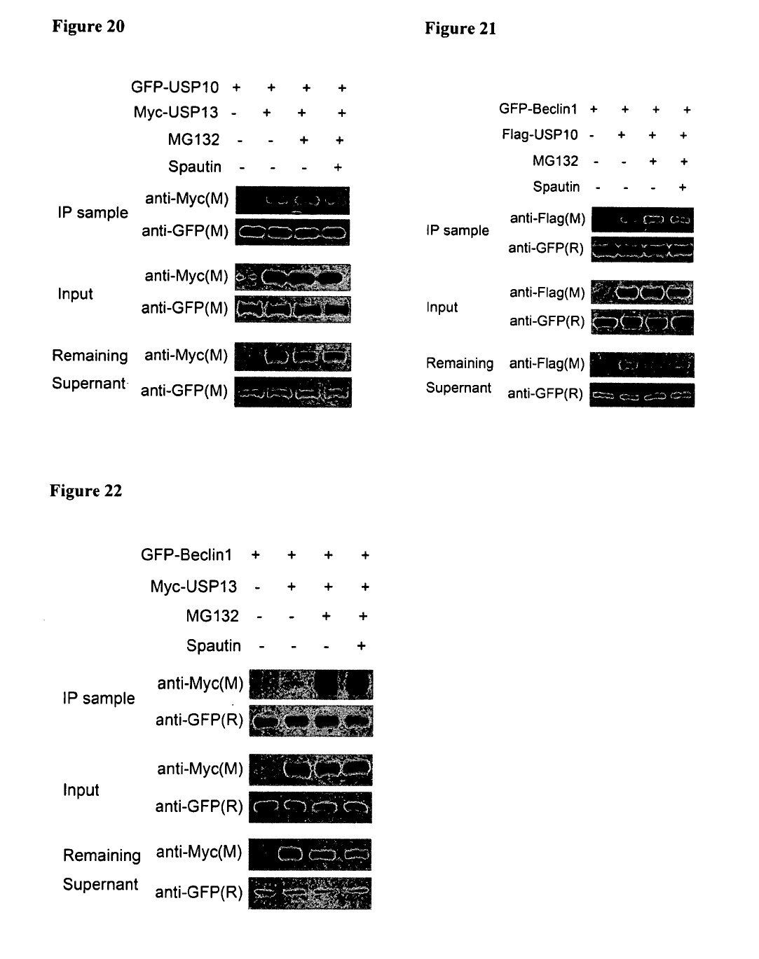

Since USP10 is known as the DUB of p53, the effects of knocking down these USPs on p53 was also investigated. It was found that the knockdown of anyone of the five USPs could lead to the reduction of p53 (FIG. 20). These data suggest that USP3, USP10, USP13, USP16 and USP18 are all regulators of p53.

To further confirm that USP10 and USP13 are the deubiquitinating proteases of Vps34 complexes, the interaction of Flag-USP10/GFP-Beclin1 and Myc-USP13/GFP-Beclin1 in 293T cells was assayed with immunoprecipitation. It was found that both Flag-USP10 and Myc-USP13 could interact with GFP-Beclin1; and interestingly, the treatment of spautin could impair the interaction of Flag-USP10 with GFP-Beclin1 (FIG. 21A), but not Myc-USP13 with GFP-Beclin1 (FIG. 21B). This result suggests that spautin may target on or upstream of USP10 to disrupt the interaction of USP10 and Beclin1. Importantly, it was also found that the knockdown of Beclin1 or Vps34 could reduce the endogenous levels of USP10 and p53, which is known as the substrate of USP10 (FIG. 21C). This suggests that Vps34 complexes may be able to regulate their own levels by stabilizing its deubiquitinating protease including USP10 and USP13. Furthermore, this may provide a mechanism to explain why beclin1 is frequently lost in many kinds of cancers as the loss of beclin1 may lead to a reduction of p53 by inhibiting its deubiquitining proteases.

### INCORPORATION BY REFERENCE

All of the U.S. patents and U.S. published patent applications cited herein are hereby incorporated by reference.

### Equivalents

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

1. A compound represented by formula I: 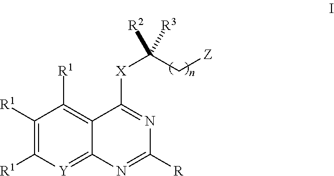
or a pharmaceutically acceptable salt, biologically active metabolite, solvate, hydrate, prodrug, enantiomer or stereoisomer thereof, wherein
n is 0, 1, 2, 3 or 4;
Y is —C(R¹)═ or —N═;
R is —H, lower alkyl, —CH₃, lower fluoroalkyl, —CH₂F, —CHF₂, —CF₃, —NO₂, —OH, —NH₂, —NH(lower alkyl), —N(lower alkyl)₂, or lower alkynyl;
R¹ is independently selected for each occurrence from the group consisting of —H, —F, —Cl, —Br, —I, —NO₂, —OH, —NH₂, —NH(lower alkyl), —N(lower alkyl)₂, —CH₃, —CF₃, —C(═O)(lower alkyl), —CN, —O(lower alkyl), —O(lower fluoroalkyl), —S(═O)(lower alkyl), —S(═O)₂(lower alkyl) and —C(═O)O(lower alkyl);
R² and R³ are independently selected from the group consisting of —H, lower alkyl, lower fluoroalkyl, lower alkynyl and hydroxyalkyl;
X is —O—, —S—, —N(H)—, —N(lower alkyl)-, —CH₂—, —CH₂CH₂—, —CH₂CH₂CH₂—, —CH₂CH₂CH₂CH₂—, —CH₂CH₂CH₂CH₂CH₂— or —CH₂CH₂CH₂CH₂CH₂CH₂—; and
Z is phenyl, pyridyl, vinyl, morphinyl, phenanthrolinyl, naphthyl, furyl or benzo[d]thiazolyl; and optionally substituted with one or more substitutents selected from the group consisting of —CH₃, lower alkyl, fluoroalkyl, —OCH₃, —OCF₃, lower fluoroalkoxy, —F, —Cl, —Br, —I, —NO₂, lower alkyoxy, —NH(lower alkyl), —N(lower alkyl)₂, —CF₃, and 3,4-methylene dioxy;
provided that the compound is not 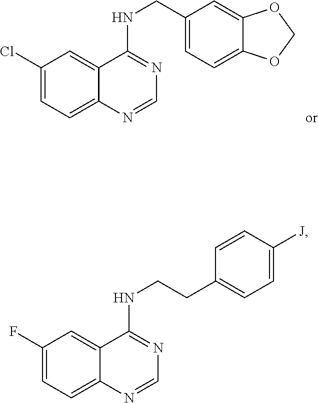
wherein J is Cl, OCHF₂, OCH₂CH₃, OCH₂CF₃, O(CH₂)₂CH₃, OCH(CH₃)₂, O(CH₂)₃CH₃, or O(cyclopentyl).

2. A compound of claim 1, wherein Y is —C(R¹)═.

3. A compound of claim 1, wherein n is Y is —N═.

4. A compound of claim 1, wherein R is —H.

5. A compound of claim 1, wherein at least one R¹ is —NH₂, —Cl, —NO₂, —I, or —OMe.

6. (canceled)

7. A compound of claim 1, wherein R² is —CH₃.

8. A compound of claim 1, wherein R² is —H.

9. A compound of claim 1, wherein R³ is —CH₃.

10. A compound of claim 1, wherein R³ is —H.

11. A compound of claim 1, wherein X is —O—, —S—, —N(H)—, —N(lower alkyl)- or —CH₂—.

12-13. (canceled)

14. A compound of claim 1, wherein Z is phenyl optionally substituted with one or more substitutents selected from the group consisting of —CH₃, lower alkyl, fluoroalkyl, —OCH₃, —OCF₃, lower fluoroalkoxy, —F, —Cl, —Br, —I, —NO₂, lower alkyoxy, —NH(lower alkyl), —N(lower alkyl)₂, —CF₃, and 3,4-methylene dioxy.

15-38. (canceled)

39. A compound, or a pharmaceutically acceptable salt thereof, selected from the group consisting of

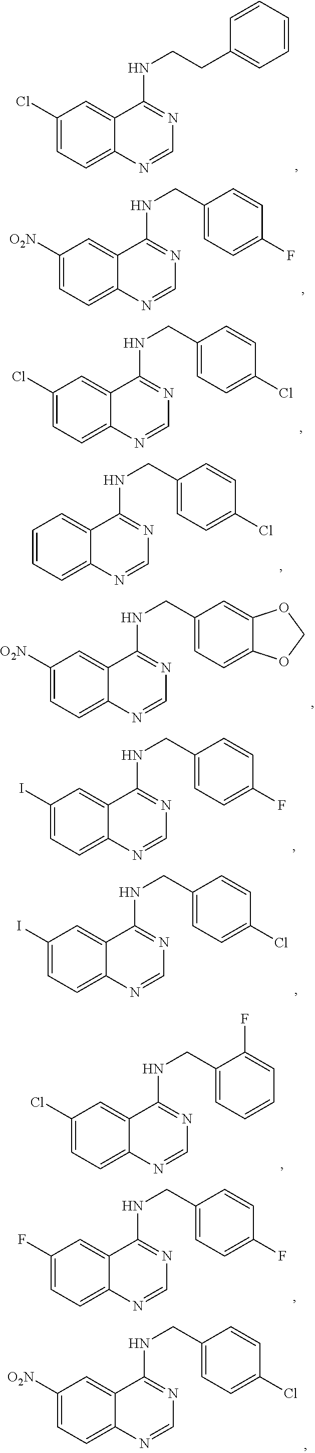

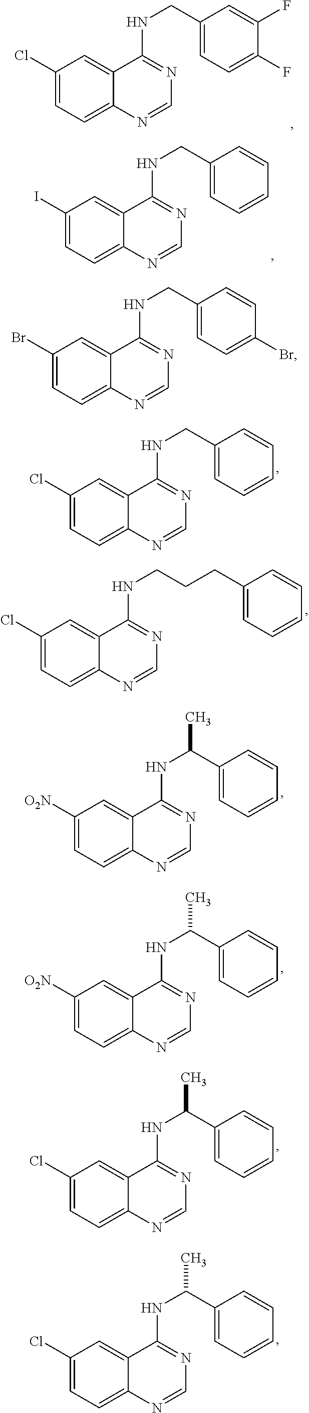

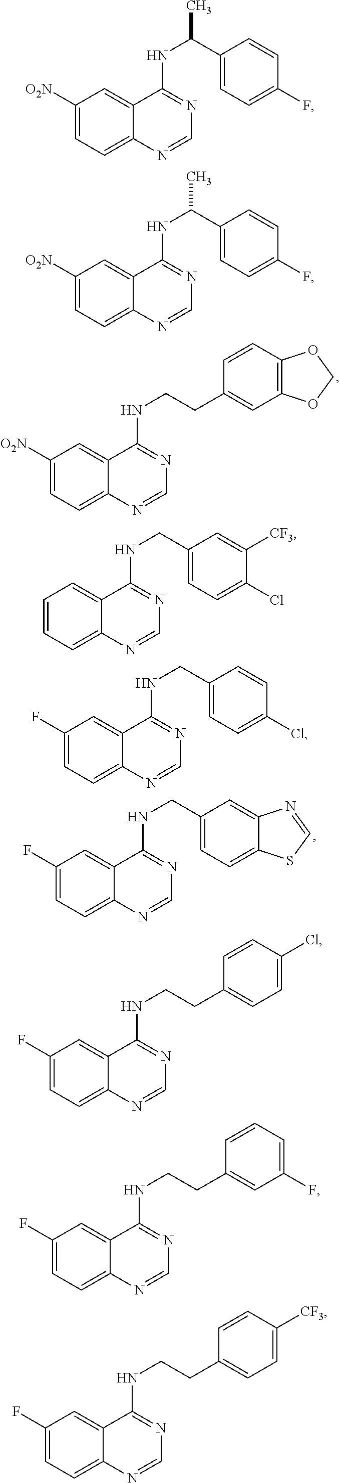

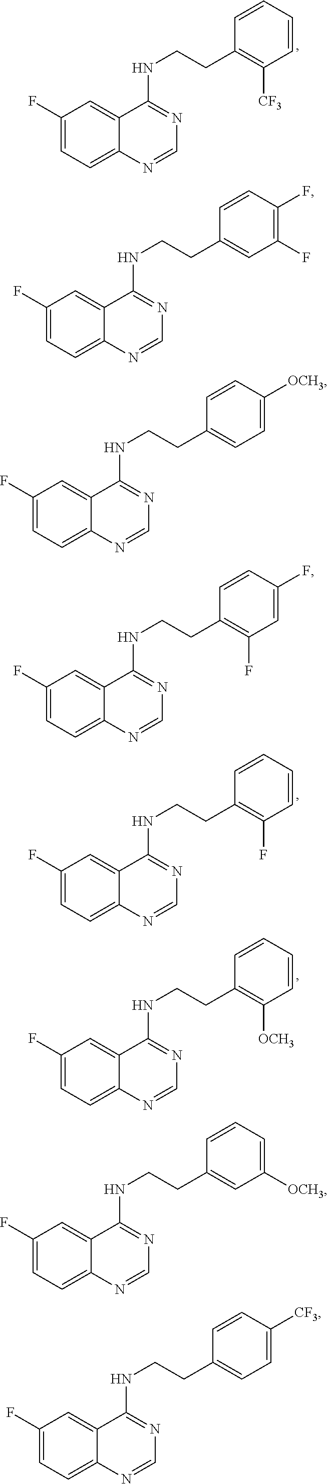

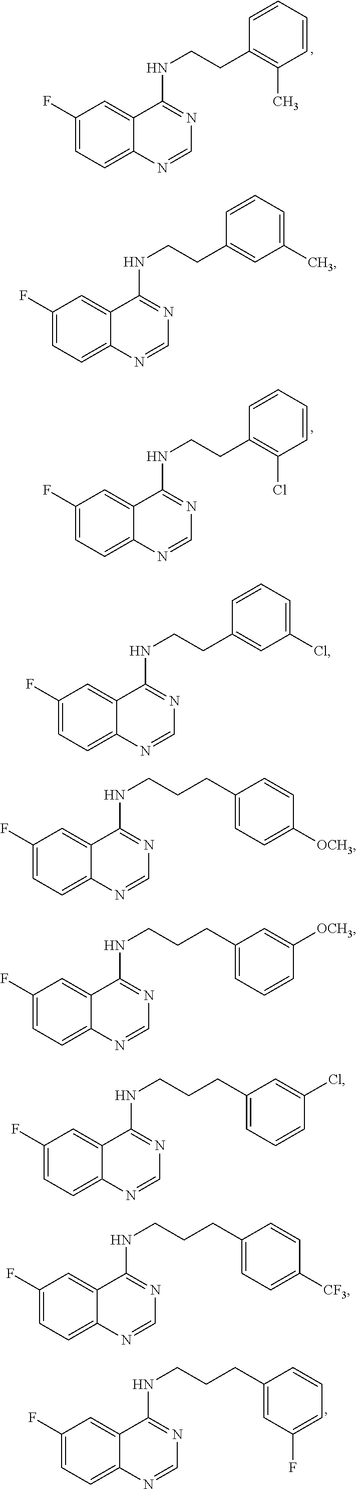

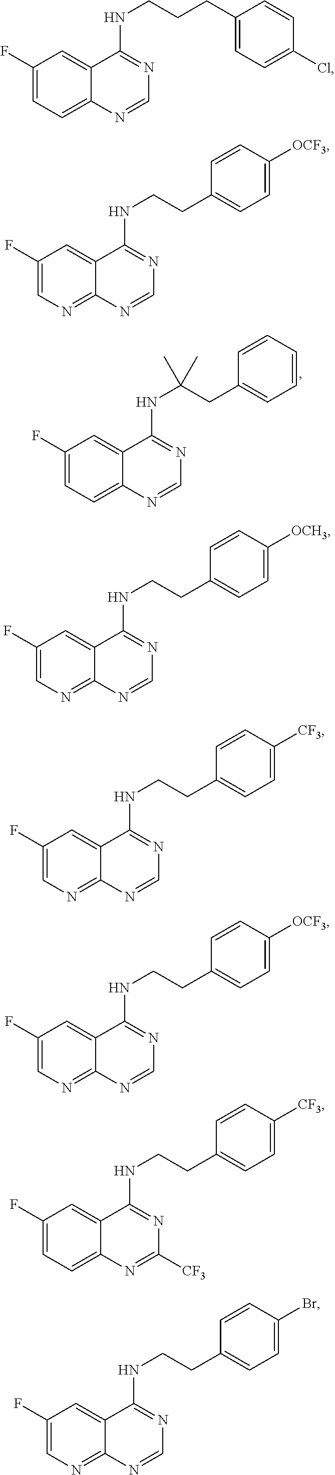

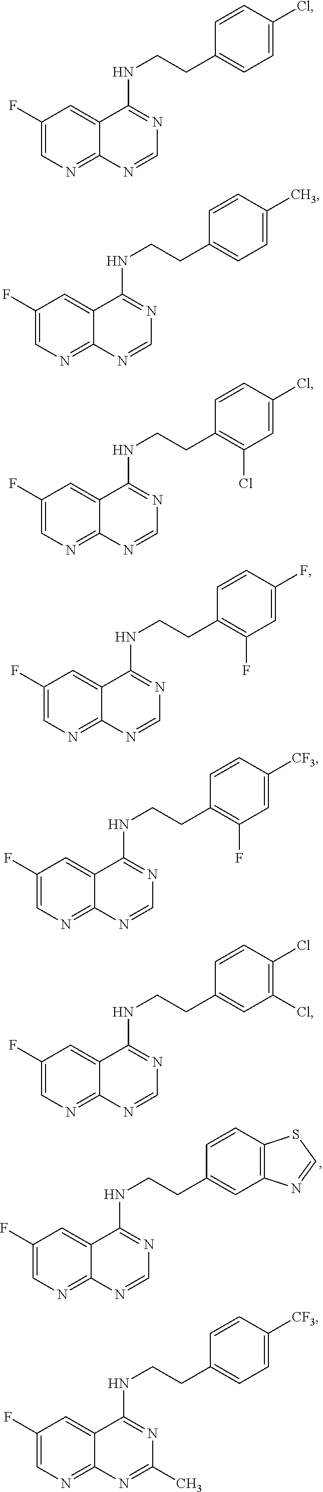

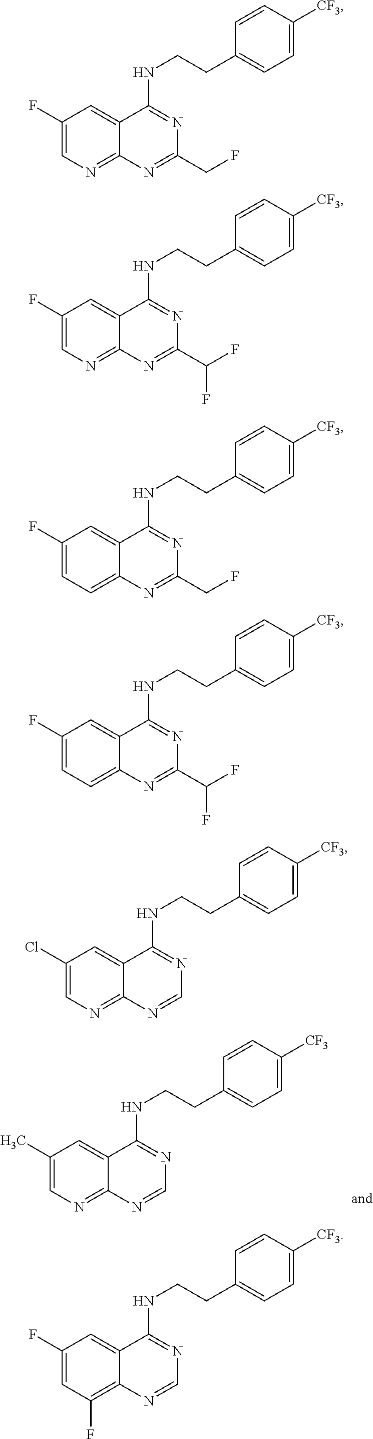

40. (canceled)

41. A method of treating cancer, pancreatitis, neurodegeneration, an inflammatory disease, an infectious disease, or an infection caused by an intracellular pathogen, comprising the step of administering to a subject in need thereof a therapeutically effective amount of one or more compounds of claim 1 or claim 39.

42. The method of claim 41, wherein the method is for treating cancer.

43. The method of claim 42, wherein said cancer is selected from the group consisting of leukemia, non-small cell lung cancer, colon cancer, central nervous system cancer, melanoma, ovarian cancer, renal cancer, prostate cancer, and breast cancer.

44. The method of claim 41, wherein the method is for treating pancreatitis.

45. The method of claim 41, wherein the method is for treating neurodegeneration.

46. The method of claim 41, wherein the method is for treating a neurodegenerative condition selected from the group consisting of vascular dementia, presenile dementia, neurodegeneration in Down syndrome, and HIV-related dementia.

47. The method of claim 41, wherein the method is for treating neurodegeneration; and the method enhances cognition or inhibits cognitive decline in said subject having said neurodegenerative condition.

48. The method of claim 41, wherein the method is for treating an infection caused by an intracellular pathogen.

49. The method of claim 48, wherein the infection is caused by a bacteria or virus.

50-56. (canceled)

57. A method of inactivating a deubiquitinating protease complex comprising the step of contacting the deubiquitinating protease complex with one or more compounds of claim 1 or claim 39; wherein the deubiquitinating protease complex comprises USP3,USP10, USP13, USP16 and USP18.
